SEQUENCE LISTING (89 sequences)

SEQ ID NO: 1 - protein, 13 aa, Artificial Sequence
  FXII618
RCFRLPCRQLRCR

SEQ ID NO: 2 - protein, 11 aa, Artificial Sequence
  Cons. Seq.
  VARIANT 1: Ala, Cys, Asp, Glu, Phe, Gly, His, Ile, Lys, Leu, Met, Asn, Pro, Gln, Arg, Ser, Thr, Val, Trp, Tyr
  VARIANT 11: Ala, Cys, Asp, Glu, Phe, Gly, His, Ile, Lys, Leu, Met, Asn, Pro, Gln, Arg, Ser, Thr, Val, Trp, Tyr
XCWGACLNVCX

SEQ ID NO: 3 - protein, 10 aa, Artificial Sequence
  Cons. Seq.
  VARIANT 1: Ala, Cys, Asp, Glu, Phe, Gly, His, Ile, Lys, Leu, Met, Asn, Pro, Gln, Arg, Ser, Thr, Val, Trp, Tyr
  VARIANT 7: Ala, Cys, Asp, Glu, Phe, Gly, His, Ile, Lys, Leu, Met, Asn, Pro, Gln, Arg, Ser, Thr, Val, Trp, Tyr
  VARIANT 8: la, Cys, Asp, Glu, Phe, Gly, His, Ile, Lys, Leu, Met, Asn, Pro, Gln, Arg, Ser, Thr, Val, Trp, Tyr
  VARIANT 10: Ala, Cys, Asp, Glu, Phe, Gly, His, Ile, Lys, Leu, Met, Asn, Pro, Gln, Arg, Ser, Thr, Val, Trp, Tyr
XCCYLRXXCX

SEQ ID NO: 4 - protein, 17 aa, Artificial Sequence
  Cons. Seq.
  VARIANT 1: Ala, Cys, Asp, Glu, Phe, Gly, His, Ile, Lys, Leu, Met, Asn, Pro, Gln, Arg, Ser, Thr, Val, Trp, Tyr
  VARIANT 7: Ala, Cys, Asp, Glu, Phe, Gly, His, Ile, Lys, Leu, Met, Asn, Pro, Gln, Arg, Ser, Thr, Val, Trp, Tyr
  VARIANT 8: Ala, Cys, Asp, Glu, Phe, Gly, His, Ile, Lys, Leu, Met, Asn, Pro, Gln, Arg, Ser, Thr, Val, Trp, Tyr
  VARIANT 10: Ala, Cys, Asp, Glu, Phe, Gly, His, Ile, Lys, Leu, Met, Asn, Pro, Gln, Arg, Ser, Thr, Val, Trp, Tyr
  VARIANT 11: Ala, Cys, Asp, Glu, Phe, Gly, His, Ile, Lys, Leu, Met, Asn, Pro, Gln, Arg, Ser, Thr, Val, Trp, Tyr
  VARIANT 12: Ala, Cys, Asp, Glu, Phe, Gly, His, Ile, Lys, Leu, Met, Asn, Pro, Gln, Arg, Ser, Thr, Val, Trp, Tyr
  VARIANT 13: Ala, Cys, Asp, Glu, Phe, Gly, His, Ile, Lys, Leu, Met, Asn, Pro, Gln, Arg, Ser, Thr, Val, Trp, Tyr
  VARIANT 14: Ala, Cys, Asp, Glu, Phe, Gly, His, Ile, Lys, Leu, Met, Asn, Pro, Gln, Arg, Ser, Thr, Val, Trp, Tyr
  VARIANT 15: Ala, Cys, Asp, Glu, Phe, Gly, His, Ile, Lys, Leu, Met, Asn, Pro, Gln, Arg, Ser, Thr, Val, Trp, Tyr
  VARIANT 17: Ala, Cys, Asp, Glu, Phe, Gly, His, Ile, Lys, Leu, Met, Asn, Pro, Gln, Arg, Ser, Thr, Val, Trp, Tyr
XCWGAAXXCXXXXXXCX

SEQ ID NO: 5 - protein, 11 aa, Artificial Sequence
  FXII501
KCWGACLNTCP

SEQ ID NO: 6 - protein, 11 aa, Artificial Sequence
  FXII502
RCWGACLNVCS

SEQ ID NO: 7 - protein, 11 aa, Artificial Sequence
  FXII503
KCWGACLNACP

SEQ ID NO: 8 - protein, 11 aa, Artificial Sequence
  FXII504
VCWGACLNVCP

SEQ ID NO: 9 - protein, 11 aa, Artificial Sequence
  FXII505
QCWGACLNVCN

SEQ ID NO: 10 - protein, 11 aa, Artificial Sequence
  FXII506
ACWGACQNVCR

SEQ ID NO: 11 - protein, 11 aa, Artificial Sequence
  FXII507
FCNPYCMMKCA

SEQ ID NO: 12 - protein, 11 aa, Artificial Sequence
  FXII508
DCNPYCQMKCM

SEQ ID NO: 13 - protein, 11 aa, Artificial Sequence
  FXII509
RCWYGCVPQCY

SEQ ID NO: 14 - protein, 11 aa, Artificial Sequence
  FXII510
FCTKECLQSCY

SEQ ID NO: 15 - protein, 11 aa, Artificial Sequence
  FXII511
RCLQKCFPPCL

SEQ ID NO: 16 - protein, 10 aa, Artificial Sequence
  FXII512
GCCYLRLSCR

SEQ ID NO: 17 - protein, 10 aa, Artificial Sequence
  FXII513
GCCYLRYLCS

SEQ ID NO: 18 - protein, 13 aa, Artificial Sequence
  FXII514
LCSRLPCEQLPCN

SEQ ID NO: 19 - protein, 13 aa, Artificial Sequence
  FXII515
LCPRLSCHQLGCS

SEQ ID NO: 20 - protein, 13 aa, Artificial Sequence
  FXII516
RCFRLPCAQLSCE

SEQ ID NO: 21 - protein, 13 aa, Artificial Sequence
  FXII517
NCQYMRCPPMSCL

SEQ ID NO: 22 - protein, 13 aa, Artificial Sequence
  FXII518
SCMATQCLNQLYG

SEQ ID NO: 23 - protein, 17 aa, Artificial Sequence
  FXII519
ACWGAAAGCVQGASNCG

SEQ ID NO: 24 - protein, 17 aa, Artificial Sequence
  FXII520
ACWGAAAGCVSVHQSCG

SEQ ID NO: 25 - protein, 17 aa, Artificial Sequence
  FXII521
ACWGAAAKCSQTWSGCG

SEQ ID NO: 26 - protein, 17 aa, Artificial Sequence
  FXII522
ACWGAAAKCPQTAAQCG

SEQ ID NO: 27 - protein, 17 aa, Artificial Sequence
  FXII523
ACWGAAATCNQQQLVCG

SEQ ID NO: 28 - protein, 17 aa, Artificial Sequence
  FXII524
ACWGAAAHCQLPKDRCG

SEQ ID NO: 29 - protein, 17 aa, Artificial Sequence
  FXII525
ACWGAAVGCNSPRGPCG

SEQ ID NO: 30 - protein, 17 aa, Artificial Sequence
  FXII526
ACWGAAVSCETQSSLCG

SEQ ID NO: 31 - protein, 17 aa, Artificial Sequence
  FXII527
ACWGAALSCNPQGSRCG

SEQ ID NO: 32 - protein, 17 aa, Artificial Sequence
  FXII528
ACWGAAQRCKVNQGECG

SEQ ID NO: 33 - protein, 17 aa, Artificial Sequence
  FXII529
ACWGAAQKCKMNGQNCG

SEQ ID NO: 34 - protein, 17 aa, Artificial Sequence
  FXII530
ACWGAAQLCGRPQMPCG

SEQ ID NO: 35 - protein, 17 aa, Artificial Sequence
  FXII531
ACWGAAQACIPQMAACG

SEQ ID NO: 36 - protein, 17 aa, Artificial Sequence
  FXII532
ACWGAAQHCQASNNSCG

SEQ ID NO: 37 - protein, 17 aa, Artificial Sequence
  FXII533
ACWGAASGCNQTGNDCG

SEQ ID NO: 38 - protein, 17 aa, Artificial Sequence
  FXII534
ACWGAASACQQGGAKCG

SEQ ID NO: 39 - protein, 17 aa, Artificial Sequence
  FXII535
ACWGAASRCTVISGRCG

SEQ ID NO: 40 - protein, 17 aa, Artificial Sequence
  FXII536
ACWGAARGCPDQHGVCG

SEQ ID NO: 41 - protein, 17 aa, Artificial Sequence
  FXII537
ACWGAAKLCGAARDGCG

SEQ ID NO: 42 - protein, 17 aa, Artificial Sequence
  FXII538
ACWGAAERCKQADNSCG

SEQ ID NO: 43 - protein, 13 aa, Artificial Sequence
  Cons. Seq.
  VARIANT 1: Ala, Cys, Asp, Glu, Phe, Gly, His, Ile, Lys, Leu, Met, Asn, Pro, Gln, Arg, Ser, Thr, Val, Trp, Tyr
  VARIANT 3: Ala, Cys, Asp, Glu, Phe, Gly, His, Ile, Lys, Leu, Met, Asn, Pro, Gln, Arg, Ser, Thr, Val, Trp, Tyr
  VARIANT 6: Ala, Cys, Asp, Glu, Phe, Gly, His, Ile, Lys, Leu, Met, Asn, Pro, Gln, Arg, Ser, Thr, Val, Trp, Tyr
  VARIANT 8: Ala, Cys, Asp, Glu, Phe, Gly, His, Ile, Lys, Leu, Met, Asn, Pro, Gln, Arg, Ser, Thr, Val, Trp, Tyr
  VARIANT 11: Ala, Cys, Asp, Glu, Phe, Gly, His, Ile, Lys, Leu, Met, Asn, Pro, Gln, Arg, Ser, Thr, Val, Trp, Tyr
  VARIANT 13: Ala, Cys, Asp, Glu, Phe, Gly, His, Ile, Lys, Leu, Met, Asn, Pro, Gln, Arg, Ser, Thr, Val, Trp, Tyr
XCXRLXCXQLXCX

SEQ ID NO: 44 - protein, 13 aa, Artificial Sequence
  FXII572
RCPRLPCAQLQCP

SEQ ID NO: 45 - protein, 13 aa, Artificial Sequence
  FXII573
ICPRLPCAQLRCV

SEQ ID NO: 46 - protein, 13 aa, Artificial Sequence
  FXII574
VCPRLPCGQLRCG

SEQ ID NO: 47 - protein, 13 aa, Artificial Sequence
  FXII575
ICPRLPCRQLSCM

SEQ ID NO: 48 - protein, 13 aa, Artificial Sequence
  FXII576
VCPRLPCHQLRCG

SEQ ID NO: 49 - protein, 13 aa, Artificial Sequence
  FXII577
RCPRLPCEQLRCA

SEQ ID NO: 50 - protein, 13 aa, Artificial Sequence
  FXII578
ICPRLPCAQLVCT

SEQ ID NO: 51 - protein, 13 aa, Artificial Sequence
  FXII579
LCPRLPCGQLRCT

SEQ ID NO: 52 - protein, 13 aa, Artificial Sequence
  FXII580
ICPRLPCGQLSCQ

SEQ ID NO: 53 - protein, 13 aa, Artificial Sequence
  FXII581
RCPRLPCEQLACG

SEQ ID NO: 54 - protein, 13 aa, Artificial Sequence
  FXII582
GCPRLPCAQLRCS

SEQ ID NO: 55 - protein, 13 aa, Artificial Sequence
  FXII583
ICPRLPCSQLRCA

SEQ ID NO: 56 - protein, 13 aa, Artificial Sequence
  FXII584
KCPRLPCGQLRCA

SEQ ID NO: 57 - protein, 13 aa, Artificial Sequence
  FXII585
ACPRLPCAQLPCR

SEQ ID NO: 58 - protein, 13 aa, Artificial Sequence
  FXII586
RCPRLPCSQLVCG

SEQ ID NO: 59 - protein, 13 aa, Artificial Sequence
  FXII587
RCPRLPCAQLECQ

SEQ ID NO: 60 - protein, 13 aa, Artificial Sequence
  FXII588
ICPRLPCAQLKCR

SEQ ID NO: 61 - protein, 13 aa, Artificial Sequence
  FXII589
LCPRLPCAQLRCP

SEQ ID NO: 62 - protein, 13 aa, Artificial Sequence
  FXII590
ACPRLPCSQLKCG

SEQ ID NO: 63 - protein, 13 aa, Artificial Sequence
  FXII591
FCPRLPCAQLKCS

SEQ ID NO: 64 - protein, 13 aa, Artificial Sequence
  FXII592
VCPRLACGQLRCD

SEQ ID NO: 65 - protein, 13 aa, Artificial Sequence
  FXII593
RCPRLMCAQLHCP

SEQ ID NO: 66 - protein, 13 aa, Artificial Sequence
  FXII594
GCPRLMCAQLQCW

SEQ ID NO: 67 - protein, 13 aa, Artificial Sequence
  FXII595
RCPRLACEQLHCV

SEQ ID NO: 68 - protein, 13 aa, Artificial Sequence
  FXII596
RCPRLPCSQLVCG

SEQ ID NO: 69 - protein, 13 aa, Artificial Sequence
  FXII597
VCPRLQCHQLRCT

SEQ ID NO: 70 - protein, 13 aa, Artificial Sequence
  FXII598
ICPRLACGQLRCE

SEQ ID NO: 71 - protein, 13 aa, Artificial Sequence
  FXII599
VCARLPCAQLKCQ

SEQ ID NO: 72 - protein, 13 aa, Artificial Sequence
  FXII600
ACFRLPCLQLACH

SEQ ID NO: 73 - protein, 13 aa, Artificial Sequence
  FXII601
RCFRLPCSQLTCV

SEQ ID NO: 74 - protein, 13 aa, Artificial Sequence
  FXII602
RCARLPCHQLHCL

SEQ ID NO: 75 - protein, 13 aa, Artificial Sequence
  FXII603
RCARLPCHQLKCS

SEQ ID NO: 76 - protein, 13 aa, Artificial Sequence
  FXII604
KCLRLFCDQLPCR

SEQ ID NO: 77 - protein, 13 aa, Artificial Sequence
  FXII605
GCFRLLCSQLKCA

SEQ ID NO: 78 - protein, 13 aa, Artificial Sequence
  FXII606
WCLRLQCEQLMCG

SEQ ID NO: 79 - protein, 13 aa, Artificial Sequence
  FXII607
RCPRLPCAQLSCE

SEQ ID NO: 80 - protein, 13 aa, Artificial Sequence
  FXII608
RCFRLPCAQLRCE

SEQ ID NO: 81 - protein, 13 aa, Artificial Sequence
  FXII609
RCPRLPCAQLRCE

SEQ ID NO: 82 - protein, 13 aa, Artificial Sequence
  FXII610
ACFRLPCAQLSCE

SEQ ID NO: 83 - protein, 13 aa, Artificial Sequence
  FXII611
RCFRLPCAQLSCA

SEQ ID NO: 84 - protein, 13 aa, Artificial Sequence
  FXII612
RCFRLPCEQLSCE

SEQ ID NO: 85 - protein, 13 aa, Artificial Sequence
  FXII613
RCFRLPCSQLSCE

SEQ ID NO: 86 - protein, 13 aa, Artificial Sequence
  FXII614
RCFRLPCRQLSCE

SEQ ID NO: 87 - protein, 13 aa, Artificial Sequence
  FXII615
RCFRLPCWQLSCE

SEQ ID NO: 88 - protein, 13 aa, Artificial Sequence
  FXII616
RCFRLPCRQLRCA

SEQ ID NO: 89 - protein, 13 aa, Artificial Sequence
  FXII617
RCFRLPCAQLRCR

(19) United States
(12) Patent Application Publication
(10) Pub. No.: US 2018/0118786 A1
(43) Pub. Date: May 3, 2018

(54) NOVEL lNHIBITORS OF THE ENZYME ACTIVATED FACTOR XII (FXIIA)

(71) Applicant: ECOLE POLYTECHNIQUE FEDERALE DE LAUSANNE (EPFL), Lausanne (CH)

(72) Inventors: Christian HEINIS, Bern (CH); Vanessa BAERISWYL, Zürich (CH); Simon MIDDENDORP, Bern (CH); Jonas Alfred Karl WILBS, Lausanne (CH)

(73) Assignee: ECOLE POLYTECHNIQUE FEDERALE DE LAUSANNE (EPFL), Lausanne (CH)

(21) Appl. No.: 15/569,893

(22) PCT Filed: Apr. 27, 2016
(86) PCT No.: PCT/EP2016/059435

§ 371 Date: Oct. 27, 2017

(30) Foreign Application Priority Data
Apr. 28, 2015 (EP) 15165508.1

(51) Int. Cl. C07K 7/52; C12N 9/64; C12Q 1/56; G01N 33/86

(52) U.S. Cl. CPC C07K 7/52; C12N 9/6451; A61K 38/00; G01N 33/86; C12Q 1/56

(57) ABSTRACT

The present invention relates to a bicyclic inhibitor of the coagulation enzyme activated factor XII (FXIIa) comprising or consisting of the peptide) (X¹)(X²)(X³)ₙ(X⁴)RL(X⁵)(X⁶)ₘ(X⁷)(X⁹)ₗ(X¹⁰)(X¹¹)(X¹²)(X¹³)(X¹⁴)ₖ(X¹⁵)(X¹⁶), wherein (X¹) is present or absent and, if present, is an amino acid; (X²) is an amino acid with a side chain; (X³) is an amino acid and n is between 0 and 3, preferably 0 or 1 and most preferably 0; (X⁴) is an aliphatic L-amino acid or a cyclic L-amino acid, preferably L, P or an aromatic L-amino acid, and most preferably an aromatic L-amino acid; (X⁵) is an amino acid; (X⁶) is an amino acid and m is between 0 and 3, preferably 0 or 1 and most preferably 0; (X⁷) is an amino acid with a side chain; (X⁹) is an amino acid and l is between 0 and 3, preferably 0 or 1 and most preferably 0; (X¹⁰) is an amino acid; (X¹¹) is an amino acid, preferably Q; (X¹²) is a hydrophobic L-amino acid, preferably an aliphatic L-amino acid, and is most preferably L; (X¹³) is an amino acid; (X¹⁴) is an amino acid and k is between 0 and 3, preferably 0 or 1 and most preferably 0, (X¹⁵) is an amino acid with a side chain; and (X¹⁶) is present or absent and, if present, is an amino acid; and wherein the side chains of (X²), (X⁷) and (X¹⁵) are connected via a connecting molecule, said connecting molecule having at least three functional groups, each functional group forming a covalent bond with one of the side chains of (X²), (X⁷) and (X¹⁵).

The present invention relates to a bicyclic inhibitor of the coagulation enzyme activated factor XII (FXIIa) comprising or consisting of the peptide (X¹)(X²)(X³)ₙ(X⁴)RL(X⁵)(X⁶)ₘ(X⁷)(X⁹)ₗ(X¹⁰)(X¹¹)(X¹²)(X¹³)(X¹⁴)ₖ(X¹⁵)(X¹⁶), wherein (X¹) is present or absent and, if present, is an amino acid; (X²) is an amino acid with a side chain; (X³) is an amino acid and n is between 0 and 3, preferably 0 or 1 and most preferably 0; (X¹) is an aliphatic L-amino acid or a cyclic L-amino acid, preferably L, P or an aromatic L-amino acid, and most preferably an aromatic L-amino acid; (X⁵) is an amino acid; (X⁶) is an amino acid and m is between 0 and 3, preferably C or 1 and most preferably 0; (X⁷) is an amino acid with a side chain; (X⁹) is an amino acid and l is between 0 and 3, preferably 0 or 1 and most preferably 0; (X¹⁰) is an amino acid; (X¹¹) is an amino acid, preferably 0; (X¹²) is a hydrophobic L-amino acid, preferably an aliphatic L-amino acid, and is most preferably L; (X¹³) is an amino acid; (X¹⁴) is an amino acid and k is between 0 and 3, preferably 0 or 1 and roost preferably 0, (X¹⁵) is an amino acid with a side chain; and (X¹⁶) is present or absent and, if present, is an amino acid; and wherein the side chains of (X²), (X⁷) and (X¹⁵) are connected via a connecting molecule, said connecting molecule having at least three functional groups, each functional group forming a covalent bond with one of the side chains of (X²), (X⁷) and (X¹⁶).

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Blood coagulation tests are routinely performed in clinical laboratories to assess the efficiency of coagulation in patients. In most coagulation tests, blood clotting is induced with a strong trigger and the time to coagulation is used as an indication of the coagulation potential. While these tests are useful diagnostically, they are limited in their ability to correlate with the bleeding or thrombotic risk of the patient. Newer, global coagulation assays such as thromboelastography (TEG), thrombeelastometry (TEM) and calibrated automated thrombography (CAT) deliver more parameters of the coagulation process and promise to better predict the coagulation potential. Major efforts have been made towards the use of TEG and TEM, viscoelastometric methods to test hemostasis in whole blood. They allow the study of the phases of clotting and subsequent lysis under mimicked vein flow. Viscoelastometric methods are already used in the clinics for perioperative bleeding management. TEG is employed as an analytical method in hospitals. In some TEG and CAT assays, the coagulation trigger tissue factor (TF) has to be used at low concentration in order to optimally reflect the coagulation potential in patients. In such assays, however, contact of FXII with sample tubes can initiate the intrinsic coagulation pathway and falsify the result. Corn trypsin inhibitor (CTI), a 13.6 kDa protein isolated from corn seeds inhibiting FXIIa with a Ki of 24 nM was so far used to suppress these effects.^{1, 2} It is thus a natural inhibitor of FXIIa. However, CTI does not fully block contact activation at concentrations typically used (30-100 μg/mL). In addition, recent studies showed that CTI inhibits also other relevant proteases such as FXIa.³ Another limitation of CTI is the high price: 50 μg research grade CTI normally used per assay cost 5-10 dollars, depending on the provider. TEM is routinely used in the clinics for perioperative bleeding management. Recently, a consortium for the use of TEM to discriminate between severe and mild hemophilic disorders has been created. Such a classification requires preforming an EXTEM test (TEM with specific induction of coagulation via the extrinsic pathway) triggered by low-TF concentrations. Both, TEM and low-TF induced TGA require the use of a contact activation inhibitor.

Several recent studies indicated that intrinsic coagulation is implicated in pathological coagulation and have featured the blood coagulation factor FXII as a therapeutic target.⁴⁻¹⁰ Physiologic molecules such as DNA, RNA, protein aggregates, polyphosphates, and collagen were reported to activate FXII and to trigger thrombosis.¹¹ FXII-deficient mice were found to be protected from thrombus formation while presenting a normal hemostasis.⁸ Data from cohort studies suggested that elevated plasma FXII levels are associated with an increased risk of coronary events.^{4, 12} At the same time, deficiencies in the intrinsic pathway have no serious pathological consequences. FXII-deficient individuals present a normal hemostatic capacity.^{13, 14} Antibody-mediated inhibition of FXII confirmed this finding in primates.⁷ A range of FXII inhibitors of various origins and modes of action have been developed. The RNA-aptamer R4cXII-1.9 and the mouse mAb 15H8 inhibit autoactivation of FXII.⁷ The recombinant hematophagous insect protein domain infestin 4 (rHA-infestin-4; Ki=0.3 nM)^{15, 16} and the phage display-derived humanized antibody 3F7 are direct inhibitors of the activated form of FXII (Ki=13 nM).⁶ They all show selective inhibition of the intrinsic pathway of coagulation in in vitro coagulation assays. 15H8, rHA-infestin-4 and 3F7 showed efficient in vivo protection from thrombus formation in various thrombosis models and species, without associated bleedings:^{4, 6, 7, 9, 10} Several small molecule FXIIa inhibitors were developed but they inhibited structurally related trypsin-like serine proteases.¹⁷ Recently a synthetic FXIIa inhibitor with high selectivity over related proteases (>100 fold) was developed.¹⁸ However, its potency (Ki=1.2 μM) did not allow to further develop it toward the generation of a therapeutic compound. Despite the key role of FXII in the intrinsic coagulation cascade, no selective synthetic FXII inhibitors with a binding constant in the nanomolar range currently exists.

Hence, there is an ongoing need for further inhibitors of FXIIa which overcome at least one of the disadvantages associated with the currently available inhibitors of FXIIa. Such inhibitors are inter olio of interest for the diagnostic and therapeutic applications described of inhibitors of FXIIa herein below. In particular, an inhibitor providing selective inhibition of FXIIa in the nanomolar range is needed. This need is addressed by the present invention.

Accordingly, the present invention relates in a first aspect to a cyclic inhibitor of the coagulation enzyme activated factor XII (FXIIa) comprising or consisting of the peptide (X¹)(X²)(X³)ₙ(X⁴)RL(X⁵)(X⁶)ₘ(X⁷)(X⁸), wherein (X¹) is present or absent and, if present, is an amino acid; (X²) is an amino acid with a side chain; (X³) is an amino acid and n is between 0 and 3, preferably 0 or 1 and most preferably 0; (X⁴) is an aliphatic L-amino acid or a cyclic L-amino acid, preferably L, P or an aromatic L-amino acid, and most preferably an aromatic L-amino acid; (X⁵) is an amino acid; (X⁶) is an amino acid and m is between 0 and 3, preferably 0 or 1 and most preferably 0; (X⁷) is an amino acid with a side chain; and (X⁸) is present or absent and, if present, is an amino acid; and wherein the side chains of (X²) and (X⁷) are connected via a connecting molecule, said connecting molecule having at least two functional groups, each functional group forming a covalent bond with one of the side chains of (X²) and (X⁷).

The term “comprise/comprising” is generally used in the sense of include/including, that is to say permitting the presence of one or more features or components. The terms “comprise” and “comprising” also encompass the more restricted terms “consist of” and “consisting of”.

As used in the specification and claims, the singular form “a”, “an” and “the” include plural references unless the context clearly dictates otherwise.

As used herein, “activated factor XII” or “FXIIa” (EC 3.4.21.38) refers to a trypsin-like serine protease that initiates, through contact with negatively charged surfaces (referred to as contact activation), the intrinsic coagulation pathway. It activates a cascade of proteases including FXI, FIX, FX and thrombin. Thrombin activates fibrinogen which eventually forms blood clots. In more detail, thrombin catalyzes the conversion of fibrinogen to fibrin which polymerizes and forms together with platelets a blood clot. In contrast to the extrinsic pathway, that is essential for blood clot formation after injury, the intrinsic pathway is not required for homeostasis. FXI-deficient individuals present a normal hemostatic capacity⁹. Several recent studies suggested that intrinsic coagulation is implicated in pathological coagulation and thus harmful^{3-7, 10, 11}. FXII-deficient mice were found to be protected from thrombus formation while presenting a normal hemostasis³. Antibody mediated inhibition of FXII confirmed this finding in primates⁴. Drugs targeting this coagulation factor may thus lead to the development of antithrombotic agents which would prevent thrombotic events without compromising hemostasis. In accordance with the present invention, FXIIa is preferably human or mouse FXIIa and most preferably human α-FXIIa or β-FXIIa.

The term “inhibitor” as used herein designates a compound that reduces the effectiveness of a catalyst in a catalyzed reaction. In accordance with the invention the catalyst is the coagulation enzyme FXIIa. The main reactions being catalyzed by FXIIa are the selective cleavage of Arg−/−Ile bonds in factor XI to form factor XIa. The term “cyclic inhibitor” means that one or more series of atoms in the inhibitor is/are connected to form a ring or cycle.

The term “peptide” designates short chains of amino acids linked by peptide bonds. The shortest possible peptide consists of two amino acids joined by a single peptide bond. In accordance with the first aspect of the present invention the peptide comprises at least six amino acids linked by peptide bonds. Peptides are distinguished from proteins or polypeptides on the basis of size, and comprise in general less than 50 amino acids.

The term “amino acid” as used herein refers to an organic compound composed of amine (—NH₂ and carboxylic acid (—COOH) functional groups, generally along with a side-chain specific to each amino acid. The simplest amino acid glycin does not have a side chain (formula H₂NCH₂COOH). In amino acids that have a carbon chain attached to the α-carbon (such as lysine) the carbons are labeled in the order α, β, γ, δ, and so on. In some amino acids, the amine group may be attached, for instance, to the α-, β- or γ-carbon, and these are therefore referred to as α-, β- or γ-amino acids, respectively. All amino acids are in accordance with the present invention preferably α-amino acids (also designated 2-, or alpha-amino acids) which generally have the generic formula H₂NCHRCOOH, wherein R is an organic substituent being designated “side-chain”). In the simplest α-amino acid alanine (formula: H₂NCHCH₃COOH) the side is a methyl group. More preferably, the amino acids are in accordance with the present invention L-α-amino acids, noting that L-amino acids are L-stereoisomers (or “left-handed” isomers).

As mentioned, the side-chain of an amino acid is an organic substituent, which is in the case of α-amino acids linked to the α-carbon atom. Hence, a side chain is a branch from the parent structure of the amino acid. Amino acids are usually classified by the properties of their side-chain. For example, the side-chain can make an amino acid a weak acid (e.g. amino acids D and E) or a weak base (e.g. amino acids K and R), and a hydrophile if the side-chain is polar (e.g. amino acids L and I) or a hydrophobe if it is non-polar (e.g. amino acids S and C). An aliphatic amino acid has a side chain being an aliphatic group. Aliphatic groups render the amino acid nonpolar and hydrophobic. The aliphatic group is preferably an unsubstituted branched or linear alkyl. Non-limiting examples of aliphatic amino acids are A, V, L, and I. In a cyclic amino acid one or more series of atoms in the side chain is/are connected to form a ring. Non-limiting examples of cyclic amino acids are P, F, W, Y and H. It is to be understood that said ring has to be held distinct from the ring that is formed by the connecting molecule having at least two functional groups, each functional group forming a covalent bond with one of the side chains of (X²) and (X⁷). While the former ring of a cyclic amino acid is a part of the side chain of a single amino acid the latter ring is formed between the side chains of the two amino acids (X²) and (X⁷) via the connecting molecule. Also the ring being formed between (X⁷) and (X¹⁵) via the connecting molecule—which is present in accordance with a preferred embodiment discussed herein below—is formed between the side chains of the two amino acids. An aromatic amino acid is the preferred form of a cyclic amino acid. In an aromatic amino acid the ring is an aromatic ring. In terms of the electronic nature of the molecule, aromaticity describes the way a conjugated ring of unsaturated bonds, lone pairs of electrons, or empty molecular orbitals exhibits a stronger stabilization than would be expected by the stabilization of conjugation alone. Aromaticity can be considered a manifestation of cyclic delocalization and of resonance. Non-limiting examples of cyclic amino acids are F, W, Y and H. A hydrophobic amino acid has a non-polar side chain making the amino acid hydrophobic. Non-limiting examples of hydrophobic amino acids are M, F, W, G, A, V, L and I. A polar, uncharged amino acid has a non-polar side chain with no charged residues. Non-limiting examples of polar, uncharged amino acids are S, T, N, Q, C, U and Y. A polar, charged amino acid has a non-polar side chain with at least one charged residue. Non-limiting examples of polar, charged amino acids are D, E, H, K and R.

The term “connecting molecule” as used herein refers a molecule which is capable of connecting the side chains of at least two amino acids via covalent bonds whereby a ring or cycle is formed. A covalent bond is a chemical bond that involves the sharing of electron pairs between atoms. For this purpose the connecting molecule comprises at least two functional groups. A functional group designates a specific group of atoms or bonds within the connecting molecule that is responsible for connecting the side chain of an amino acid of said at least two amino acids to the connecting molecule. For this purpose the functional group in general undergoes a chemical reaction with at least one atom in the side chain of the amino acid. The chemical reaction results in a covalent bond between the functional group and the side chain. Non-limiting examples of functional groups are alkyl, alkenyl, alkynyl, phenyl, benzyl, halo (such as fluoro or chloro), hydroxyl, carbonyl, aldehyde, haloformyl, carbonate ester, carboxylate, carboxyl, ester, methoxy, hydroperoxy, peroxy, ether, hemiacetal, hemiketal, acetal, ketal, orthoester, heterocycle, orthocarbonate ester, carboxamide, amine (including primary, secondary and tertiary amine), imine, azide, azo compound, (iso)cyanate, nitrate, nitrite, sulfhydryl, sulfide, disulfide, sulfinyl, sulfonyl, sulfin, sulfo, (iso)thiocyanate, carbonothiol, carbonothioyl, phosphino (such as phosphate), borono, boronate, boroniate and borino. The at least two functional groups of the connecting molecule may be the same or 2.0 different and are preferably the same.

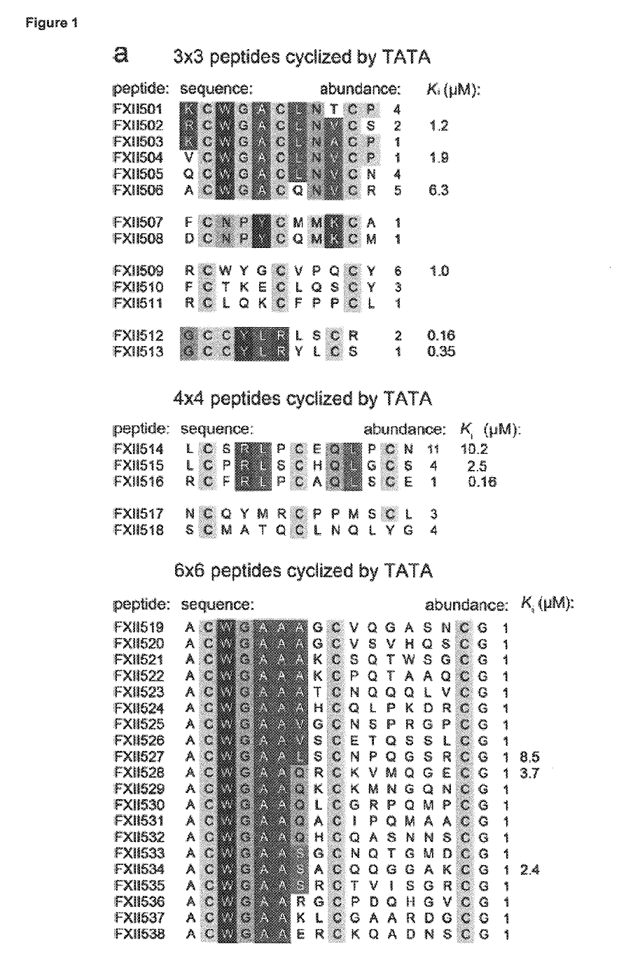

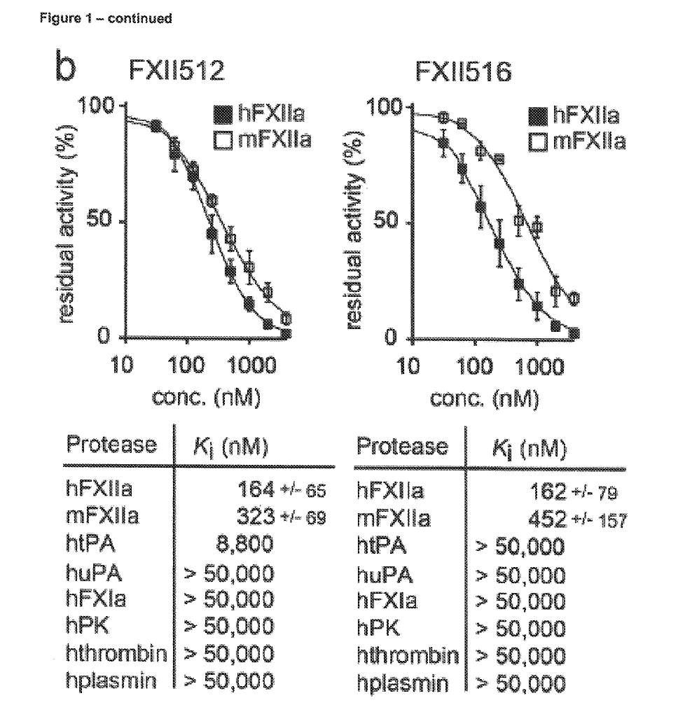

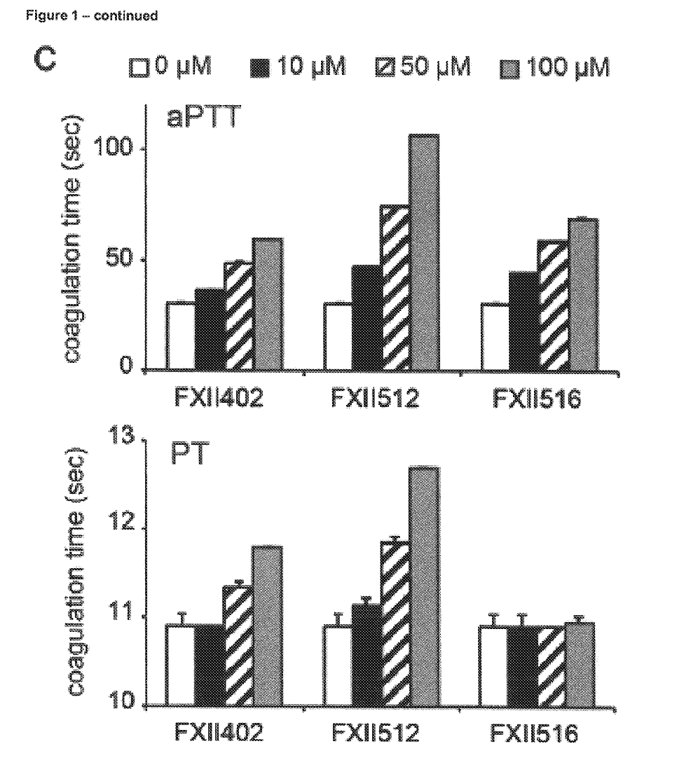

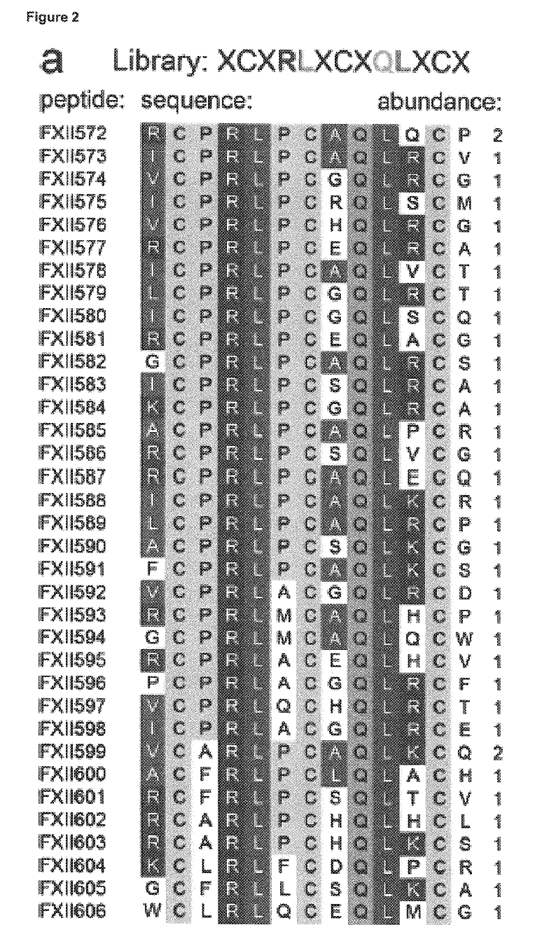

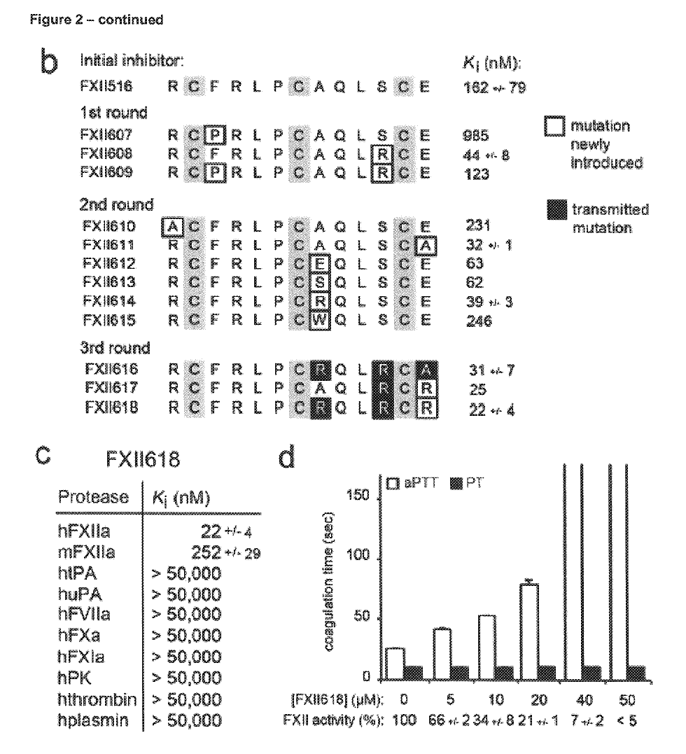

As can be taken from the examples herein below the peptide of SEQ ID NO: 1 (named FXII618 herein), has been obtained by screening large and structurally diverse bicyclic peptide phage display libraries. The excessive screening effort by phage display yielded a FXIIa inhibitor that is several orders of magnitude more potent and selective than existing small molecule FXIIa inhibitors. In more detail, from the libraries initially >1010 different bicyclic peptides against FXIIa were isolated. Further panning of the libraries (see FIG. 1) and affinity maturation efforts of the isolated bicyclic peptides against FXIIa (see FIG. 2) yielded a bicyclic peptide inhibitor of FXIIa with a Ki of 22 nM (FXII618), showing efficient and selective inhibition of FXIIa as evidenced by the inhibition of the intrinsic pathway of coagulation ex vivo and in vivo (see examples herein below). It is >200-fold more potent than the moderately selective small molecule FXIIa inhibitor 44, a coumarin derivative (Ki=4 μM) and around 8-fold more potent than the non-selective small molecule FXIIa inhibitor PCK (D-Pro-Phe-Arg chloromethyl ketone; IC50=180 nM). Furthermore, it is 60-fold more potent and >10-fold more selective than FXII402, being a bicyclic peptide FXIIa inhibitor recently presented and having a considerable distinct amino acid sequence as compared to FXII618¹⁵. FXII618 inhibits the initiation of the intrinsic pathway of coagulation in plasma and whole blood.

There is a considerable interest in the use of global coagulation assays in clinical laboratories. Suppression of the contact initiated coagulation pathway is essential when attempting to perform low tissue factor (TF)-dependent assays in whole blood or plasma samples. In diagnostic coagulation tests, such as thrombin generation assay (TGA) and thromboelastometry (TEM) described above, FXII618 can readily be used to prevent contact activated blood clotting during subsequent sample processing steps, thereby reducing in vitro artefacts. TGA is a coagulation test used in research labs and increasingly in the clinic. Major efforts have been made toward its clinical translation and thrombinoscope devices are expected to enter routine labs within the next few years (for example for patients with liver disease). Both, TEM and low-TF induced TGA require the use of a contact activation inhibitor.

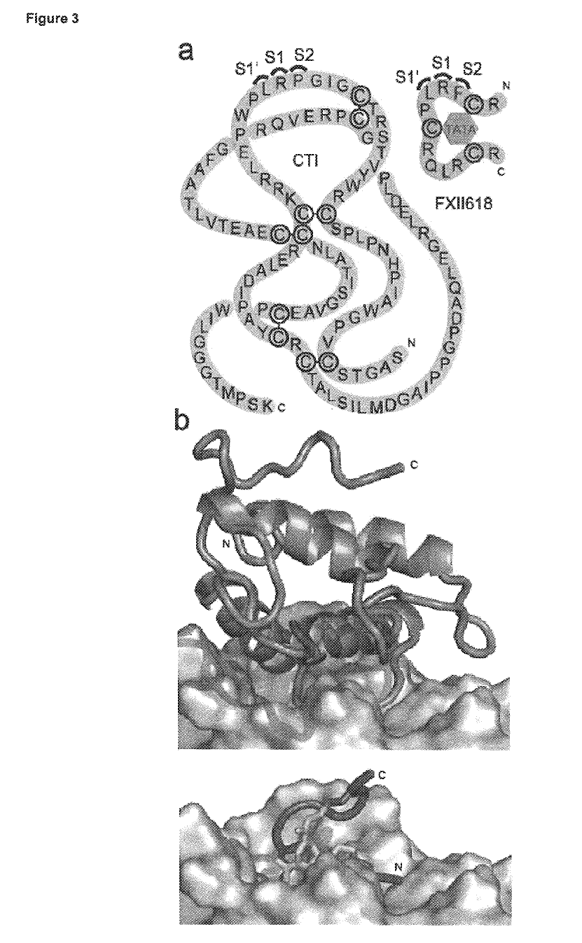

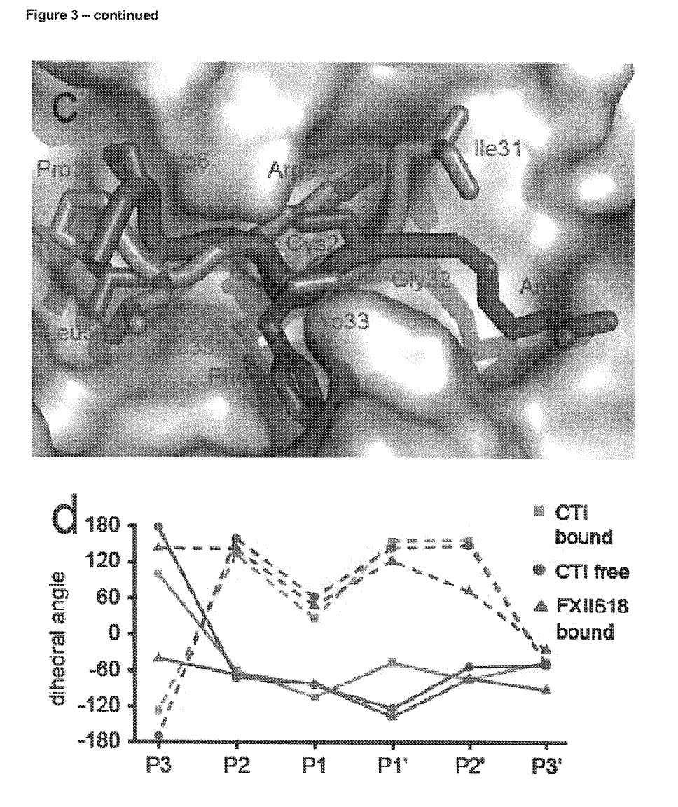

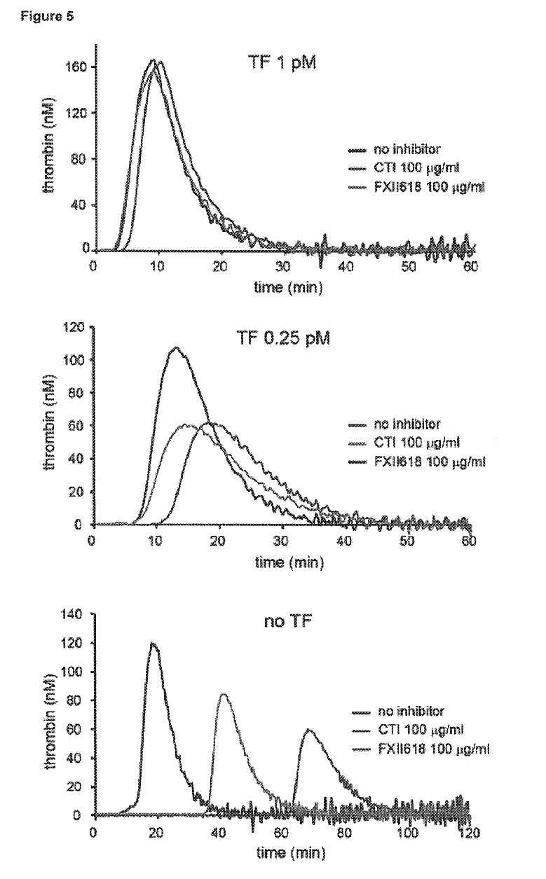

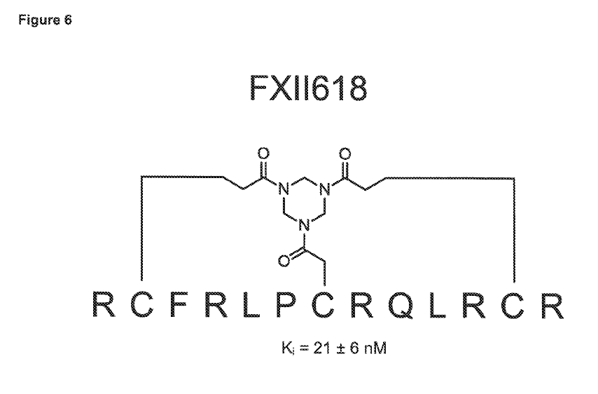

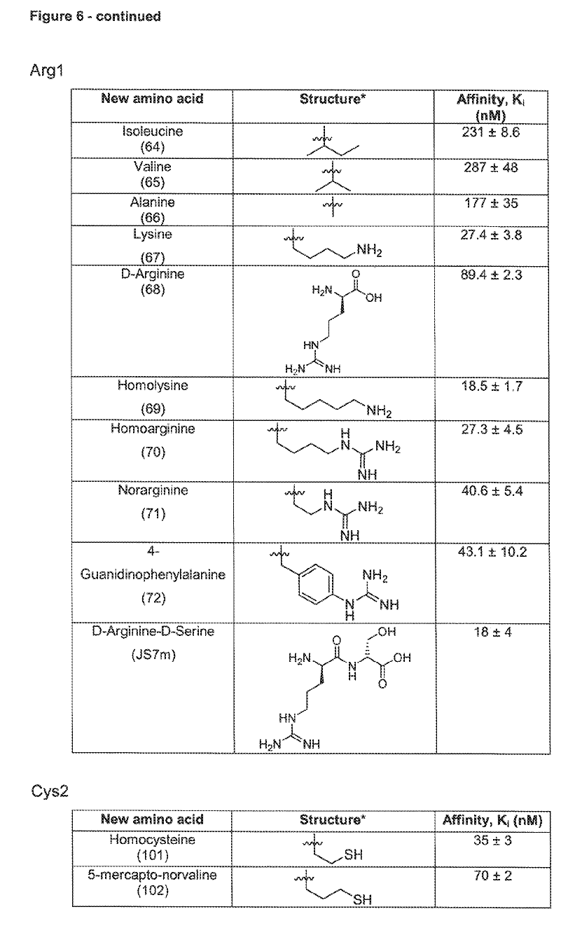

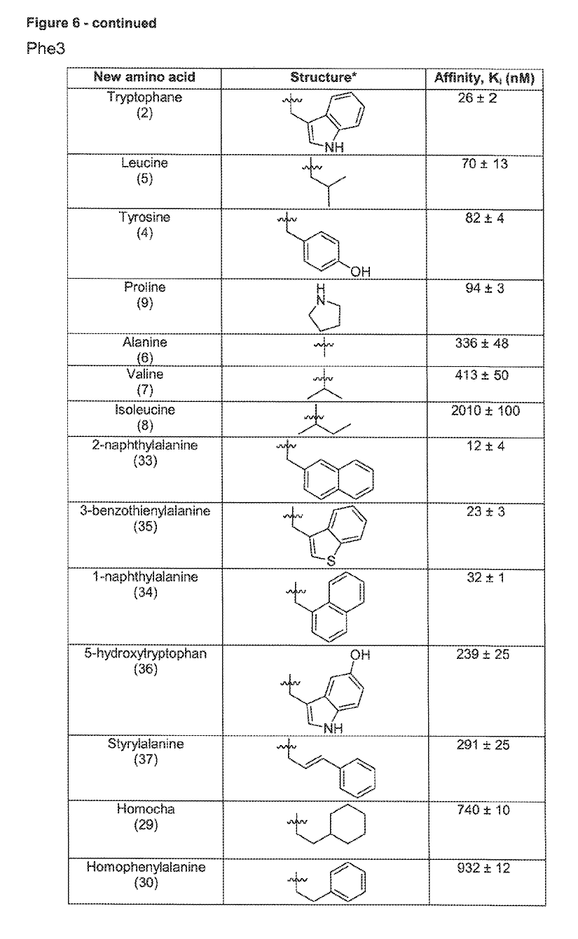

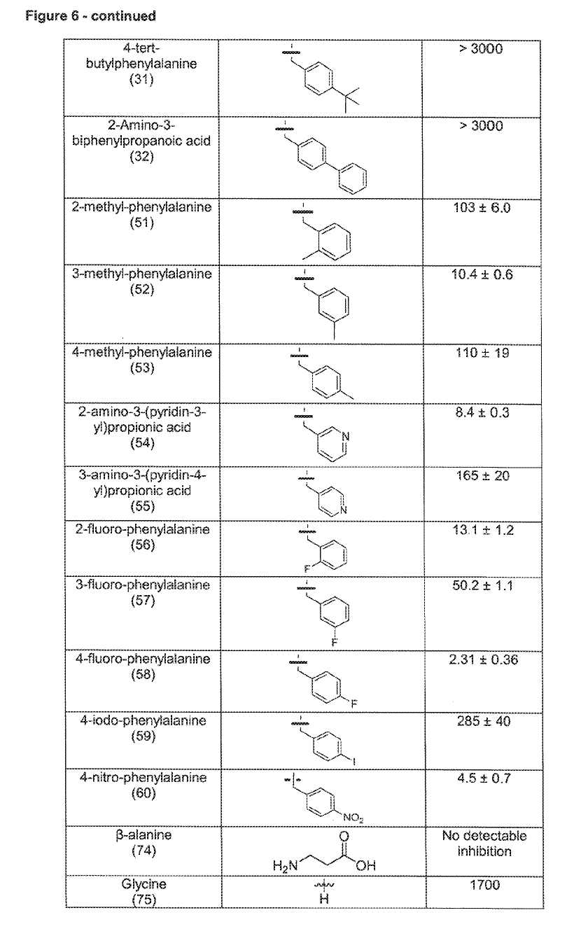

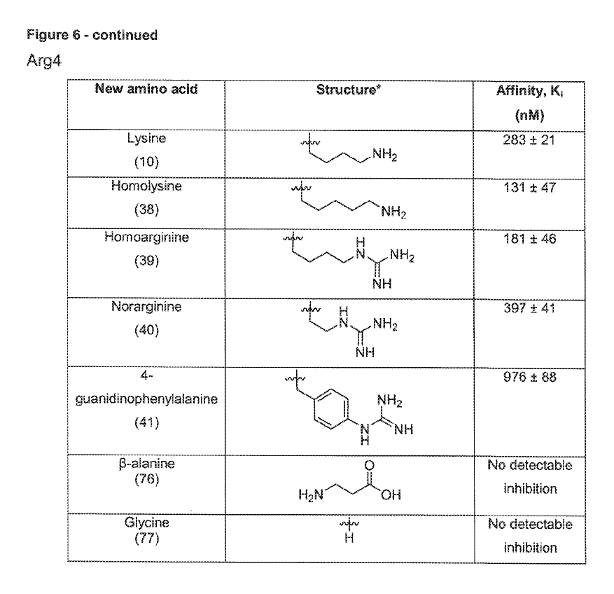

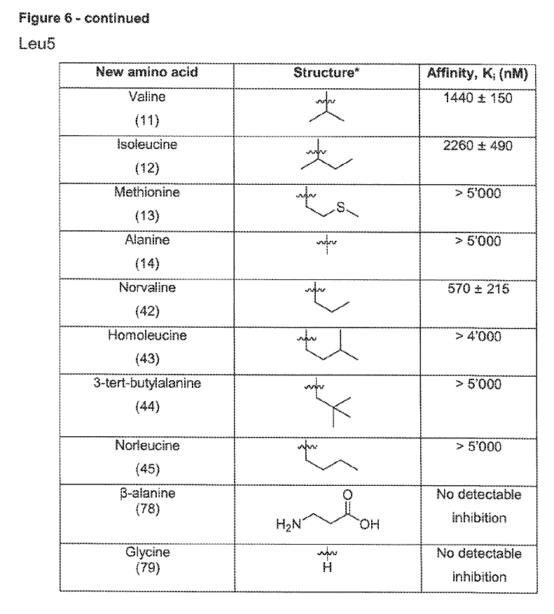

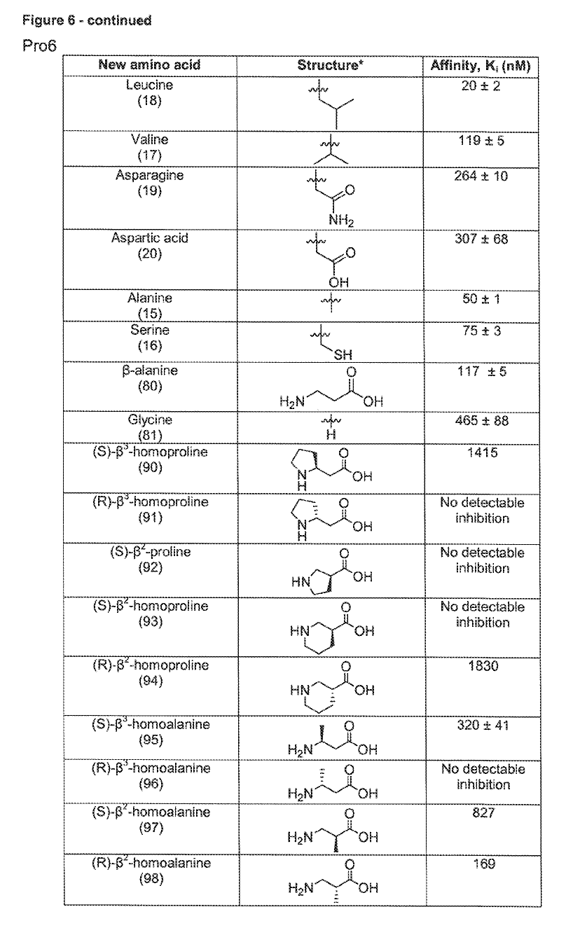

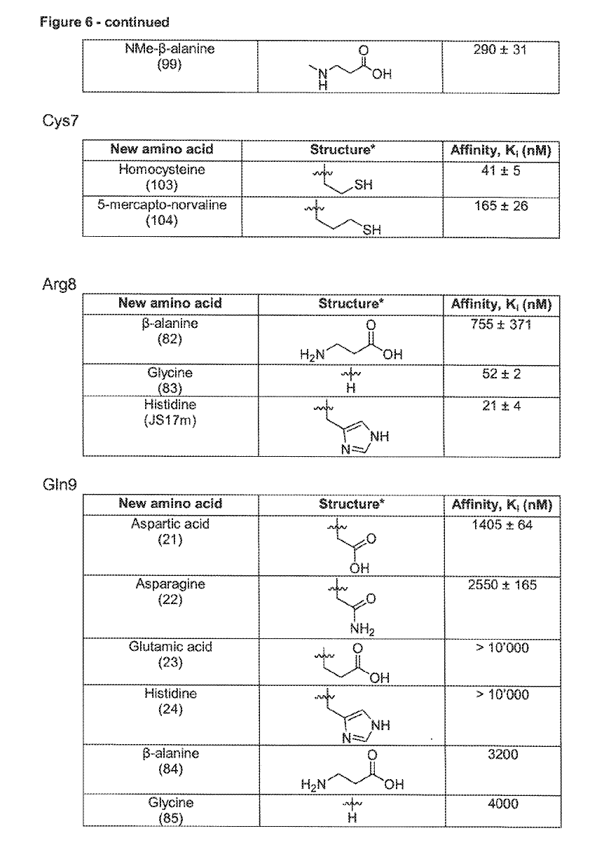

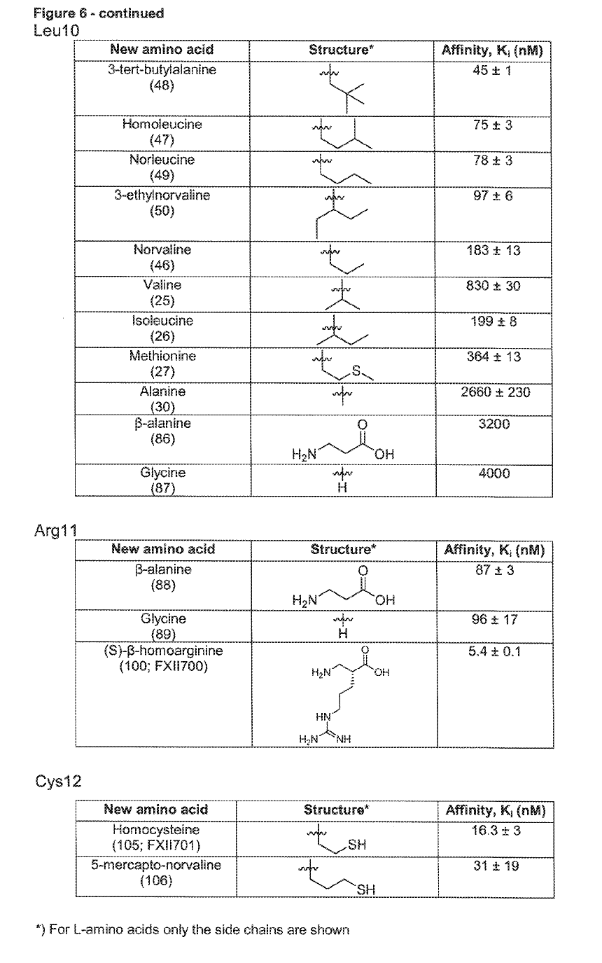

The corn trypsin inhibitor (CTI) is the current gold-standard of a coagulation inhibitor in coagulation assays. Addition of CTI at the moment of sample collection prevents contact activation of the coagulation pathway during subsequent sample processing steps. Sequence analysis and homology modelling surprisingly revealed that the first macrocyclic ring of the bicyclic peptide FXII618 structurally resembles the combining loop of the (CTI), noting that said loop mediates the inhibitory activity of CTI against FXIIa (see FIG. 3). In this respect it of note that no crystal structure of CTI (in its bound state) was available from the prior art (see Example 4). Hence, how CTI binds to FXIIa was not fully characterized. In view of this unexpected structural resemblance it is believed that the first macrocyclic ring of FXII618 predominately conveys the inhibitory capability of FXII618 against FXIIa while for the second macrocyclic ring of FXII618 no interactions with the specificity pocket of FXIIa have been found (see also snug fit of the first ring into the FXIIa active site in FIG. 3). In order to further determine which amino acids in the first macrocyclic ring of FXII618 predominately convey the inhibitory capability of FXII618 against FXIIa, amino acid changes were introduced into the ring and thereafter the Ki of the mutated inhibitors determined (see FIG. 6). It was found that in the first macrocyclic ring the dipeptide RL is essential and may not be changed without considerably diminishing the inhibitory capability. On the other hand, it has been established that the other amino acids within the first macrocyclic ring may be changed. The generic peptide sequence set forth in the embodiment constituting the first aspect of the invention is therefore based on the structure of the first macrocyclic ring of the bicyclic peptide FXII618. With regard to the amino acids in positions (X³)ₙ and (X⁶)ₘ it noted that n and m are 0 in the first ring of the bicyclic peptide FXII618. In other terms, these amino acids are absent in FXII618. While it is believed that further amino acids may be added into the first ring without substantially limiting the inhibitory activity against FXIIa it is most preferred that n and m are 0, as in FXII618.

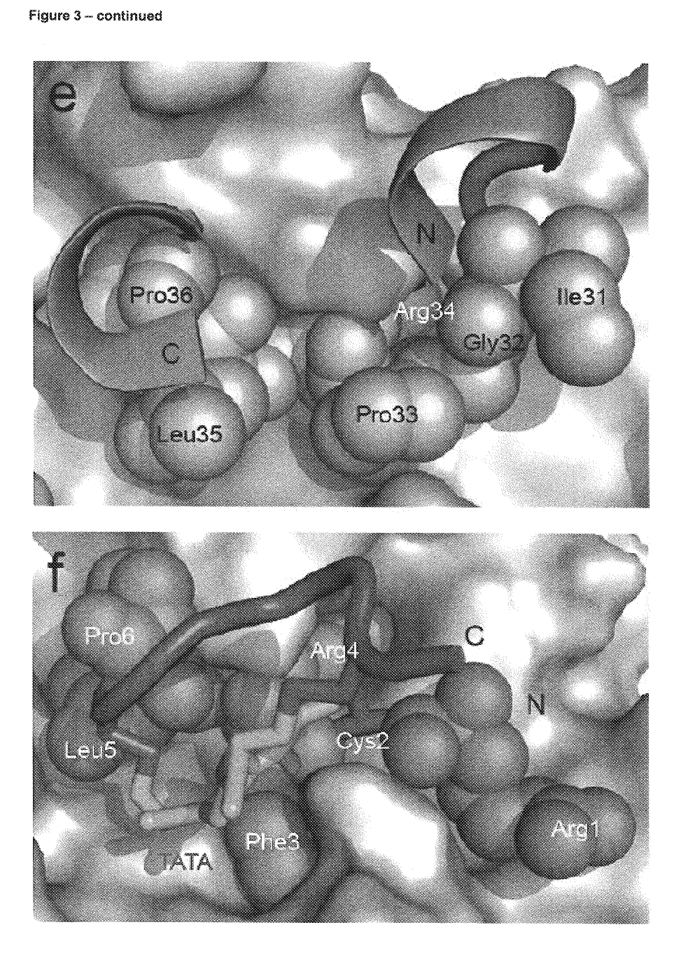

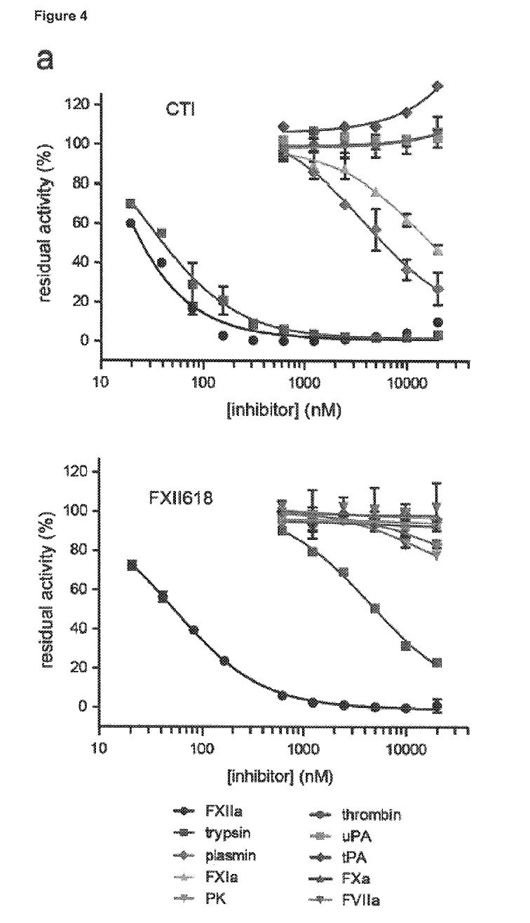

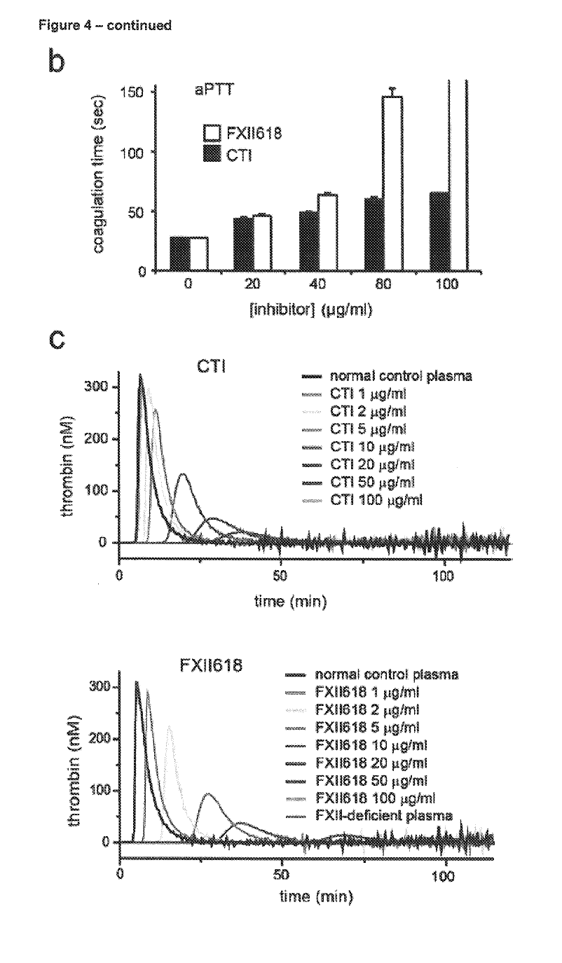

As demonstrated in the examples herein below, FXII618 is more potent and selective than CTI (see FIG. 4) in repressing the intrinsic pathway of coagulation and it has a better specificity profile. In addition, FXII618 is a cost-efficient alternative to the currently applied gold-standard CTI. On a gram scale, production costs for CTI are around 20,000 dollar per gram. In contrast, 1 gram bicyclic peptide FXII618 costs around 2,000 dollar and thus about 10 times less. Moreover, CTI needs to be purified from corn seeds chromatographically. The small peptide FXII618 can advantageously readily be ordered from peptide synthesis companies and may also be made available as a commercial research reagent. FXII618 is therefore a very attractive alternative to CTI in the diagnosis tests discussed above and a promising candidate for the control of FXII activity in coagulation and plasma kallikrein-induced inflammation disorders. FXII618 thus has a strong potential to be broadly used both in research and routine labs. In view of the advantageous properties of FXII618 over CTI, FXII818 is expected to replace CTI in several coagulation assays.

In view of the advantageous properties of FXII618 it is also preferred that the cyclic inhibitor of the first aspect of invention as well as any preferred form thereof described herein below comprises or consists of a peptide which differs with increasing preference by no more than three, two and one amino acid(s) changes from SEQ ID NO: 1. Amino acids changes may be substitutions, deletions or additions of amino acids and are preferably substitutions.

In accordance with a preferred embodiment of the first aspect of the invention, the inhibitor has an inhibitory constant (Kᵢ) for FXIIa of less than 500 nM, preferably less than 250 nM, more preferably less than 100 nM, even more preferably less than 50 nM and most preferably less than 25 nM.

The inhibitory constant (Kᵢ) is a well know measure in the field of enzyme inhibition. The Kᵢ is an indication of how potent an inhibitor is; it is the concentration required to produce the half maximum inhibition. Whereas the IC₅₀ value for a compound may vary between experiments, the Kᵢ is an absolute value. The calculation of the Kᵢ on the basis of the Cheng-Prusoff equation is further explained in the examples herein below. As discussed herein above the Ki of FXII618 for FXIIa is 22 nM and the Kᵢ of CTI for FXIIa is 24 nM. Hence, the most preferred Ki of less than 25 nM for FXIIa refers to inhibitors which at least as good as FXII618 and CTI. Also in this respect to the Kᵢ values FXIIa is most preferably human α-FXIIa or β-FXIIa.

In accordance with a further preferred embodiment of the first aspect of the invention, the inhibitor specifically inhibits FXIIa.

The term “the inhibitor specifically inhibits FXIIa” means that the inhibitor does not substantially inhibit related proteases; in particular not those proteases that should not be inhibited in the respective application of the FXIIa inhibitor (e.g. in therapeutic applications, physiologically important proteases are not substantially inhibited). The inhibitor does not inhibit these proteases with increasing preference at concentration of as high as 100 nM, 250 nM, 500 nM and 1000 nM. The term “related proteases” preferably refers to proteases comprising or consisting of trypsin and more preferably in addition to trosin, thrombin, plasmin, FXIa, PK (plasma kallikrein), uPA (urokinase-type plasminogen activator), tPA (tissue-type plasminogen activator), FXa and FVIIa. As demonstrated in the examples herein below, CTI significantly inhibits the activity of trypsin already at a concentration as low as 20 nM while for FXII618 no inhibition of trypsin is observed at a concentration as high as 1000 nM (see FIG. 4).

In accordance with a still further preferred embodiment of the first aspect of the invention, the amino acid (X⁴) is preferably an aromatic L-amino acid, wherein the alpha-carbon of the aromatic L-amino acid is attached to the aromatic ring structure by a single CH₂ group, and most preferably an aromatic L-amino acid, wherein the alpha-carbon of the aromatic L-amino acid is attached to the aromatic ring structure by a single CH₂ group and wherein the ring structure of the an aromatic L-amino acid has no other substituents.

As can be taken from the Table in FIG. 6 in case the amino acid F in position three of the peptide FXII618 is replaced by an amino acid having the structural characteristics according to this preferred embodiment the inhibitory capacity of FXIIa remains largely unaffected or is even increased.

In accordance with a yet further preferred embodiment of the first aspect of the invention, the amino acid (X⁴) is selected from the group consisting of L, P, F, W, Y, 1-naphthylalanine, 2-naphthylalanine, 3-benzothienylalanine, 3-fluoro-phenylalanine, 3-methyl-phenylalanine, 2-amino-3-(pyridin-3-yl)propionic acid, 2-fluoro-phenylalanine, 4-fluoro-phenylalanine, and 2-nitro-phenylalanine, preferably the group consisting of F, W, 1-naphthylalanine, 2-naphthylalanine, 3-benzothienylalanine, 3-methyl-phenylalanine, 2-amino-3-(pyridin-3-yl)propionic acid, 2-fluoro-phenylalanine, 4-fluoro-phenylalanine, and 2-nitro-phenylalanine, more preferably the group consisting of F, W, 2-naphthylalanine, 3-benzothienylalanine, 3-methyl-phenylalanine, 2-amino-3-(pyridin-3-yl)propionic acid, 2-fluoro-phenylalanine, 4-fluoro-phenylalanine, and 2-nitro-phenylalanine, and most preferably the group consisting of 2-naphthylalanine, 3-methyl-phenylalanine, 2-amino-3-(pyridin-3-yl)propionic acid, 2-fluoro-phenylalanine, 4-fluoro-phenylalanine, and 2-nitro-phenylalanine.

The amino acid F in position three of the peptide FXII618 corresponds to the amino acid (X⁴). As can be taken from the Table in FIG. 6, in case the amino acid F in position three of the peptide FXII618 is replaced by L, P, F, W, Y, 1-naphthylalanine, 2-naphthylalanine, 3-bentothienylalanine, 3-fluoro-phenylalanine, 3-methyl-phenylalanine, 2-amino-3-(pyridin-3-yl)propionic acid, 2-fluoro-phenylalanine, 4-fluoro-phenylalanine or 2-nitro-phenylalanine a Kᵢ of below 100 nM is maintained in all cases. In case of a replacement by F, W, 1-naphthylalanine, 2-naphthylalanine, 3-benzothienylalanine, 3-methyl-phenylalanine, 2-amino-3-(pyridin-3-yl)propionic acid, 2-fluoro-phenylalanine, 4-fluoro-phenylalanine, or 2-nitro-phenylalanine a Kᵢ of below 50 nM is maintained. The replacement by F, W, 2-naphthylalanine, 3-benzothienylalanine, 3-methyl-phenylalanine, 2-amino-3-(pyridin-3-yl)propionic acid, 2-fluoro-phenylalanine, 4-fluoro-phenylalanine or 2-nitro-phenylalanine results in a K_{I} being essentially at least as good as the Ki of FXII618 or CM In case F is replaced by 2-naphthylalanine, 3-methyl-phenylalanine, 2-amino-3-(pyridin-3-yl)propionic acid, 2-fluoro-phenylalanine, 4-fluoro-phenylalanine or 2-nitro-phenylalanine the Kᵢ is even remarkably improved to below 15 nM in all instances.

In accordance with a further preferred embodiment of the first aspect of the invention, the amino acid (X⁵) is a hydrophobic L-amino acid or a polar, uncharged L-amino acid, more preferably a hydrophobic L-amino acid, even more preferably S, A, L or P and most preferably L or P.

The amino acid P in position six of the peptide FXII618 corresponds to the amino acid (X⁵). As can be taken from the Table in FIG. 6 in case the amino acid P in position six is replaced by another hydrophobic L-amino acid or a polar, uncharged L-amino acid the inhibitory capacity is not significantly reduced. Replacement by another hydrophobic L-amino acid largely maintained the inhibitory capacity, while replacement by L even slightly improved the inhibitory capacity. For example, it is shown in FIG. 6 that in case P is replaced by S, A or L the Ki for FXIIa is in all instances below 100 nM.

In accordance with another preferred embodiment of the first aspect of the invention, the side chains of (X²) and (X⁷) comprise a functional group, preferably for each of (X²) and (X⁷) independently selected from —NH₂—COOH, —OH, —SH, alkene, alkyne, azide and chloroacetamide, more preferably —NH₂ and —SH, and most preferably —SH.

The at least two functional groups of the connecting molecule may be the same or different and are preferably the same. In the peptide the FXII618 amino acids at position 2 and 7 correspond to the amino acids (X²) and (X⁷). It is believed that the exact structure of the functional group within the side chain of the amino acid is not particularly critical as long as it allows for the formation of a ring via the connecting molecule. The functional groups —NH₂—COOH, —OH, —SH, alkene, alkyne, azide and chloroacetamide are preferred as in particular for these functional groups the required chemical reactions to form a ring are well-established in the art. —SH is most preferred as in FXII618 the amino acids at position 2 and 7 are both cysteine.

In accordance with a more preferred embodiment of the first aspect of the invention, the amino acids (X²) and (X⁷) are each independently K, ornithine, thialysine, 2,3-diaminopropanoic acid, diaminobutyric acid, D, E, C, homocysteine, penicillamine and propargylglycine, preferably C or homocysteine and most preferably both are C.

The two amino acids (X²) and (X⁷) may be the same or different and are preferably the same. The amino acids listed in this more preferred embodiment carry in their side chains a functional group selected from —NH₂—COOH, —OH, —SH, alkene, alkyne, azide and chloroacetamide. Cysteine is most preferred as in FXII618 the amino acids at position 2 and 7 are both cysteine.

In accordance with a still further preferred embodiment of the first aspect of the invention, (X²) is 5-mercapto norvaline, homocysteine or C, preferably homocysteine or C and is most preferably C; and/or (X⁷) is homocysteine or C, preferably C.

As mentioned, the amino acids at positions 2 and 7 in FXII618 are both C. As is evident from FIG. 6, C may be replaced in amino acid position 2 and/or 7 by another amino acid without significantly reducing the inhibitory capacity vis-à-vis FXIIa. For example, in case C in (X²) is replaced by 5-mercapto norvaline or homocysteine the Ki for FXIIa is still below 100 nM. In case C in (X⁷) is replaced by homocysteine the Ki for FXIIa is still below 50 nM.

In accordance with another preferred embodiment of the first aspect of the invention, (X¹) and (X⁸) are present and are each independently an aliphatic L-amino acid or polar, basic L-amino acid; preferably a polar, basic L-amino acid with an amine group; more preferably (X¹) and (X⁸) are each independently selected from lysin, homolysin, arginine, homoarginine and are most preferably both are arginine.

The amino acids (X¹) and (X⁸) may be the same or different and are preferably the same. In the peptide FXII618 amino acids at position 1 and 8 correspond to the amino acids (X¹) and (X⁸). It is believed that the exact nature of the amino acid in positions (X¹) and (X⁸) is not particularly critical. Because in the peptide FXII618 amino acids at positions 1 and 8 are R, the preferred amino acids for (X¹) and (X⁸) in accordance with the above preferred embodiment are amino acids which structurally resemble R and are preferably R.

In accordance with a further preferred embodiment of the first aspect of the invention, (X¹) is D-Arg, homoarginine, L, norarginine, 4-guanidinophenylalanine, homolysine, D-Arg-D-Ser or L-Arg, preferably homoarginine, L, norarginine, 4-guanidinophenylalanine, homolysine, D-Arg-D-Ser or L-Arg, and most preferably homolysine, D-Arg-D-Ser or R; and/or (X⁸) is G, H or R, preferably H or R.

As mentioned, the amino acids at positions 1 and 8 in FXII618 are both R. As is evident from FIG. 6, R may be replaced in amino acid position 1 and/or 8 by another amino acid without significantly reducing the inhibitory capacity vis-à-vis FXIIa. For example, in case R in (X¹) is replaced by homolysine or D-Arg-D-Ser the Ki for FXIIa is at least as good as the Ki for FXIIa of FXII618. Also in case R in (X⁸) is replaced by H the Ki for FXIIa is at least as good as the Ki for FXIIa of FXII618.

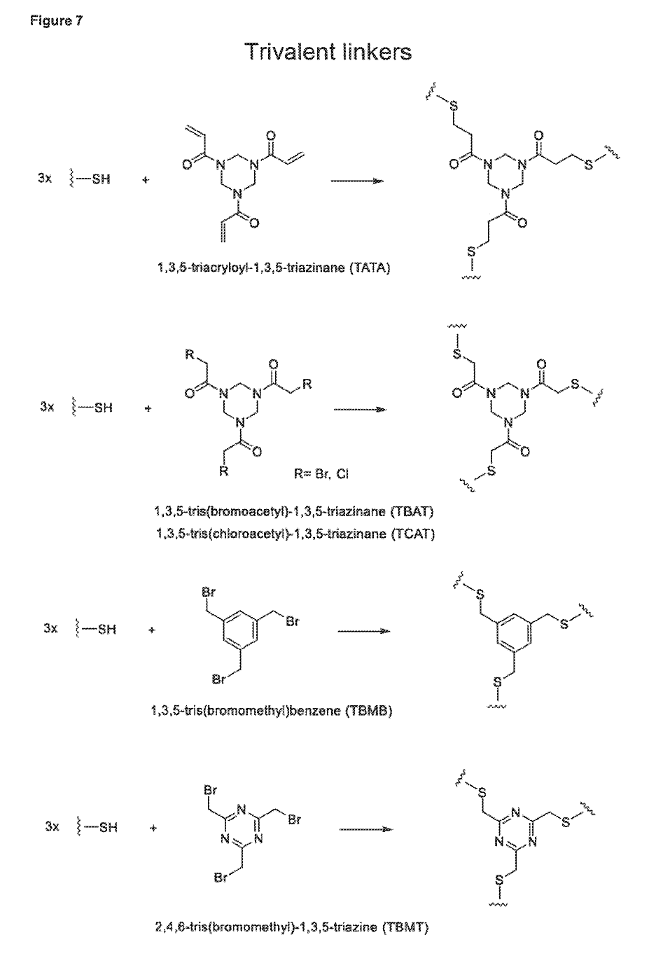

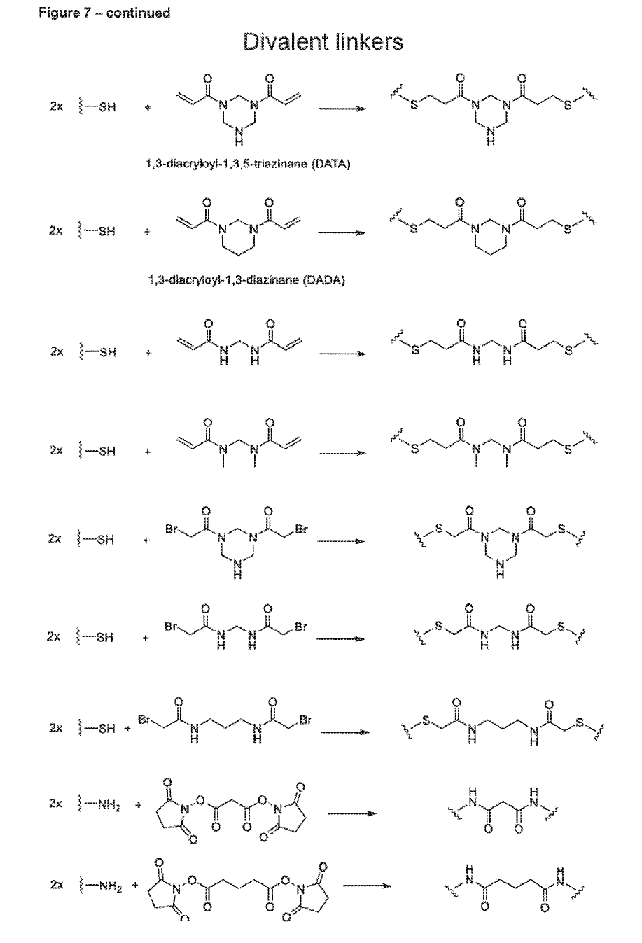

In accordance with a yet further preferred embodiment of the first aspect of the invention, the connecting molecule is selected from the trivalent and divalent linkers shown in FIG. 7 of the application, and is most preferably 1,3-diacryloyl-1,3,5-triazinane (DATA), 1,3-diacryloyl-1,3-diazinane (DADA), or 1,3,5-triacryloyl-1,3,5-triazinane (TATA).

In the peptide FXII618 the connecting molecule is TATA. As the above preferred embodiment only comprises the first ring of FXII618 it is evident that TATA can be replaced by DATA or DADA. Two functional groups are sufficient to connect the amino acids (X²) and (X⁷) as to form a ring. It appears that the exact nature of the connecting molecule is not particularly critical. This is because the ring structure itself does not from interactions with FXIIa but is to bring the amino acids in the peptide into a 3D-conformation wherein the peptide can ideally interact with FXIIa, said interaction resulting in the inhibition of FXIIa. It follows that any linker capable of bringing the amino acids into a 3D-conformation which at least resembles the 3D-conformation induced by TATA is expected to be a suitable connecting molecule in the present invention.

In accordance with a further preferred embodiment of the first aspect of the invention, the amino acid (X⁸) is absent and the inhibitor is a bicyclic inhibitor comprising or consisting of the peptide (X¹)(X²)(X³)ₙ(X⁴)RL(X⁵)(X⁶)ₘ(X⁷)(X⁹)ₗ(X¹⁰)(X¹¹)(X¹²)(X¹³)(X¹⁴)ₖ(X¹⁵)(X¹⁶), wherein (X¹) to (X⁷), n and m are as defined herein above; (X⁹) is an amino acid and l is between 0 and 3, preferably 0 or 1 and most preferably 0; (X¹⁰) is an amino acid; (X¹¹) is an amino acid, preferably Q; (X¹²) is a hydrophobic L-amino acid, preferably an aliphatic L-amino acid, and is most preferably L; (X¹³) is an amino acid; (X¹⁴) is an amino acid and k is between 0 and 3, preferably 0 or 1 and most preferably 0, (X¹⁵) is an amino acid with a side chain; and (X¹⁶) is present or absent and, if present, is an amino acid; and wherein the side chains of (X²), (X⁷) and (X¹⁵) are connected via a connecting molecule, said connecting molecule having at least three functional groups, each functional group forming a covalent bond with one of the side chains of (X²), (X⁷) and (X¹⁵).

Hence, the present invention relates in a second aspect to a bicyclic inhibitor of the coagulation enzyme activated factor XII (FXIIa) comprising or consisting of the peptide (X¹)(X²)(X³)ₙ(X⁴)RL(X⁵)(X⁶)ₘ(X⁷)(X⁹)ₗ(X¹⁰)(X¹¹)(X¹²)(X¹³)(X¹⁴)ₖ(X¹⁵)(X¹⁶), wherein (X¹) is present or absent and, if present, is an amino acid; (X²) is an amino acid with a side chain; (X³) is an amino acid and n is between 0 and 3, preferably 0 or 1 and most preferably 0; (X⁴) is an aliphatic L-amino acid or a cyclic L-amino acid, preferably L, P or an aromatic L-amino acid, and most preferably an aromatic L-amino acid; (X⁵) is an amino acid; (X⁶) is an amino acid and m is between 0 and 3, preferably 0 or 1 and most preferably 0; (X⁷) is an amino acid with a side chain; (X⁹) is an amino acid and l is between 0 and 3, preferably 0 or 1 and most preferably 0; (X¹⁹) is an amino acid; (X¹¹) is an amino acid, preferably Q; (X¹²) is a hydrophobic Is-amino acid, preferably an aliphatic L-amino acid, and is most preferably L; (X¹³) is an amino acid; (X¹⁴) is an amino acid and k is between 0 and 3, preferably 0 or 1 and most preferably 0, (X¹⁵) is an amino acid with a side chain; and (X¹⁶) is present or absent and, if present, is an amino acid; and wherein the side chains of (X²), (X⁷) and (X¹⁵) are connected via a connecting molecule, said connecting molecule having at least three functional groups, each functional group forming a covalent bond with one of the side chains of (X²), (X⁷) and (X¹⁵).

The definitions and preferred embodiments set forth herein above, where applicable, in connection with the first aspect of the invention equally apply to the bicyclic inhibitor of the invention. For instance, also the bicyclic inhibitor may have a inhibitory constant (Kᵢ) for FXIIa of less than 500 nM, preferably less than 250 nM, more preferably less than 100 nM, even more preferably less than 50 nM and most preferably less than 25 nM. Likewise this inhibitor preferably specifically inhibits FXIIa.

As discussed herein above the peptide FXII618 (SEQ NO: 1) is a bicyclic inhibitor of FXIIa. The generic peptide sequence set forth in the above preferred embodiment of the first aspect of the invention as well as the peptide sequence set forth of the second aspect of the invention is based on the structure of both macrocyclic rings of the bicyclic peptide FXII618. In the generic peptide sequence the first ring is formed between (X²) and (X⁷) while the second ring is formed between (X⁷) and (X¹⁵).

With regard to the amino acids in positions (X³)ₙ and (X⁶)ₘ, (X⁹)ₗ and (X¹⁴)ₖ it is noted that n, m, l and k are all 0 in the rings of the bicyclic peptide FXII618. In other terms; these amino acids are absent in FXII618. While it is believed that further amino acids may be added into the first ring without substantially limiting the inhibitory activity against FXIIa, it is most preferred that n, m, l and k are all 0, as in FXII618.

As it is shown in the examples herein below the second cycle does most likely not form any specific interactions with FXIIa. For this reason none of the amino acids in the broadest sense of the above preferred embodiment is fixed for the second cycle. The amino acids in positions 9 and 10 of FXII618 correspond to the amino acids (X¹¹) and (X¹²) of the generic peptide sequence of the above preferred embodiment. As is evident from the amino acid changes of Gln9 and Leu10 shown in FIG. 6 a certain preference may be given to certain amino acids in the corresponding positions (X¹¹) and (X¹²). For this reason (X^{1′}) is preferably Q and (X¹²) is an aliphatic L-amino acid, is preferably homoleucine, norleucine, 3-ethylnorvaline, 3-tert-butylalanine or L, is more preferably 3-tert-butylalanine or L and is most preferably L. These amino acids in the second cycle possibly contribute to bring the peptide FXII618 into a 3D conformation which is ideal for the inhibition of FXIIa.

In accordance with a preferred embodiment of the second aspect of the invention (X⁴) is selected from the group consisting of L, P, F, W, Y, 1-naphthylalanine, 2-naphthylalanine, 3-benzothienylalanine, 3-fluoro-phenylalanine, 3-methyl-phenylalanine, 2-amino-3-(pyridine-3-yl)propionic acid, 2-fluoro-phenylalanine, 4-fluoro-phenylalanine, and 2-nitro-phenylalanine, preferably the group consisting of F, W, 1-naphthylalanine, 2-naphthylalanine, 3-benzothienylalanine, 3-methyl-phenylalanine, 2-amino-3-(pyridin-3-yl)propionic acid, 2-fluoro-phenylalanine, 4-fluoro-phenylalanine, and 2-nitro-phenylalanine, more preferably the group consisting of F, W, 2-naphthylalanine, 3-benzothienylalanine, 3-methyl-phenylalanine, 2-amino-3-(pyridin-3-yl)propionic acid, 2-fluoro-phenylalanine, 4-fluoro-phenylalanine, and 2-nitro-phenylalanine, and most preferably the group consisting of 2-naphthylalanine, 3-methyl-phenylalanine, 2-amino-3-(pyridin-3-yl)propionic acid, 2-fluoro-phenylalanine, 4-fluoro-phenylalanine, and 2-nitro-phenylalanine.

In accordance with a further preferred embodiment of the second aspect of the invention (X⁵) is a hydrophobic L-amino acid or a polar, uncharged L-amino acid, preferably a hydrophobic L-amino acid, more preferably S, A, L or P and most preferably L or P.

In accordance with a more preferred embodiment of the first aspect of the invention as well as a preferred embodiment of the second aspect of the invention, the side chains of (X²), (X⁷) and (X¹⁵) comprise a functional group, preferably for each of (X²), (X⁷) and (X¹⁵) independently selected from NH₂, —COOH, —SH, alkene, alkyne, azide and chloroacetamide, preferably —NH₂ and —SH, and most preferably —SH.

The at least three functional groups of the connecting molecule may be the same or different and are preferably the same. In the peptide FXII618 amino acids at position 2, 7 and 12 correspond to the amino acids (X²), (X⁷) and (X¹⁵). It appears that the exact structure of the functional group within the side chain of the amino acid is not particularly critical as long as it allows for the formation of a ring via the connecting molecule, the functional groups —NH₂, —COOH, —OH, —SH, alkene, alkyne, azide and chloroacetamide are preferred as in particular for these functional groups the required chemical reactions are well-established in the art, —SH is most preferred as in FXII618 the amino acids at position 2, 7 and 12 are all cysteine.

In accordance with another more preferred embodiment of the first aspect of the invention as well as a preferred embodiment of the second aspect of the invention, the amino acids (X²), (X⁷) and (X¹⁵) are each independently K, ornithine, thialysine, 2,3-diaminopropanoic acid, diaminobutyric acid, D, E, C, homocysteine, penicillamine or propargylglycine, preferably C or homocysteine and most preferably are all C.

The three amino acids (X²), (X⁷) and (X¹⁵) may be the same or different and are preferably the same. The amino acids listed in this more preferred embodiment carry in their side chains one functional group selected from —NH₂, —COOH, —OH, —SH, alkene, alkyne, azide and chloroacetamide. Cysteine for amino acids (X²), (X⁷) and (X¹⁵) is most preferred as in FXII618 the amino acids at position 2, 7 and 12 are all cysteine.

In accordance with another more preferred embodiment of the first aspect of the invention as weal as a preferred embodiment of the second aspect of the invention, (X²) is 5-mercapto-norvaline, homocysteine or C, preferably homocysteine or C and is most preferably C; and/or (X⁷) is homocysteine or C, preferably C; and/or (X¹⁵) is 5-mercapto-norvaline, homocysteine or C, preferably homocysteine or C.

The amino acids at positions 2, 7 and 12 of FXII618 are all C. (X²), (X⁷) and (X¹⁵) in the above more preferred embodiment correspond to amino acid positions 2, 7 and 12 in FXII618, respectively. As is furthermore evident from FIG. 6, C may be replaced in amino acid position 2, 7 and/or 15 by another amino acid without significantly reducing the inhibitory capacity vis-à-vis FXIIa. For example, in case C in (X¹⁵) is replaced by homocysteine the Ki for FXIIa is 16 nM and thus even better than the Ki for FXIIa of FXII618.

In accordance with a further preferred embodiment of the first aspect of the invention as well as a preferred embodiment of the second aspect of the invention, the amino acids (X¹⁰) and (X¹⁶) are present and are each independently an aliphatic L-amino acid or polar, basic L-amino acid; preferably a polar, basic L-amino acid with an amine group; more preferably each of (X¹⁰) and (X¹⁶) are each independently selected from lysine, homolysine, arginine, homoarginine and are most preferably both arginine.

In accordance with a more preferred embodiment of the first aspect of the invention as well as a preferred embodiment of the second aspect of the invention, the amino acids (X¹⁰) and (X¹³) are each independently an aliphatic L-amino acid or a polar, basic L-amino acid; preferably a polar, basic L-amino acid with an amine group; more preferably (X¹⁰) and (X¹³) are each independently selected from lysine, homolysine, arginine, homoarginine and are most preferably both arginine.

In the peptide FXII618 amino acids at position 8 and 11 correspond to the amino acids (X¹⁰) and (X¹³). It is believed that the exact nature of the amino acid in positions (X¹⁰) and (X¹³) is not particularly critical, because they do not interact with FXIIa. Because in the peptide FXII618 the amino acids at position 8 and 11 are R the preferred amino acids for (X¹⁰) and (X¹³) in accordance with the above preferred embodiment are amino acids which structurally resemble R.

In accordance with a yet further more preferred embodiment of the first aspect of the invention as well as a preferred embodiment of the second aspect of the invention, (X¹) is D-Arg, homoarginine, L, norarginine, 4-guanidinophenylalanine, homolysine, D-Ara-D-Ser or L-Arg, preferably homoarginine, L, norarginine, 4-guanidinophenylalanine, homolysine, D-Arg-D-Ser or L-Arg, and most preferably homolysine, D-Arg-D-Ser or L-Arg; and/or (X″) is G, H or R, preferably H or R; (X¹³) is A, G, (S)-63-homoarginine or R, preferably (S)-63-homoarginine or R, and/or (X″) is R or absent.

The amino acids at positions 1, 8, 11 and 13 of FXII618 are all R. The amino acids at positions 1, 8, 11 and 13 of FXII618 correspond to positions (X¹), (X¹⁰), (X¹³) and (X¹⁶) in the above more preferred embodiment, respectively. As is evident from FIG. 6 R may be replaced in one or more of amino acid positions 1, 8, 11 and 13 by another amino acid without significantly reducing the inhibitory capacity vis-à-vis FXIIa. For example, in case R is replaced in (X¹³) by (S)-β3-homoarginine the Ki for FXIIa even improved as compared to the Ki for FXIIa of FXII618. In case R in the position (X¹⁶) is absent (i.e. the R is deleted from FXII618) the Ki for FXIIa is essentially as good as the Ki for FXIIa of FXII618.

In accordance with another more preferred embodiment of the first aspect of the invention wherein the inhibitor is a bicyclic inhibitor as well as a preferred embodiment of the second aspect of the invention, the connecting molecule is selected from 1,3,5-triacryloyl-1,3,5-triazinane (TATA), 1,3,5-tris(chloroacetyi)-1,3,5-triazinane (TCAT), 1,3,5-tris(bromoacetyl)-1,3,5-triazinane (TBAT), 1,3,5-tris(bromomethyl)benzene (TBMB) and 2,4,6-tris(bromomethyl)-1,3,5-triazine (TBMT), and is preferably 1,3,5-triacryloyl-1,3,5-triazinane (TATA).

All connecting molecules according to this more preferred embodiment of the first aspect and preferred embodiment of the second aspect of the invention have three functional groups such that the above-discussed two rings of the bicyclic inhibitor can be formed. As mentioned, in the bicyclic peptide FXII618 the connecting molecule is TATA. For this reason TATA is most preferred. The other linkers recited in the preferred embodiment structurally resemble TATA and are therefore non-limiting examples of suitable alternatives for TATA. This has been exemplarily shown in the examples herein below for TBAT and TBMT. Despite the replacement of TATA by TBAT or TBMT in FXII618 a specific inhibitory capacity for FXIIa was maintained.

In accordance with another more preferred embodiment of the first aspect of the invention as well as a preferred embodiment of the second aspect of the invention at least one of the following items (i) to (iv) apply: (i) (X¹) is D-Arg-D-Ser, (ii) (X⁴) is 4-fluoro-phenylalanine, (iii) (X¹⁰) is H, and/or (iv) (X¹³) is (s)-β3-homoarginine: wherein (X¹⁶) is optionally absent, and the connecting molecule is preferably TATA.

The term “at least one” means with increasing preference at least two, at least three and all four.

As shown in the examples herein below and as discussed herein above, replacing amino acids in the amino acid sequence of FXII618—other than R and L at amino acid positions 4 and 5, respectively—generally result in derivatives of FXII618 having a Ki for FXIIa which is even better than the Ki for FXIIa of FXII618. Examples are the replacement amino acids D-Arg-D-Ser in (X¹), 4-fluoro-phenylalanine in (X⁴), H in (X¹⁰) and (S)-63-homoarginine in (X¹³), noting that positions (X¹), (X⁴), (X¹⁰) and (X¹³) correspond to amino acid positions 1, 4, 8 and 11 of FXII618, respectively.

Because in particular D-Arg-D-Ser in (X¹), 4-fluoro-phenylalanine in (X⁴), H in (X¹⁰) and (S)-β3-homoarginine in (X¹³) were further investigated in order to illustrate the combinatorial effect of two or more advantageous amino acid substitutions in FXII618 these amino acid substitutions are particularly preferred. It is shown in the examples herein below that in case any three or all four of these amino acid substitutions are introduced into the amino acid sequence of FXII618 the advantageous effects of each single amino acid substitution sum up and result in FXII618 derivatives which are significantly more potent than FXII618 derivatives with only one of these amino acid substitutions. In case any three or all four of these amino acid substitutions are introduced into the amino acid sequence of FXII618 the resulting FXII618 derivatives have a Ki for FXII618 which is 1 nM or even below in the pM range (see Table in Example 5.3.).

The derivatives of FXII618 shown in the Table of Example 5.3 are the most effective inhibitors against FXIIa described herein and are therefore the most preferred inhibitors of the first aspect and the second aspect of the invention. These most preferred inhibitors are rsCF^{4F}RLPCHQLR^{b}CR, rsCF^{4F}RLPCHQLRCR, rsCF^{4F}RLPCRQLR^{b}CR, rsCFRLPCHQLR^{b}CR and rsCF^{4F}RLRCHQLR^{b}C, wherein rs is D-Arg-D-Ser; F^{4F} is 4-fluoro-phenylalanine, and R^{b} is (S)-β3-homoarginine.

In the context of these combinatorial experiments it has furthermore been found that the R at the last amino acid position of the FXII618 derivatives is dispensable, noting that this dispensable amino acid corresponds to (X¹⁶). As for most FXII618 derivatives disclosed herein the connecting molecule in the combinatorial FXII618 derivatives is TATA as in FXII618.

In summary, based on the exemplified combinatorial FXII618 derivatives in Example 53 it is shown that the amino acid changes which were found in connection with the present invention to further improve the inhibitory capacity of FXII618 can be combined and generally produce in combination an improvement which goes beyond the improvement of the each individual amino acid changes.

The preferred options within the embodiment defining the first aspect and the second aspect of the invention as well as the above preferred and more preferred embodiments of the first aspect and second aspect—for each of the variant amino acid positions within the ring of the inhibitor, if present, for each the variant amino acid positions within the second ring of the inhibitor and for the connecting molecule—define structures which with increasing preference more and more resemble the structure of FXII618 and those variants thereof in FIG. 6 which retain or essentially retain the inhibitory capacity of FXII618 or even constitute an improvement. Hence, a skilled person immediately understands that embodiments incorporating any combination of the increasingly preferred amino acids for each variant amino acid position as well as optionally the preferred structures for the connecting molecule form part of the present invention.

A non-limiting example of such an embodiment is a cyclic inhibitor of the coagulation enzyme activated factor XII (FXIIa) comprising or consisting of the peptide (X¹)(X²)(X³)ₙ(X⁴)RL(X⁵)(X⁸)ₘ(X⁷)(X⁶), wherein (X¹) is an amino acid, preferably R; (X²) is C; (X³) is an amino acid and n is 0; (X⁴) is F, W, 2-naphthylalanine and 3-benzothienylalanine, and preferably 2-naphthylalanine, (X⁵) is L or P; (X⁶) is an amino acid and m is 0; (X⁷) is C; and (X⁸) is amino acid, preferably R; and wherein the side chains of (X²) and (X⁷) are connected via a connecting molecule, said connecting molecule being selected from 1,3-diacryloyl-1,3,5-triazinane (DATA), 1,3-diacryloyl-1,3-diazinane (DADA) and 1,3,5-triacryloyl-1,3,5-triazinane (TATA). Also in connection with this embodiment the inhibitor has preferably an inhibitory constant (KO for FXIIa of less than 100 nM, more preferably less than 50 nM and most preferably less than 25 nM and/or the inhibitor specifically inhibits FXIIa.

A further non-limiting example of such an embodiment is a cyclic inhibitor of the coagulation enzyme activated factor XII (FXIIa) comprising or consisting of the peptide (X¹)(X²)(X³)ₙ(X⁴)RL(X⁵)(X⁶)ₘ(X⁷)(X⁸), wherein (X¹) is an amino acid, preferably D-Arg-D-Ser or D-Arg; (X²) is C; (X³) is an amino acid and n is 0; (X⁴) is F, W, 2-naphthylalanine and 3-benzothienylalanine, 3-methyl-phenylalanine, 2-amino-3-(pyridin-3-yl)propionic acid, 2-fluoro-phenylalanine, 4-fluoro-phenylalanine, or 2-nitro-phenylalanine; and preferably is 2-naphthylalanine, 3-methyl-phenylalanine, 2-amino-3-(pyridin-3-yl)propionic acid, 2-fluoro-phenylalanine, 4-fluoro-phenylalanine, or 2-nitro-phenylalanine, (X) is L or P; (X⁸) is an amino acid and m is 0; (X⁷) is C; and (X⁸) is amino acid, preferably H or R; and wherein the side chains of (X²) and (X⁷) are connected via a connecting molecule, said connecting molecule being selected from 1,3-diacryloyl-1,3,5-triazinane (DATA), 1,3-diacryloyl-1,3-diazinane (DADA) and 1,3,5-triacryloyl-1,3,5-triazinane (TATA). Also in connection with this embodiment the inhibitor has preferably an inhibitory constant (K) for FXIIa of less than 100 nM, more preferably less than 50 nM and most preferably less than 25 nM and/or the inhibitor specifically inhibits FXIIa.

A still further non-limiting example of such an embodiment, wherein also a second ring is present, is a bicyclic inhibitor of the coagulation enzyme activated factor XII (FXIIa) comprising or consisting of the peptide (X¹)(X²)(X³)ⁿ(X⁴)RL(X⁵)(X⁶)ₘ(X⁷)(X⁹)ₗ(X¹⁰)(X¹¹)(X¹²)(X¹³)(X¹⁴)ₖ(X¹⁵)(X¹⁶), wherein (X¹) is an amino acid, preferably R; (X²) is C; (X³) is 0; (X⁴) is F, W, 2-naphthylalanine and 3-benzothienylalanine, and preferably 2-naphthylalanine, (X⁶) is L or P; (X⁶) is 0; (X⁷) is C; (X) is an amino acid and i is 0: (X¹⁶) is an amino acid, preferably R; (X¹¹) is Q; (X¹²) is L; (X′³) is an amino acid, preferably R; (X¹⁴) is an amino acid and k is 0, (X¹⁵) is C; and (X¹⁶) is an amino acid, preferably R; and wherein the side chains of (X²), (X⁷) and (X¹⁵) are connected via a connecting molecule, said connecting molecule being 1,3,5-triacryloyl-1,3,5-triazinane (TATA). Likewise in connection with this embodiment the inhibitor has preferably an inhibitory constant (1<ᵢ) for FXIIa of less than 100 nM, more preferably less than 50 nM and most preferably less than 25 nM and/or the inhibitor specifically inhibits FXIIa.

A yet further non-limiting example of such an embodiment, wherein also a second ring is present, is a bicyclic inhibitor of the coagulation enzyme activated factor XII (FXIIa) comprising or consisting of the peptide (X¹)(X²)(X⁹)ₙ(X⁴)RL(X⁵)(X⁶)ₘ(X⁷)(X⁹)ₗ(X¹⁰)(X¹¹)(X¹²)(X¹³)(X¹⁴)ₖ(X¹⁵)(X¹⁶), wherein (X¹) is an amino acid, preferably homolysine, D-Arg-D-Ser or D-Arg; (X²) is C; (X³) is an amino acid and n is 0; (X⁴) is F, W, 2-naphthylalanine and 3-benzothienylalanine, 3-methyl-phenylalanine, 2-amino-3-(pyridin-3-yl)propionic acid, 2-fluoro-phenylalanine, 4-fluoro-phenylalanine, or 2-nitro-phenylalanine; and preferably is 2-naphthylalanine, 3-methyl-phenylalanine, 2-amino-3-(pyridin-3-yl)propionic acid, 2-fluoro-phenylalanine, 4-fluoro-phenylalanine, or 2-nitro-phenylalanine, (X⁵) is L or P; (X⁶) is an amino acid and m is 0; (X⁷) is C; (X⁹) is an amino acid and l is 0; (X¹⁰) is an amino acid, preferably H or R; (X¹¹) is 0; (X¹²) is L; (X¹³) is an amino acid, preferably (S)-β-homoarginine or R; (X¹⁴) is an amino acid and k is 0, (e) is homocysteine or C; and (X¹⁶) is an amino acid, preferably absent or R; and wherein the side chains of (X²), (X⁷) and (X¹⁵) are connected via a connecting molecule, said connecting molecule being 1,3,5-triacryloyl-1,3,5-triazinane (TATA). Likewise in connection with this embodiment the inhibitor has preferably an inhibitory constant (1<ᵢ) for FXIIa of less than 100 nM, more preferably less than 50 nM and most preferably less than 25 nM and/or the inhibitor specifically inhibits FXIIa.

The present invention furthermore pertains to a cyclic inhibitor of the coagulation enzyme activated factor XII (FXIIa) comprising or consisting of the peptide (X¹)RL(X²), wherein (X¹) and (X²) are amino acids, preferably alpha-amino acids, most preferably F and P, respectively, being linked by a peptide bond; or wherein (X¹) and (X²) are amino acids with side chains and (X¹) and (X²) are connected via a connecting molecule, said connecting molecule having at least two functional groups, each functional group forming a covalent bond with one of the side chains of (X²) and (X⁷).

The definitions and preferred embodiments set forth herein above, where applicable, in connection with the first aspect and/or second aspect of the invention equally apply to this further cyclic inhibitor of the invention. Also this the inhibitor may have a inhibitory constant (Kᵢ) for FXIIa of less than 500 nN, preferably less than 250 nM, more preferably less than 100 nM, even more preferably less than 50 nM and most preferably less than 25 nM. Likewise this inhibitor preferably specifically inhibits FXIIa. An ex viva method of inhibiting the enzymatic activity of FXIIa comprises contacting the inhibitor of the invention with FXIIa, wherein FXIIa is preferably present in a blood, plasma or serum sample. In case (X¹) and (X²) are amino acids with a side chain it is preferred that the side chains comprise a functional group, preferably for each of (X¹) and (X²) independently selected from —NH₂—COOH, —OH, —SH, alkene, alkyne, azide and chloroacetamide, preferably —NH₂ and —SH, and most preferably —SH. Moreover, if (X¹) and (X²) are amino acids with side chains it is preferred that the amino acids (X¹) and (X²) are each independently K, ornithine, thialysine, 2,3-diaminopropanoic acid, diaminobutyric acid, D, E, C, homocysteine, penicillamine or propargylglycine, preferably C or homocysteine and most preferably are both C. The connecting molecule may be selected from the trivalent and divalent linkers shown in FIG. 7, and is preferably 1,3-diacryloyl-1,3,5-triazinane (DATA), 1,3-diacryloyl-1,3-diazinane (DADA), or 1,3,5-triacryloyl-1,3,5-triazinane (TATA).

The present invention in addition relates to an inhibitor of the coagulation enzyme FXIIa characterized in that it comprises at least one macrocyclic ring. Usually, said macrocyclic ring i) has a ring size of at least 12 atoms and no more than 42 atoms, and ii) contains two neighboring amino acids X₁X₂, both amino acids having an L configuration and being linked by a peptide bond wherein the side chain of the first amino acid X₁ contains a positive charge that has a distance from the alpha-carbon atom of at least four atom bonds, and wherein said inhibitor binds to the coagulation enzyme FXIIa with an inhibition constant (Ki) of less than 500 nM.

Preferably, the amino acid X₁ is selected from the group of amino acids comprising a guanidine, an amidine, a benzamidine, or an amino group in its side chain. The amino acid sequence X₁X₂ is usually flanked on each side by an amino acid and the four amino acids are linked by peptide bonds. The macrocyclic ring is based on a peptide that contains at least two cysteines that are connected via a chemical linker or a disulfide bridge.

Also preferably, said inhibitor binds to the coagulation enzyme FXIIa with an inhibition constant (Kₛ) of less than 400 nM, more preferably less than 200 nM, even more preferably less than 100 nM. Alternatively, the inhibitor of the invention comprises two macrocyclic rings that are formed by a peptide that contains at least three cysteines that are linked via the side chains to a chemical linker. Preferably, the peptide has the following sequence (SEQ ID No 1): Arg-Cys-Phe-Arg-Leu-Pro-Cys-Arg-Gln-Leu-Arg-Cys-Arg, and the chemical linker is or is based on the reagent 1,3,5-triactyloyl-1,3,5-triazinane (TATA).

Another object of the present invention concerns a bicyclic peptide inhibitor of the coagulation enzyme FXIIa consisting essentially of the amino acid sequence Xaa1-Cys-Xaa2-Xaa3-Xaa4-Cys-Xaa5-Xaa6-Xaa7-Cys-Xaa8 (SEQ ID No, 2), wherein at least three amino acids are as indicated below or independently selected from the group consisting in: Xaa2=Trp; Xaa3=Gly; Xaa4=Ala; Xaa5=Leu; Xaa6=Asn; Xaa7=Val, and wherein said bicyclic peptide inhibitor has been cyclized with 1,3,5-triacryloyl-1,3,5-triazinane.

A further object of the present invention is to provide a bicyclic peptide inhibitor of the coagulation enzyme FXIIa consisting essentially of the amino acid sequence Xaa1-Cys-Cys-Xaa2-Xaa3-Xaa4-Xaa5-Xaa6-Cys-Xaa8 (SEQ ID No. 3), wherein at least two of the amino acids are as indicated below: Xaa2=Tyr; Xaa3=Leu; Xaa4=Arg and wherein said bicyclic peptide inhibitor has been cyclized with 1,3,5-triacryloyl-1,3,5-triazinane.

Yet another object is to provide a bicyclic peptide inhibitor of the coagulation enzyme FXIIa consisting essentially of the amino acid sequence Xaa1-Cys-Xaa2-Xaa3-Xaa4-Xaa5-Xaa6-Xaa7-Cys-Xaa8-Xaa9-Xaa10-Xaa11-Xaa12-Xaa13-Cys-Xaa14 (SEQ ID No. 4) wherein at least two of the amino acids are as indicated below: Xaa2=Trp; Xaa3=Gly; Xaa4=Ala; Xaa5=Ala, and wherein said bicyclic peptide inhibitor has been cyclized with 1,3,5-triacryloyl-1,3,5-triazinane.

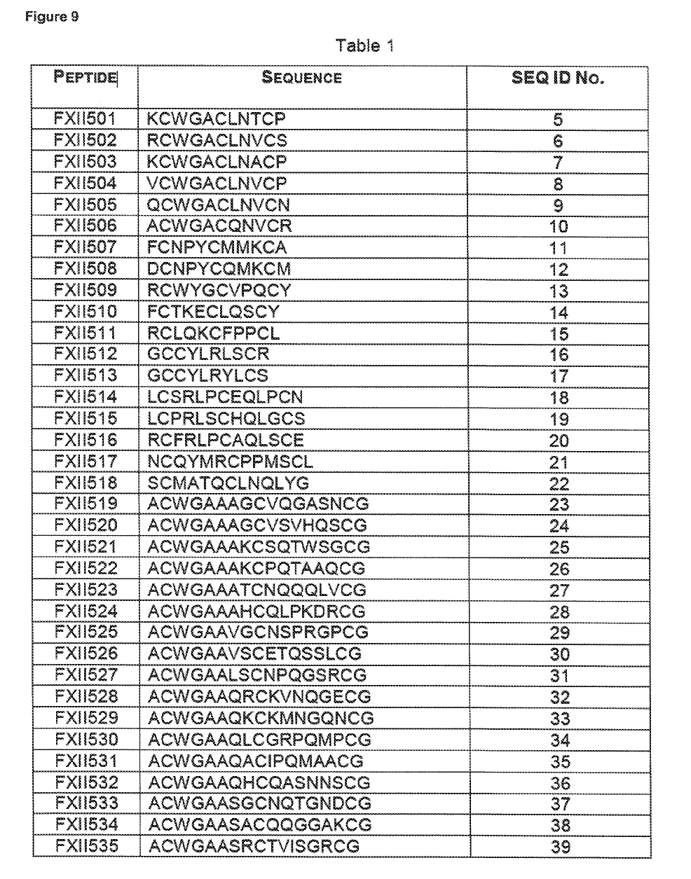

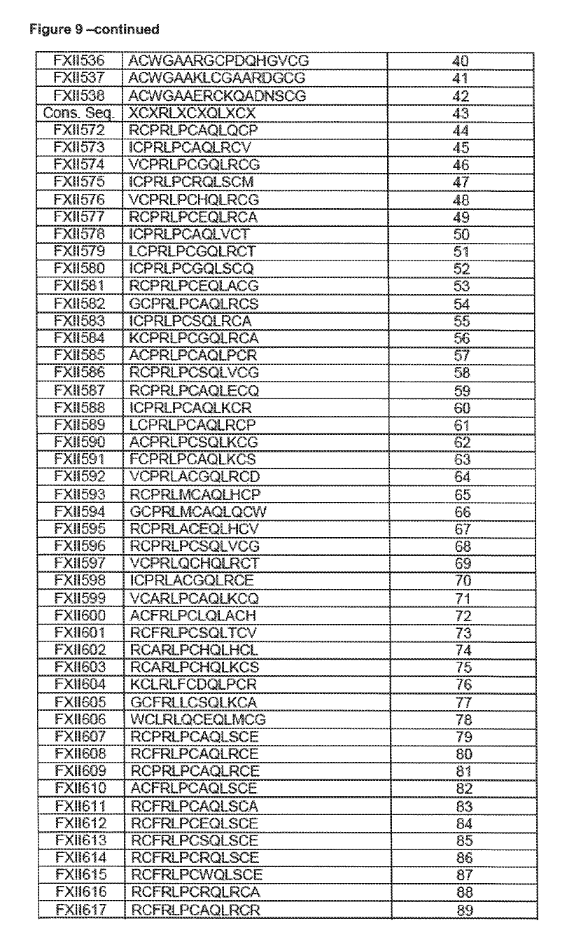

The invention also concerns an inhibitor of the coagulation enzyme FXIIa selected from the group consisting of the amino acid sequences of SEQ ID NOs 1 and 5 to 89 (see FIG. 9), as well as an amino acid at least about 90% identical to said amino acid sequences. Within SEQ ID NOs 1 and 5 to 89, SEQ ID NO: 1 is most preferred.

The invention likewise concerns an inhibitor of the coagulation enzyme FXIIa selected from the group consisting of the amino acid sequences of SEQ ID NOs 1 and 5 to 89, wherein one to several amino acids have been deleted, substituted, or added. In this respect the term one to several means with increasing preference that up to three, two or only one amino acids have been deleted, substituted, or added.

Also the inhibitor of the invention may comprise two macrocyclic rings that e formed by a peptide that contains at least three cysteines that are linked via the side chains to a chemical linker. Preferably, the peptide has the following sequence (SEQ ID NO 1): Arg-Cys-Phe-Arg-Leu-Pro-Cys-Arg-Gln-Leu-Arg-Cys-Arg, and the chemical linker is based on the reagent 1,3,5-triacryloyl-1,3,5-triazinane (TATA).

The invention furthermore relates to a fusion protein comprising the inhibitor of the invention fused to the F, domain of an antibody, an albumin binder, an IgG binder or an antibody.

The term “fusion protein” as used herein is in general terms directed to a (poly)peptide construct generated through the joining of two or more nucleic acid molecules which code for separate (poly)peptides. In other words, translation of this fused nucleic acid molecule results in a single (poly)peptide with functional properties derived from each of the original (poly)peptides. The (poly)peptides may either be directly fused or via a linker, i.e. a short peptide sequence. In general, fusion proteins are generated artificially by recombinant DNA-technology well know to the skilled person. However, fusion proteins of the invention may be prepared by any of the many conventional and well known techniques such as plain organic synthetic strategies, solid phase-assisted synthesis techniques or by commercially available automated synthesizers. Fusion proteins may be used in biological research or therapeutics.

Preferably, the Fc domain is one or more human functional Fc domains which allow(s) for extending the in vivo half-life of the inhibitor of the invention. The inhibitors of the invention can be fused either to the N- or C-terminus of one or more functional Fc domains or to both the N- and the C-terminus of one or more Fc domains. The fusion proteins of the invention may comprise multimers, such as tetramers, trimers or dimers of the inhibitors of the invention fused to at least one side, such as the N-terminus of one or more, preferably one Fc domain. Examples of an albumin binder, and an IgG binder are described in Gebauer and Skerra (2009), 13:245-255. Accordingly, preferred examples of albumin binders and an IgG binders are human single Ig domains (dubbled Albumin Dab), nanobodies, naturally occurring albumin binding domain (ABD) derived from streptococcal protein G, and domain that binds to IgG. Also such fusion proteins, for example, increase the half life of the inhibitor of the invention, in particular in the blood in vivo or ex vivo.

The invention furthermore relates to a fusion construct comprising the inhibitor of the invention fused to a pharmaceutically active compound, a diagnostically active compound and/or a component modulating serum half-life.

A “fusion construct” as used herein defines the fusion of the inhibitor of the invention to a compound, whereby the compound is selected from the group consisting of a pharmaceutically active compound, a diagnostically active compound and/or a component modulating serum half-life. The compound may either be a proteinous compound or a non-proteinous compound. In the case the compound is a proteinous compound (e.g. an antibody as described herein below), the fusion construct is also a fusion protein as defined herein above. In other words, the term “fusion constructs” comprises fusion proteins. The compound may either be directly fused to the inhibitor of the invention or via a linker. The linker according to the invention is preferably selected from the group consisting of alkyl with 1 to 30 carbon atoms, polyethylene glycol with 1 to 20 ethylene moieties, polyalanine with 1 to 20 residues, polyglycine with 1 to 20 residues, a Gly-Ser linker with 1 to 20 residues, caproic acid, substituted or unsubstituted poly-p-phenylene and triazol.

In a further preferred embodiment, said linker is selected from the group consisting of amino-n-alkyl, mercapto-n-alkyl, amino-n-alkyl-X-alkyl, mercapto-n-alkyl-X-alkyl, wherein X is selected from the group consisting of O, S—S and SO₂ and wherein the alkyl groups independently from each other have from 1 to 30 carbon atoms, preferably 3, 6 or 12 carbon atoms; or oligoethylenglycols having from one to about ten ethylenglycol moieties, preferably tri- or hexa-ethylenglycol. These and further suitable linkers are well known in the art and commercially available (see, for example, the catalogue from Glen Research, 22825 Davis Drive, Sterling, Va., 20164 USA). Further examples of linkers are the following: 5′-amino-modifiers (see e.g. B. A. Connolly and P. Rider, Nucleic Acids Res., 1985, 13, 4485; B. S. Sproat, B. S. Beijer, P. Rider, and P. Neuner, Nucleic Acids Res., 1987, 15, 4837; R. Zuckerman, D. Corey, and P. Shultz, Nucleic Acids Res., 1987, 15, 5305; P. Li, et al., Nucleic Acids Res., 1987, 15, 5275; G. B. Dreyer and P. B. Dervan, Proc. Natl. Acad. Sci. USA, 1985, 82, 968.); 5′-thiol-modifier C6 (see e.g., B. A. Connolly and P. Rider, Nucleic Acids Res., 1985, 13, 4485; B. S. Sproat, B. S. Beijer, P. Rider, and P. Neuner, Nucleic Acids Res., 1987, 15, 4837; R. Zuckerman, D. Corey, and P. Shultz, Nucleic Acids Res., 1987, 15, 5305; P. Li, et al., Nucleic Acids Res., 1987, 15, 5275.); 5′-thiol-modifier C6 S—S and thiol group at the 3′-terminus (see e.g. B. A. Connolly and R. Rider, Nucleic Acids Res., 1985, 13, 4485; B. A. Connolly, Nucleic Acids Res., 1987, 15, 3131-3139; N. D. Sinha and R. M. Cook, Nucleic Acids Res., 1988, 16, 2659; A. Kumar, S. Advani, H. Dawar, and G. P. Talwar, Nucleic Acids Res., 1991, 19, 4561; R. Zuckermann, D. Corey, and P. Schultz, Nucleic Acids Res., 1987, 15, 5305; K. C. Gupta, P. Sharma, S. Sathyanarayana, and P. Kumar, Tetrahedron Lett., 1990, 31, 2471-2474; U. Asseiine, E. Bonfils, R. Kurfurst, M. Chassignol, V. Roig, and N. T. Thuong, Tetrahedron, 1992, 48, 1233-1254; Gregg Morin, Geron Corporation, Personal Communication.); spacer C3, spacer C12, and dSpacer phosphoramidites (see e.g. M. Durard, K. Chevrie, M. Chassignol, N. T. Thuong, and J. C. Maurizot, Nucleic Acids Res., 1990, 18, 6353; M. Salunkhe, T. F. Wu, and R. L. Letsinger, J. Amer. Chem. Soc., 1992, 114, 8768-8772; N. G. Dolinnaya, M. Blumenfeld, I. N. Merenkova, T. S. Oretskaya, N. F. Krynetskaya, M. G, Ivanovskaya, M. Vasseur, and Z. A. Shabarova, Nucleic Acids Res., 1993, 21, 5403-5407; M. Takeshita, C. N. Chang, F. Johnson, S. Will, and A. P. Grollman, J. Biol. Chem., 1987, 262, 10171-10179; M. W. Kalnik, C. N. Chang, A. P. Grollman, and D. J. Patel, Biochemistry, 1988, 27, 924-931.); 3′-amino-modifier C7 CPG (see e.g. J. G. Zendegul, K. M. Vasquez, J. H. Tinsley, D. J. Kessler, and M. E. Hogan, Nucleic Acids Res., 1992, 20, 307.); 3′-amino photolabile CO CPG (see e.g. D. J. Yoo and M. M. Greenberg, J. Org. Chem., 1995, 60, 3358-3364.; H. Venkatesan and M. M. Greenberg, J. Org. Chem., 1996, 61, 525-529; D. L. McMinn and M. M. Greenberg, Tetrahedron, 1996, 52, 3827-3840.

The component modulating serum half-life is preferably an albumin or polyethylene glycol (PEG).

The pharmaceutically active compound or diagnostically active compound is preferably selected from the group consisting of (a) a fluorescent dye, (b) a photosensitizer, (c) a radionuclide, (d) a contrast agent for medical imaging, (e) a cytokine (f) a toxic compound, (g) a chemokine, (h) a pro-coagulant factor, (i) an enzyme for pro-drug activation, or (k) an ACE inhibitor, a renin inhibitor, an ADH inhibitor, an aldosteron inhibitor, or an angiotensin receptor blocker.

The fluorescent dye is preferably a component selected from Alexa Fluor or Cy dyes.

The photosensitizer is preferably phototoxic red fluorescent protein KillerRed or haematoporphyrin.

The radionuclide is preferably either selected from the group of gamma-emitting isotopes, more preferably ^{99m}Tc, ¹²³I, ¹¹¹In, and/or from the group of positron emitters, more preferably ¹⁸F, ⁸⁴Cu, ⁸⁸Y, ¹²⁴I, and/or from the group of beta-emitter, more preferably ¹³¹I, ⁹⁰Y, ¹⁷⁷Lu, ⁶⁷Cu or from the group of alpha-emitter, preferably ²¹³Bi, ²¹¹At.

A contrast agent as used herein is a substance used to enhance the contrast of structures or fluids within the body in medical imaging. Common contrast agents work based on X-ray attenuation and magnetic resonance signal enhancement.

The cytokine is preferably selected from the group consisting of IL-2, IL-12, TNF-alpha, IFN alpha, IFN beta, IFN gamma, IL-10, IL-15, IL-24, GM-CSF, IL-3, IL-4, IL-5, IL-6, IL-7, IL-9, IL-11, IL-13, LIF, 0080, B70, TNF beta, LT-beta, CD-40 ligand, Fas-ligand, TGF-beta, IL-1alpha and IL-1 beta.

The toxic compound is preferably a small organic compound or a polypeptide, more preferably a toxic compound selected from the group consisting of calicheamicin, maytansinoid, neocarzinostatin, esperamicin, dynemicin, kedarcidin, maduropeptin, doxorubicin, daunorubicin, auristatin; Ricin-A chain, modeccin, truncated Pseudomonas exotoxin A, diphtheria toxin and recombinant gelonin.

The chemokine is preferably selected from the group consisting of IL-8, GRO alpha, GRO beta, GRO gamma, ENA-78, LDGF-PBP, GCP-2, PF4, Mig, IP-10, SDF-1alpha/beta, BUNZO/STRC33, I-TAC, BLC/BCA-1, MP-1 alpha, MIP-1 beta, MDC, TECK, TARC, RANTES, HCC-1, HCC-4, DC-CK1, MIP-3 alpha, MIP-3 beta, MCP-1-5, eotaxin, Eotaxin-2, 1-309, MPIF-1, 6Ckine, CTACK, MEC, lymphotactin and fractalkine.

The pro-coagulant factor is preferably a tissue factor.

The enzyme for pro-drug activation is preferably an enzyme selected from the group consisting of carboxy-peptidases, glucuronidases and glucosidases.

The present invention relates in a third aspect to an ex vivo method of inhibiting the enzymatic activity of FXIIa comprising contacting the inhibitor, fusion protein or fusion construct of the invention with FXIIa, wherein FXIIa is preferably present in a blood, plasma or serum sample.

The method may in principle likewise be performed in vivo. As discussed herein above, inhibitors of FXIIa are currently used in coagulation test used in research labs and increasingly in the clinic. The above ex vivo method may be employed in such coagulation tests. The blood; plasma or serum sample is preferably a human blood, plasma or serum sample. In research labs also samples other than blood, plasma or serum may be of interest; e.g. to test the performance of recombinant form of FXIIa.

The present invention relates in a fourth aspect to a pharmaceutical composition comprising the inhibitor, fusion protein or fusion construct of the invention.

In accordance with the present invention, the term “pharmaceutical composition” relates to a composition for administration to a patient, preferably a human patient. The pharmaceutical composition of the invention comprises at least one inhibitor of the invention. It may, optionally, comprise further molecules capable of altering the characteristics of the compounds of the invention thereby, for example, stabilizing, modulating and/or activating their function. The composition may be in solid, liquid or gaseous form and may be, inter aria, in the form of (a) powder(s), (a) tablet(s), (a) solution(s) or (an) aerosol(s).

These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skits and judgement of the ordinary clinician or physician. Generally, the regimen as a regular administration of the pharmaceutical composition should be in the range of 1 μg to 5 g units per day. However, a more preferred dosage might be in the range of 0.01 mg to 100 mg, even more preferably 0.01 mg to 50 mg and most preferably 0.01 mg to 10 rug per day.

Furthermore, the total pharmaceutically effective amount of pharmaceutical composition administered will typically be less than about 75 mg per kg of body weight, such as for example less than about 70, 60, 50, 40, 30, 20, 10, 5, 2, 1, 0.5, 0.1, 0.05, 0,01, 0.005, 0,001, or 0.0005 mg per kg of body weight. More preferably, the pharmaceutically effective amount of pharmaceutical composition will be less than 2000 nmol of the inhibitor per kg of body weight, such as for example less than 1500, 750, 300, 150, 75, 15, 7.5, 1.5, 0.75, 0.15, 0.075, 0,015, 0.0075, 0.0015, 0.00075 or 0.00015 nmol of the inhibitor per kg of body weight.

The length of treatment needed to observe changes and the interval following treatment for responses to occur vary depending on the desired effect. The particular amounts may be determined by conventional tests which are well known to the person skilled in the art.

Pharmaceutical compositions of the invention preferably comprise a pharmaceutically acceptable carrier or excipient. By “pharmaceutically acceptable carrier or excipient” is meant a non-toxic solid, semisolid or liquid filler, diluent, encapsulating material or formulation auxiliary of any type (see also Handbook of Pharmaceutical Excipients 6ed. 2010, Published by the Pharmaceutical Press), Examples of suitable pharmaceutical carriers and excipients are well known in the art and include phosphate buffered saline solutions, water, emulsions, such as oil/water emulsions, various types of wetting agents, sterile solutions, organic solvents including DMSO etc. Compositions comprising such carriers or excipients can be formulated by well known conventional methods. The pharmaceutical composition may be administered, for example, orally, parenterally, such as subcutaneously, intravenously, intramuscularly, intraperitoneally, intrathecally, transdermally, transmucosally, subdurally, locally or tepically via iontopheresis, sublingually, by inhalation spray, aerosol or rectally and the like in dosage unit formulations optionally comprising conventional pharmaceutically acceptable carriers or excipients.

The present invention relates in a fifth aspect to the inhibitor, fusion protein or fusion construct of the invention for use in the treatment or prevention of a thrombotic disease, preferably a thrombotic disease selected from thrombus, deep vein thrombosis, hereditary angioedema and diabetic macular edema.

Specific inhibition of FXII has proven to be a safe anti-thrombotic strategy in various preclinical models of thrombus formation, including primates. The inhibitors of the invention, in particular FXII618 are therefore suitable for the treatment or prevention of a thrombotic disease. Non-limiting examples of a thrombotic disease are thrombus, deep vein thrombosis, hereditary angioedema and diabetic macular edema, FXII618 is not only itself a therapeutic compound but may also serve as a lead compound for the development of further therapeutic compounds. FXII618 is a direct inhibitor of FXIIa and therefore has the distinct advantage over previously developed inhibitors to interfere both with the thrombotic and the inflammatory functions of coagulation FXII. The inhibitors of the invention could be particularly useful in acute situations like extracorporeal circulation during heart surgery or prevention of secondary stroke events.

Another object of the present invention is to provide a method of treatment and/or prevention of a thrombotic disease, preferably a thrombotic disease selected from thrombus, deep vein thrombosis, hereditary angioedema and diabetic macular edema comprising administering a therapeutically effective amount of an inhibitor, fusion protein or fusion construct of the invention to a patient in need thereof.

Administering, as it applies in the present invention, refers to contact of the inhibitor of the invention with the subject to be treated, being preferably a human. A therapeutically effective amount of the inhibitor, when administered to a human or animal organism, is an amount of the inhibitor which induces the detectable pharmacologic and/or physiologic effect of inhibiting the enzymatic activity of the coagulation enzyme FXIIa.

For systemic administration, a therapeutically effective amount or dose can be estimated initially from in vitro assays. For example, a dose can be formulated in animal models to achieve a circulating concentration range that includes the 1050 as determined in cell culture. Such information can be used to more accurately determine useful doses in humans. Initial doses can also be estimated from in vivo data, e.g. animal models, using techniques that are well known in the art. One of ordinary skill in the art can readily optimize administration to humans based on animal data and will, of course; rely on the subject being treated, on the subject's weight, the severity of the disorder, the manner of administration.

The present invention relates in a fifth aspect to the use of the inhibitor, fusion protein or fusion construct of the invention for coating a vessel, tube, needle, syringe, membrane or medical device.

An anti-coagulant is a medication that helps to prevent clots from forming in blood. In view of the anti-coagulation effect of the inhibitors of the invention they are also suitable coating for vessels, tubes, needles, syringes, membranes or medical devices. Such coated objects are advantageous as compared to corresponding non-coated objects, as the coating prevents contact activation-driven thrombosis. For example, CTI has previously been used as a surface modifier with an improved blood compatibility outcome.^{27, 28} The small size and synthetic nature of FXII618 make this inhibitor particularly suited for conjugating it to blood-contacting surfaces, like catheters used in coronary intervention or gas-exchanging capillaries, in cardiopulmonary bypass systems. Coating may be achieved by covalently linking the inhibitor to the surface of a vessel, tube, needle, syringe, membrane or medical device.

The present invention relates in a seventh aspect to a kit for testing blood coagulation comprising the inhibitor, fusion protein or fusion construct of the invention.

As inhibitors of FXIIa are currently used in coagulation test such kits for performing such test are within the scope of the invention. The kit preferably comprises instructions how to use the kit. The various components of the kit may be packaged in one or more containers such as one or more vials. The vials may, in addition to the components, comprise preservatives or buffers for storage.

The present invention also provides for a diagnostic assay in which the inhibitor, fusion protein or fusion construct of the invention is used to inhibit factor XIIa or to bind to specifically bind to factor XIIa, e.g. in blood, plasma or serum. Non-limiting examples of diagnostic assay comprise TGA and thromboelastometry.

As described herein above, inhibitors of FXIIa are currently used in coagulation assays and consequently also the novel FXIIa inhibitors of the invention may be used in such assays. A suitable form of such an assay is a TGA and thromboelastometry comprising assay.

The figures show.

FIG. 1 Phage selection of bicyclic peptide FXIIa inhibitor. (a) Sequences of peptides isolated against β-FXIIa after 3 selection rounds. The three cysteines highlighted in grey were cyclized with the thiol-reactive linker TATA prior to affinity panning. Sequence similarities between peptides are highlighted in color. For Kis, average values of at least two measurements are indicated. (b) Activity and specificity of the best two FXIIa inhibitors selected from the library. Standard deviations are indicated. (c) aPTT and PT of a previously developed bicyclic peptide FXIIa inhibitor (FXII402) and the two newly developed inhibitors FXII512 and FXII516. Standard deviations are indicated.

FIG. 2 Affinity maturation, specificity profiling and inhibitory activity of bicyclic peptide FXIIa inhibitors. (a) Sequences of peptides isolated from a semi-randomized peptide library based on FXII516. Sequence similarities between peptides are highlighted in color. (b) Improving binding affinity of FXII516 by three rounds of amino acid substitutions. Indicated standard deviations are calculated based on three Ki values. (c) Target selectivity of FXII618. Standard deviations are calculated based on three or more Ki values. (d) Coagulation parameters aPTT and PT, and FXII activity (FXII:c) at the indicated FXII618 concentrations. Standard deviations of aPTT, PT and FXII:c are calculated based on three measurements.

FIG. 3 Structure of CTI and bicyclic peptide FXII618. (a) Polypeptide sequences of CTI (left) and FXII618 (right). The chemical linker TATA connects the three cysteines of the bicyclic peptide via thioether bonds. Residues binding to the specificity pockets S2, S1 and S1′ of FXIIa are indicated. (b) Structure models for CTI (upper figure) and FXII618 (lower) bound to FXIIa. The TATA linker of the bicyclic peptide is shown in green. (c) Superposition of the combining loops of CTI and FXII618 bound to FXIIa. (d) Dihedral angles of phi (solid lines) and psi (dashed lines) of combining loops in CTI bound to FXIIa (orange), free CTI (red) and FXIIa-bound FXII618 (blue). (e, f) Combining loops of CTI (e) and FXII618 (f) illustrating the complementarity of the inhibitors to the FXIIa active site region.

FIG. 4 Comparison of FXII618 and CTI. (a) Specificity profile and inhibitory activity of CTI (upper panel) and FXII618 (lower panel). The residual activity of several trypsin-like serine proteases is shown. Standard deviations are shown. (b) Comparison of aPTT in citrated platelet poor plasma in the presence of the same quantities of FXII618 and CTI. Standard deviations are calculated based on three measurements. (c) Thrombin generation by CAT in citrated platelet poor plasma triggered by elagic acid (intrinsic pathway activation) in the presence of various CTI (upper panel) and FXII618 concentrations (lower panel).

FIG. 5 Comparison of FXII618 and CTI in low-TF induced TGA in plasma by CAT. Thrombin generation in citrated platelet poor plasma was triggered by 0, 1 and 0.25 pM TF in the absence or presence of 100 μg/mL inhibitor CTI or FXII618.

FIG. 6 Structure-activity relationship (SARI of the FXIIa inhibitor.

FIG. 7 Chemical structure of connecting molecules.

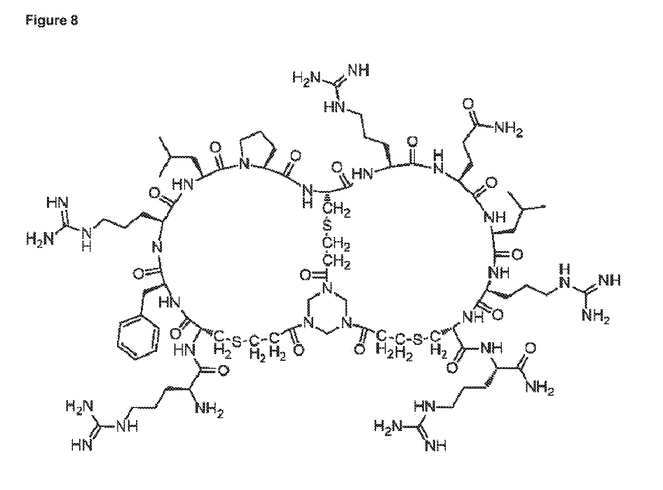

FIG. 8 Chemical ring structure of FXII618.

FIG. 9 Amino acids sequences inhibitors of the coagulation enzyme FXIIa which have been identified in connection with the present invention. The cysteins in theses amino acids sequences inhibitors are linked with TATA.

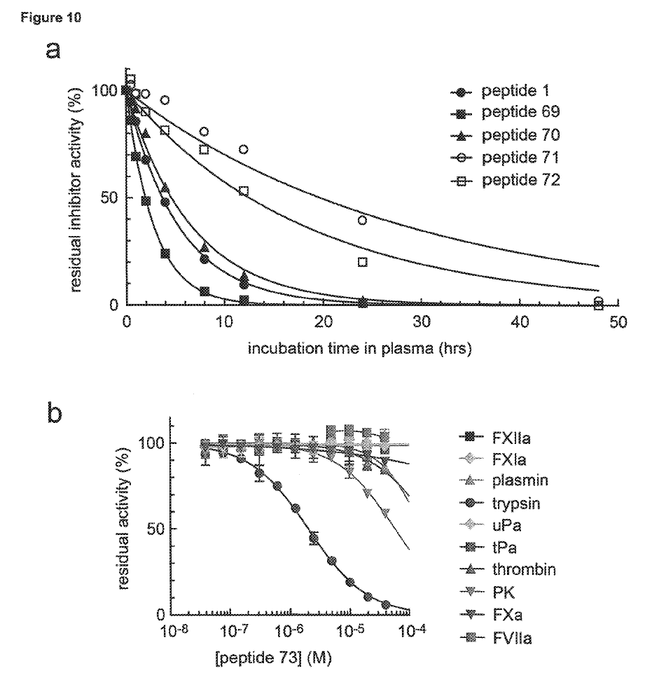

FIG. 10 (a) Stability of bicyclic peptides in human plasma. The apparent IC₅₀ was determined after incubating the peptides in human plasma at 37° C. for the indicated time periods. Average values of three measurements are indicated. (b) Specificity of bicyclic peptide 73 (FXII801). Average values of at least three measurements. Standard deviations are indicated.

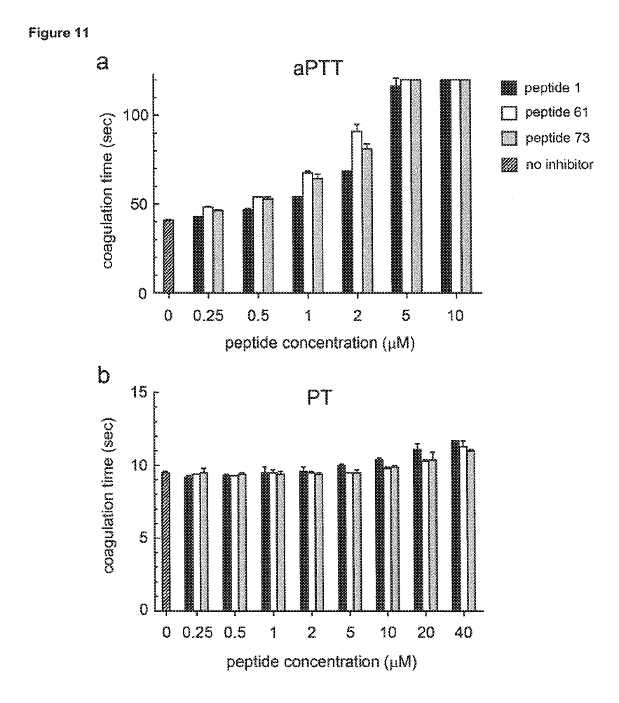

FIG. 11 Coagulation parameters aPTT (a) and PT (b) for bicyclic peptides 1 (FXII618), 61 (FXII800) and 73 (FXII801). Standard deviations of aPTT, PT are calculated based on three measurements.

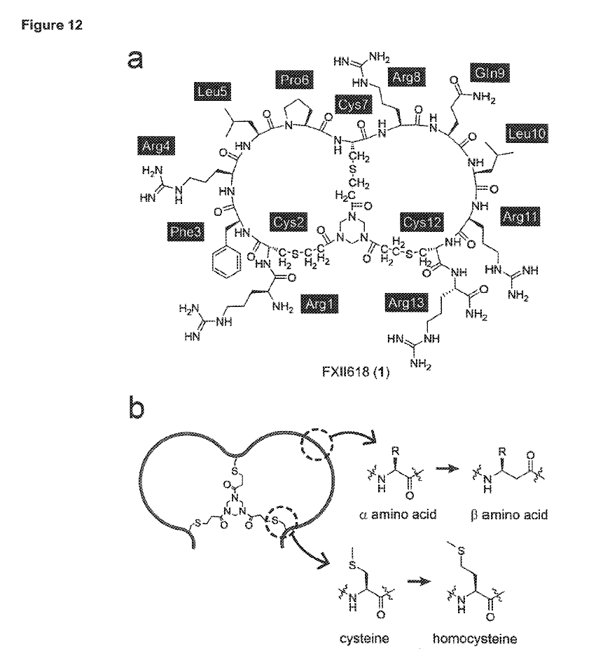

FIG. 12 (a) Chemical structure of FXII618 (1). (b) Affinity maturation strategy. Carbon atoms are inserted into the peptide backbone by replacing α-amino acids with β-amino acids, or by replacing cysteines connected to the cyclization linker with cysteine homologues having longer side chains.

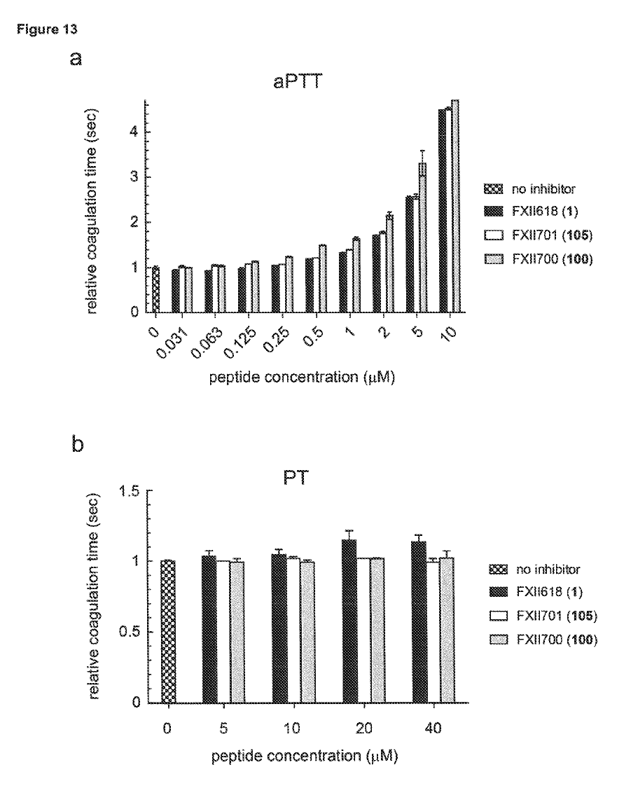

FIG. 13 Coagulation parameters aPIT (a) and PT (b) for FXII618, FXII700 and FXII701.

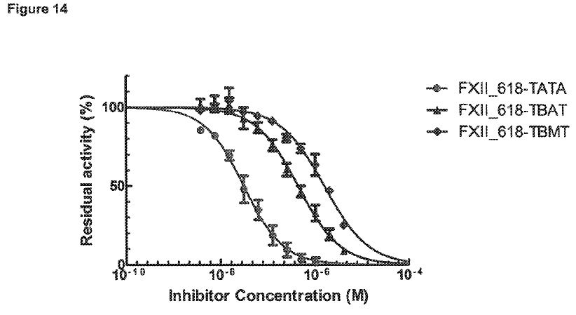

FIG. 14 Activity and specificity of FXII618-TATA, FXII618-TBAT and FXII618-TBMT. Standard deviations are indicated.

The examples illustrate the invention.

### EXAMPLE 1—GENERATION AND TESTING OF FXII618

### Example 1.1—Experimental Procedures

Phage Selections Against Activated Human FXII

The bicyclic peptide phage display libraries (library 3×3, 4×4 and 6×6) were produced and three rounds of phage selection were performed following previously described procedures.29 For each selection, phage were produced in 0.5 L of bacterial culture. Phage were purified by polyethylene glycol (PEG) precipitation. Cysteine residues were reduced with tris(2-carboxyethyl)phosphine (TCEP, 1 mM, 42° C., 1 h) prior to chemical reaction with thiol-reactive reagents 1,3,5-triacryloyl-1,3,5-triazinane (TATA, 150 μM, 30° C., 1 h) or N,N′,N″-(benzene-1,3,5-triyl)-tris(2-bromoacetamide) (TBAB, 40 μM, 30° C., 1 h) in 80% aqueous buffer (20 mM NH₄HCO₃, 5 mM EDTA, pH 8.0) and 20% acetonitrile. Human 11-FXIIa (HFXIIAB; Molecular innovations. Novi, Mich., USA) was biotinylated and 5 μg immobilized on streptavidin or neutravidin magnetic beads. Binders were eluted at acidic pH. In affinity maturation selections, less biotinylated FXIIa was used: either 0.2 μg in both rounds of selection or 2 μg in the first round and 0.2, 0.5 or 1 μg in the second round.

Cloning of the Affinity Maturation Library

Randomized DNA sequences were appended by degenerate primers to the gene of phage p3 in a PCR reaction and the product inserted into the phage vector pECO2 at the two Sill restriction sites.²⁹ Forward primer: VBfLvb21_for (TATGCGGCCCAGCCGGCCATGGCANNKTGTNNKAGGCTGNNKTGCNNKCAGITGNNKTGTNNKG GTTCTGGCGCTG) (SEQ ID NO: 91), reverse primer sfi2notfo (CCATGGCCCCCGAGGCCGCGGCCGCATTGACAGG) (SEQ ID NO: 92). Electroporation of 4.4 μg ligated vector into TG1 cells yielded 8.8×106 colony forming units (c.f.u.) on large (140 mm diameter) chloramphenicol (30 μg/mL) 2YT agar plates Colonies were scraped off the plates with 2YT media, supplemented with 15% v/v glycerol and stored at −80° C.

aPTT, PT and FXII coagulant activity (FXII:c) measurements Coagulation times (aPTT, PT) and FXIIa activity were determined in human plasma using an automated blood coagulation analyzer (Sysmex CS-5100, Siemens, Eschborn, Germany) with according to the manufacturer's instructions (Siemens). Extrinsic coagulation was triggered with Innovin® (recombinant human tissue factor, synthetic phospholipids and calcium in stabilized HEPES buffer system; Dade Behring/Simens). Intrinsic coagulation was triggered with Pathromtin* SL (Siemens). Human plasma used in this study was derived from a pool of fresh frozen plasma units provided by the Service Regional Vaudois de Transfusion Sanguine, Switzerland.

Thrombin Generation Assays: Calibrated Automated Thrombography (CAT)

Thrombin generation was performed as previously described with some modifications.30 In brief, 60 μL platelet poor plasma (PPP), 20 μL FXIIa inhibitor/HA buffer (Hepes 20 mM, NaCl 140 mM, pH 7.4, 5 mg/mL BSA), and 20 μL PPP reagent, PPP LOW reagent, MP reagent or Actin FS (1:170 diluted in HA buffer) were mixed in a 96-well microtiter plate (Immulon 2HB; Thermo Fisher Scientific) and incubated for 15 min at 37° C. Thrombin generation was triggered by the addition of 20 μL of substrate/calcium chloride buffer (FLUKA) at 37° C. Samples were tested in triplicate for each condition. Fluorogenic thrombin substrate hydrolysis was measured every 20 s for 120 min on a microplate fluorometer (Fluoroskan Ascent FL; Thermo Fisher Scientific) with a 390/460-nm (excitation/emission) filter set. Data analysis was performed on the Thrombinoscope software (Synapse By). PPP reagent (TF 5 μM, PL 4 μM), PPP LOW reagent (TF 1 μM, PL 4 μM), MP reagent (PL 4 μM), thrombin calibrator and FLUKA were purchased from Synapse By.

Blood Collection for Thrombin Generation Measurement Post-Addition of the Inhibitor in Whole Blood

Venous blood was collected from healthy volunteers by antecubital venipuncture with 19-gauge needles in 3,2% (w/v) citrated Monovette plastic tubes. In order to minimize contact activation, the first collection tube containing EDTA was discarded according to standard procedures. FXII618 was added soon after the blood collection (1 min) to the tube to a final concentration of 100 μg/mL. Blood was then processed to PPP by an initial centrifugation step at 2,000 g for 5 min followed by a second centrifugation step at 10,000 g for 10 min. Plasma aliquots were stored at −80 until analysis.

Bicyclic Peptide Synthesis

Peptides were synthesized in house by standard solid-phase peptide synthesis using Fmoc-protected amino acids and Rink Amide AM resin. Amino acids were coupled twice at 4-fold molar excess using HBTU and HOBt as coupling reagents. All peptides synthesized have a free N-terminus and an amidated C-terminus. Peptides were cleaved from the resin under reducing conditions (90% TFA, 2.5%© H2O, 2,5% thioanisol, 2,5% phenol, 2.5% 1.2-ethanedithiol). Crude peptide at a concentration of 1 mM was reacted with either 1.5 mM TATA or TBAB in 70% aqueous buffer (20 mM NH4HCO3, 5 mM EDTA, pH 8.0) and 30% acetonitrile for 1 hour at 30° C. The cyclized peptides were purified by reversed-phase chromatography on a C18 column. Pure bicyclic peptides (>95% purify) were lyophilized and dissolved in water. Their mass was confirmed by ESI.

Protease Inhibition Assays

The inhibitory activity of bicyclic peptides was determined by incubation with proteases and quantification of their residual activity with a fluorogenic substrate. Final concentration of human b-FXIIa (HFXIIAB; Molecular Innovations) and mouse α-FXIIa (MFXIIA; Molecular Innovations) was 10 nM. Dilutions of peptides were prepared ranging from 0.2-200 mM. Fluorescence intensity was measured with an Infinite M200Pro fluorescence plate reader (excitation at 355 nm, emission at 460 nm; Tecan, Männedorf, Switzerland). The reactions were performed at RT. Sigmoidal curves were fitted to the data using the following dose response equation wherein x=peptide concentration, y=% activity of reaction without peptide, A1=100%, A2=0%, p=1. IC50 values were derived from the fitted curve.

$$y = {A}_{1} + \frac{{A}_{2} - {A}_{1}}{1 + {10}^{{( {LOG}_{x} 0 - x )}_{p}}}$$

The inhibitory constant Ki was calculated according to the equation of Cheng and Prusoff²⁹ Ki=/050/(1+([S]0/Km) wherein IC50 is the functional strength of the inhibitor, [S]0 is the total substrate concentration, and Km is the Michaelis-Menten constant.

Ex Vivo Coagulation Assays

Coagulation times (aPTT, PT) and FXIIa activity were determined, at least in duplicate, in human plasma using an automated blood coagulation analyzer (Sysmex CA-7000) with standard reagents according to the manufacturer's instructions (Siemens Healthcare, Eschborn, Germany). Human plasma used in this study was derived from a pool of fresh frozen plasma units provided by the Service Regional Vaudois de Transfusion Sanguine, Switzerland.

Homology Modeling

Homology models of β-FXIIa with tetrapeptides of CTI and FXII618 bound in the standard mechanism to the S2, S1, S1′ and S2′ sub-sites was built using X-ray cocrystal structure data sets 1PPE, 2×TT and 4AOQ as templates. Non-essential water molecules and ions were deleted in the PDB files and the peptide ligands truncated to retain only the P2, P1, P1′ and P2′ residues. β-FX is and the tetrapeptides of CTI and FXII618 were aligned with b-trypsin and the tetrapeptides of the three ligands using the alignment scripts from MODELLER software³⁰. The proteins and peptides were aligned based on conserved amino acids. b-FXIIa shares 35.7% sequence identity and 54.4% sequence similarity with b-trypsin (amino acids 16-245, chymotrypsin numbering). A distance constrain of 2.9+/−0.1 Å between the 11 amino groups of P1 arginine and the d oxygen of aspartic acid 189 in β-XIIa was defined. The MODELLER Software was used to build models with bound peptidic tetrapeptides. The model with the lowest energy was used further for molecule dynamics simulations with Amber 11 Software (R. Salomon-Ferrer, A. W. Gôtz, 2013). In the structure of FXII618, the three cysteines were manually connected with TATA. AMBER force field was used for preparing the sander input files with tleap. TIP3P waters were used to solvate the structures. The system was first minimized by holding the conformation of b-FXIIa and the bound peptide, and then only the interacting residues at S2, S1, S1′ and S2′ pocket. Finally the whole system was minimized. Then the system was heated to 300 K before an equilibration on the whole system. The production equilibration was run at 300 K with constant pressure and periodic boundary for 20 ns. The Langevin dynamics was used to control the temperature using a collision frequency of 1.0 ps-1. Then the final structures were analyzed. All the calculations were perform in the GPU-accelerated cluster ELECTRA provided by scientific IT and application support (SCITAS) at EPFL.

Inhibition of FXII Activity In Vivo

Female BALB/c mice were anesthetized with 125 mg/kg pentobarbital. After five minutes, mice were injected r.o. with either 3.5 mg/kg FXII618 combined with 3.5 mg/kg PK128 (N=3), or with PBS (N=2) as a control. Five minutes later, blood was collected from the vena cava at a 1:9 blood to citrate ratio. Plasma was prepared by centrifugation at 2,400 g 10 minutes at RT. FXIIa activity was assessed as described above.

### Example 1.2—Screening of Structurally Diverse Bicyclic Peptide Libraries

Six combinatorial phage display libraries comprising jointly more than ten billion different bicyclic peptides were developed using novel peptide cyclization reagents and more diverse peptide formats. The libraries were panned against human β-FXIIa, a naturally occurring proteolytic product of FXIIa comprising only the catalytic domain. The libraries were generated by cyclizing linear peptides of the format CXnCXnC (C=cysteine, X=random amino acid; n=3, 4, 6) displayed on phage with either of the thiol-reactive reagents 1,3,5-triacryloyl-1,3,5-triazinane (TATA) or N,N′,N″-(benzene-1,3,5-triyl)-tris(2-bromoacetamide) (TBAB). These chemical linkers impose different peptide backbone conformations in bicyclic peptides in comparison to the previously applied reagent 1,3,5-tris(bromomethyl)benzene (TBMB).^{19, 20} Affinity selections performed with TBAB-cyclized peptides led to the isolation of binders but not inhibitors. In contrast, bicyclic peptides isolated from TATA-cyclized peptide libraries yielded FXIIa inhibitors with Kᵢs for β-FXIIa ranging from 0.16+/−0.07 μM to 10.2+/−0.9 μM (FIG. 1a). Further characterization of the two most potent peptides, FXII512 (Kᵢ of 0.16+/−0.07 μM) and FXII516 (0.16+/−0.08 μM) showed that they cross-react with mouse FXIIa (β-FXIIa; Ki of 0.32+/−0.07 μM and 0.45+/−0.16 μM, respectively) (FIG. 1b). A specificity profiling with a panel of structurally and functionally related serine proteases showed that FXII512 inhibits human tissue-plasminogen activator (tPA; Kᵢ=8.8 μM), a coagulation-associated enzyme involved in fibrinolysis. FXII516 did not inhibit significantly any of the proteases tested at a concentration as high as 50 μM.

The higher selectivity of FXII516 over FXII512 in regard to FXIIa inhibition was further observed in coagulation assays. Activated partial thromboplastin time (aPTT) and prothrombin time (PT) were measured in human plasma in the presence of FXII512 or FXII516 at three different concentrations. FXII402, the previously developed inhibitor of FXIIa (cyclized with TBMB; Kᵢ=1.2 μM).¹⁸ was used for comparison. aPTT and PT measure the time to coagulation upon initiation of coagulation either via the intrinsic (aPTT) or the extrinsic pathway (PT). Selective FXIIa inhibition is expected to increase aPTT but not PT. In the case of a complete FXIIa inhibition; the aPTT and PT should be comparable to those measured in FXII-deficient plasma (aPTT>170 seconds and steady-state PT; Service and Central Laboratory of Hematology; Lausanne University Hospital, Switzerland). As shown in FIG. 1c, the newly developed bicyclic peptides were more potent than FXII402 in inhibiting the intrinsic pathway of coagulation, as observed by the longer aPTT for equal inhibitor concentrations. FXII512 inhibited the strongest the intrinsic pathway but presented a prolonged, non-physiological PT at a concentration of 50 μM and above. In contrast FXII516 efficiently inhibited the intrinsic pathway without affecting the extrinsic pathway, even at the highest inhibitor concentration tested (100 μM), and offered a promising lead. Screening the newly developed, structurally more diverse libraries had thus yielded an inhibitor that was substantially more potent than the best peptide previously developed by phage display.¹⁶

### Example 1.3—Engineering of a Potent and Selective FXIIa Inhibitor

The potency of FXII516 was further improved by altering amino acids outside the consensus region. Screening of a phage display library of the form XCXRLXCXQLXCX (consensus sequence is underlined, X=random amino acid) did not yield improved binders but the sequences showed an extended consensus sequence and gave hints for beneficial amino acid substitutions in FXII516 (FIG. 2a). Most notable is the convergent evolution of amino acids in position 1 to aliphatic amino acids and arginine, in position 3 and 6 to proline, in position 8 to alanine, and in position 11 to arginine. Variants of FXII516 with single amino acid substitutions showed up to 4-fold improved inhibitory activities (FIG. 2b; 1st round). Accumulation of further mutations by two iterative cycles of mutation and screening yielded peptide FXII618 that inhibits human R-FXIIa with a Ni of 22+/−4 nM (FIG. 2b; 2nd and 3rd round). α-FXIIa was inhibited to the same extent (Ki=19+/−1 nM). FXII618 also inhibited mouse FXIIa with a Ni of 252+/−29 nM, but not structurally-related or functionally-important proteases (FIG. 2c). FXII618 presents an 8-fold and a 60-fold improvement in inhibiting human FXIIa in comparison to its parent FXII516 and to the previously developed TBMB-cyclized inhibitor FXII402, respectively. The potency and specificity of FXII618 was further assessed in in vitro coagulation assays. At concentrations as low as 40 μM, the bicyclic peptide completely inhibited initiation of coagulation via the intrinsic pathway (aPTT>170 seconds) without affecting the extrinsic pathway (steady-state PT). Consistently, at 50 μM, FXII coagulant activity (FXII:c) was reduced to <5% (FIG. 2d).

### Example 1.4—FXII618 Mimics Binding Mode of Corn Trypsin Inhibitor

Comparison of the identified consensus sequences with binding loops of natural FXIIa inhibitors showed a striking similarity between the first ring of the most potent bicyclic peptide FXII618 (RCFRLPCRQLRCR) and the combining loop of corn trypsin inhibitor (CTI; . . . IGPRLPW . . . ) (identical amino acids are underlined). The homology was even mere pronounced for the consensus sequence found in the affinity maturation approach that led to the development of FXII618 (XCPRLPCXQL^{R}/_{K}CX; FIG. 2a) and suggested that this bicyclic peptide has the same binding mode as CTI (FIG. 3a). CTI is a 13.6 kDa protein from corn seeds that equally inhibits FXIIa and trypsin. It is broadly applied to suppress activation of contact phase in coagulation assays. The 127 amino acid protein containing 5 disulfide bridges (PBD: 1BEA) is a canonical inhibitor that obeys the so-called ‘standard mechanism’ of inhibition23 in which a peptide loop of the inhibitor binds essentially as polypeptide substrates of the enzyme. Arg34 of CTI binds into the S1 specificity pocket of FXIIa.²

It was tested if FXII618 can indeed adopt a conformation that is complementary to the active site of FXIIa by homology modeling and molecular dynamics simulation. As no co-crystal structure of CTI and FXIIa existed, also the CTI-FXIIa complex was modelled. The co-crystal structures of the homologous serine protease bovine β-trypsin and CMTI-I (a trypsin inhibitor from squash seeds; PDB: 1PPE), SGPI-1-P02 (schistocerca gregaria protease inhibitor 1; PDB: 2×TT), and SOTI-III (spinacia oleracea trypsin inhibitor PDB: 4AOQ) served as structural templates for the homology modeling. 36% of the amino acids of FXII catalytic domain (amino acids 16.254) and bovine R-trypsin are identical. CMTI-I, SGPT-1-P02 and SOTI-ill bind trypsin according to the standard mechanism. The binding loops of CTI and FXII618 formed complementary structures to FXIIa (FIG. 3b). The backbone as well as the side chain of arginine of the combining loop in CTI was superposing with the one of the first ring of FXII618 (FIG. 3c). The Ramachandran angles of the combining loop in free CTI (taken from PDB: 1BEA) remained essentially the same when the inhibitor was bound to the protease (FIG. 3d). The dihedral angles of the 4 amino acids in ring 1 of FXII618 (FRLP) were similar to those of the equivalent amino acids in the model of bound CTI (FIG. 3d). A structure representation of the enzyme-inhibitor complex underscores the complementarity of the bicyclic peptide ring 1 with the protease's active site (FIG. 30. The second ring of FXII618 (ring 2) was also fitting to the surface of FXIIa without creating any steric clashes. However, the conformation of ring 2 in the model is less certain as no specific contacts of this peptide region with the surface of FXIIa are found.

### Example 1.5—FXII618 is Superior to CTI in Suppressing Intrinsic Coagulation Initiation

The activity and target specificity of FX/1618 and CTI were compared side-by-side in inhibition assays (FIG. 4). FXIIa inhibition was comparable for both inhibitors. In contrast, the target selectivity of FXII618 was better than that of CTI as assessed with a panel of trypsin-like serine proteases sharing structural and functional similarity with FXII. While CTI inhibited substantially the three proteases trypsin (Ki=7+/−0.5 nM), plasmin (Ki=5+/−1.6 μM) and factor XIa (Ki=12.5+/−1,4 μM) (FIG. 4, upper panel), the bicyclic peptide inhibited only trypsin (Ki=5+/−0.5 μM) (FIG. 4, lower panel). Compared to CTI, FXII618 inhibited trypsin around 100-fold weaker.

FXII618 and CTI were also compared side-by-side for their ability to inhibit the initiation of the intrinsic pathway in in vitro coagulation assays (FIG. 4b). As indicated in the introduction, the cost of goods is a major limiting factor in the application of CTI.²⁴ Researchers usually limit expenses by applying the minimal amount of CTI required to largely block FXIIa (30-100 μg/mL). Therefore in this experiment the activity per mass of the two inhibitors was compared. The activity per molecule can be derived from the presented data by taking into account the different molecular weights of the two inhibitors (13.6 kDa [CFI]; 1956 Da [FXII618]=factor 7). In a first set of experiments, aPTT was measured in the presence of various inhibitor concentrations. FXII618 blocked contact-triggered coagulation completely at 100 μg/mL (aPTT>170 seconds). The same quantity of CTI delayed coagulation to a final aPTT value of only 65.4+/−0.5 seconds. At higher CTI concentrations, aPTT values appeared to converge to a plateau being far from complete inhibition. In a second set of experiments, the inhibition of contact activation by the two inhibitors was measured in a real-time thrombin generation assay (TGA)²⁵ triggered by ellagic acid (EA), using CAT (FIG. 4c). FXII618 reduced the EA-induced thrombin generation in a dose-dependent manner, reaching a complete inhibition of thrombin formation already at 20 μg/mL FXII618 (>90% reduction of endogenous thrombin potential (ETP) and peak) (FIG. 4c, lower panel). Comparable TGA results were obtained with FXII-deficient plasma. Around 5-times more CTI was needed (100 μg/mL) to reduce thrombin generation to background levels (˜90% reduction of ETP and >96% of peak; FIG. 4c, upper panel). These results show the increased potency of FXII618 over CTI in preventing activation of the intrinsic pathway. To assess the specificity of FXII618 for the intrinsic pathway, thrombin generation was induced via the extrinsic pathway using high concentration of TF (5 μM). Even at the highest concentration of 100 μg/mL FXII618, the peptide inhibitor did not affect TF-induced thrombin generation. Since contact activation can occur during venipuncture, the effect of adding FXII618 to whole blood directly after collection was tested. Similar results were obtained. Taken together, these data clearly demonstrate the strong potency and specificity of FXII618, which efficiently inhibits the contact system and thereby the initiation of the intrinsic pathway without having any effect on the extrinsic pathway.

### Example 1.6—FXII618 Efficiently Blocks Contact Activation in Low-TF Diagnostic Tests

As described in the introduction, coagulation assays triggered with low TF concentration (<1 pM) mimic best the coagulation processes under physiologic conditions. However, at the low TF trigger concentrations, contact activation contributes substantially to thrombin generation and falsifies the result. FXII618 and CTI were compared for their ability to inhibit contact activation in low-1F thrombin generation assays in plasma (FIG. 5). When thrombin generation was triggered using 1 pM TF, no significant effect of FXII618 or CTI could be observed (FIG. 5, top panel). Instead, at 0.25 pM TF, FXII618 reduced the endogenous thrombin potential to a similar extent as CTI, but delayed the lag time more potently (lag time: 12.3±0.3 and 8.0±0.2, respectively) (FIG. 5, middle panel). In thrombin generation performed in the absence of an extrinsic- or intrinsic-specific trigger, FXII618 inhibited contact activation significantly stronger than CTI (lag time: 55.8±3.7 and 38.7±1.9, and peak: 60.3±0.8 and 78.0±6.8, respectively; FIG. 5, bottom panel).

It was finally tested if the bicyclic peptide could delay even more the thrombin generation if it is added to whole blood immediately after blood sampling. Thrombin generation was tested in plasma. Similar results were found as in the experiments in which the inhibitor was added to the plasma. In the absence of an extrinsic- or intrinsic-specific trigger, FXII618 could even completely block thrombin activation. This latter finding indicates that FXII activation taking place in the blood sampling tube can be suppressed if the inhibitor is added immediately after blood collection. The improved blockade of contact activation by FXII618 in comparison to CTI unravels the potential of this synthetic inhibitor to replace CTI in low-TF diagnostic tests.

### Example 1.7—Selective Inhibition of Intrinsic Coagulation in Mice

FXII618 is cross-reactive with mouse FXIIa which allowed testing its activity in mice. As the Ki for mouse FXIIa (252+/−29 nM) is 11-times higher than for human FXIIa (22+/−4 nM), FXII618 was combined with a previously described bicyclic peptide inhibitor of plasma kallikrein (PK), PK12819. Upon activation of small amounts of FXII with negatively charged surfaces, FXIIa activates plasma kallikrein (PK) which reciprocally activates larger amounts of FXII. Inhibiting both proteases results in synergic blockade of FXIIa by inhibiting the activity of FXII as well as its activation. Administration of 3.5 mg/kg FXII618 combined with 3.5 mg/kg PK128 reduced FXII activity to 49.7+/−13.9%. Consistent with the ex vivo data obtained in human plasma, PT values were comparable between inhibitor-treated (7.1+/−0.2 seconds) and PBS-control mice (6.7+/−0.3 seconds), showing that the extrinsic pathway remained unaffected.

### EXAMPLE 2—NATURAL AND UNNATURAL AMINO ACID SUBSTITUTIONS IN FXII618

### Example 2.1—Material and Methods

Bicyclic Peptide Synthesis:

Peptides were synthesized in house by standard solid-phase peptide synthesis using Fmoc-protected amino acids and Rink Amide AM resin. Amino acids were coupled twice at 4-fold molar excess using HBTU and HOBt as coupling reagents. Coupling of unnatural amino acids was performed manually by adding 2 eq of Fmoc-protected amino acid, 2 eq of HATU and 4 eq of DIPEA. All peptides synthesized have a free N-terminus and an amidated C-terminus. Peptides were cleaved from the resin under reducing conditions (90% TFA, 2.5% H₂O, 2,5% thioanisol, 2,5% phenol, 2,5% 1.2-ethanedithiol). Crude peptide at a concentration of 1 mM was reacted with 1,5 mM TATA in 70% aqueous buffer (50 mM NH₄HCO₃, pH 8.0) and 30% acetonitrile for 1 hour at 30° C. The cyclized peptides were purified by reversed-phase chromatography on a C18 column. Pure bicyclic peptides (>95% purity confirmed by analytical HPLC) were lyophilized and dissolved in water. Their mass was confirmed by ESI.

Protease Inhibition Assays

The inhibitory activity of bicyclic peptides was determined by incubation with proteases and quantification of their residual activity with a fluorogenic substrate. Residual enzymatic activities were measured in buffer containing 10 mM Tris-CI, pH 7.4, 150 mM NaCl, 10 mM MgCl₂, 1 mM CaCl₂, 0.1% w/v BSA, 0.01% v/v Triton-X100, and 5% v/v DMSO in a volume of 150 μL. In a first series of experiments 50 μM of the fluorogenic substrate Z-Gly-Gly-Arg-AMC and 2.5 nM of enzyme (human p-FXIIa, HFXIIAB; Molecular Innovations) was used. Later, the more sensitive substrate Boc-Gin-Gly-Arg-AMC was used allowing to reduce the enzyme concentration to 0.5 nM and hence be able to measure lower Kᵢ values. Control experiments showed that very similar inhibitory constants of the FXIIa-inhibitors were obtained when comparing the two different fluorogenic substrates using the corresponding Kₑ values mentioned below.

Dilutions of peptides were prepared ranging from 0.00001-4 μM. Fluorescence intensity was measured with an Infinite M200Pro fluorescence plate reader (excitation at 368 nm, emission at 467 nm; Tecan, Männedorf, Switzerland). The reactions were performed at 25° C., Sigmoidal curves were fitted to the data using the following dose response equation wherein x=peptide concentration, y=% activity of reaction without peptide, A₁=100%, A₂=0%, p=1. IC₅₀ values were derived from the fitted curve.

$$y = {A}_{1} + \frac{{A}_{2} - {A}_{1}}{1 + {( {10}^{( {LOG}_{x} 0 - x )} )}_{p}}$$

The inhibitory constant Kᵢ was calculated according to the equation of Cheng and Prusoff Kᵢ=IC₅₀/(1+([S]₀/Kₘ) wherein IC₅₀ is the functional strength of the inhibitor, [S]₀ is the total substrate concentration, and Kₘ is the Michaelis-Menten constant. Kₘ for the two substrates Z-Gly-Gly-Arg-AMC and Boc-Gln-Gly-Arg-AMC have been determined to be 180±31 and 256±42 μM, respectively (mean±SD, n=4).

For the specificity testing the following final concentrations of human serine proteases were used: tPA (Molecular Innovations) 7.5 nM, uPA (Molecular Innovations) 1.5 nM, factor XIa (Innovative Research, Novi, Mich., U.S.) 6 nM, PK (Innovative Research) 0.25 nM, thrombin (Molecular Innovations) 1 nM, plasmin (Molecular Innovations) 2.5 nM, trypsin (Molecular Innovations) 0.05 nM, factor VIIa (Haematologic Technologies Inc.) 50 nM and factor Xa (Haematologic Technologies Inc,) 6 nM.

Dilutions of peptides were prepared ranging from 0.04 to 40 μM. For the determination of the IC₅₀ inhibitory constants, the following fluorogenic substrates were used at a final concentration of 50 μM: Z-Phe-Arg-AMC (for PK; Bachem, Bubendorf, Switzerland), Boc-Phe-Ser-Arg-AMC (for factor XIa; Bachem); Z-Gly-Gly-Arg-AMC (tPA, uPA, thrombin, and trypsin; Bachem), H-D-Val-Leu-Lys-AMC (for plasmin; Bachem) and D-Phe-Pro-Arg-ANSNH-C₄H, (for FVIIa and FXa; Haematologic Technologies Inc.).

Plasma Stability Assays

Peptide (2 μl of 2 mM in H₂O) was added to 398 μl human plasma (final peptide concentration was 10 μM in 400 μl final volume). The mixture was incubated in the water bath at 37° C. At 8 different time points (0 h, 30 min, 1 h, 2 h, 4 h, 8 h, 12 h, 24 h and in some cases 48 h) a sample of 30 μl was removed, diluted to 200 μl with the same buffer used for protease inhibition assay (see above) and heat-inactivated for 20 min at 65° C. During this step proteases present in plasma are being inactivated, that would otherwise interfere with the subsequently performed enzymatic assay. Control experiments showed that incubated peptides are completely resistant to the heat-inactivation procedure and inactivated plasma without peptide inhibitors only weakly affected the activity of βFXIIa in the enzymatic assay. After heat-inactivation of the plasma samples, the latter were kept at −20° C. until their inhibitory potencies against FXIIa were analyzed in a protease inhibition assay. Before analysis, plasma samples were centrifuged for 5 min at 16′000 g and the supernatant was collected. A twofold dilution series ranging from 0.0005-0.5 μM was prepared. IC₅₀ values were derived from the fitted curve using the equation indicated above. Residual inhibition in % was calculated using the following formula: IC_{50, 0h}/IC_{50, xh}*100 wherein IC_{50, 0h} is the functional strength of the inhibitor at time point 0 and IC_{50, xh} the functional strength of inhibitor after one of the different plasma incubation period mentioned above.

aPTT and PT Coagulant Activity Measurements

Coagulation times (aPTT, PT) were determined in human plasma using a blood coagulation analyzer (STart. Hemostasis coagulation analyzer, Diagnostica Stago). Extrinsic coagulation was triggered with Innovin (recombinant human tissue factor, synthetic phospholipids and calcium in stabilized HEPES buffer system; Dade Behring; Siemens). Intrinsic coagulation was triggered with Pathromtin* SL (Siemens). Pooled normal human plasma used in this study was provided by Innovative research (USA).

### Example 2.2—Results and Discussion

In Example 2.2 reference is made peptides by numbers 1 to 73. The peptide of number I is FXII618. Structural features and inhibitory capacity of the peptide of numbers 3 to 72 are also shown in FIG. 6. The structural features and inhibitory capacity of peptide number 73 are detailed in the example.

Improving the Inhibition Activity

Based on sequence similarities of peptides isolated in the phage selections against FXIIa, the four amino acids of the first ring (Phe3, Arg4, Leu5, Pro6) and the middle two amino acids of the second ring of peptide 1 (Gin9, Leu10) appeared to be most important for the binding (FIG. 1). In a first attempt to improve the inhibitory activity of peptide 1, it was chosen to replace the amino acids in these positions with natural amino acids having similar side chains. Suitable amino acid substitutions were chosen using the two scoring matrices BLOSUM62 and PAM250 that indicate the evolutionary substitution probability of an amino acid by another one (FIG. 6). To allow precise quantification of the synthesized peptides by absorption spectrometry, a tryptophan residue was appended to the C-terminus of all peptides. This peptide 2 had a Kᵢ of 26±8 nM and thus an activity comparable to peptide 1. A total of 26 bicyclic peptides, each having one amino acid substituted in one of the six positions, were synthesized. None of the peptides had an improved Kᵢ but valuable structure-activity relationship data was obtained. In the position of Phe3, replacement of phenylalanine with tryptophan conserved the activity (peptide 3; K; 26±2). All other amino acid substitutions in this position reduced the activity more than 2-fold (tyrosine, leucine, alanine; peptides 4-6) or more than 10-fold (valine, isoleucine, proline; peptides 7-9). In position Arg4, only the substitution to lysine was tested as this residue should have a positive charge to form ionic interaction with an aspartate at the bottom of the S1 binding pocket of FXII. Lysine in this position reduced the inhibitory activity 10-fold (peptide 10; Kᵢ=283±21). In the position of Leu5, all substitutions, even those with amino acids closely resembling leucine such as valine (peptide 11) or isoleucine (peptide 12), reduced the affinity more than 50-fold. Peptides containing methionine (peptide 13) or alanine (peptide 14) in this position did not inhibit FXIIa at all at the highest concentration tested (3 μM). In position Pro6, two of the amino acid replacements reduced the activity less than 2-fold, namely alanine (peptide 15) and leucine (peptide 18). In the second macrocyclic ring, all substitutions reduced the activity of peptide 2 substantially. In the position of Gln9 the smallest loss was found for aspartate (peptide 21; 54-fold) and in position Leu10 for isoleucine (peptide 26; 8-fold).

In a second attempt to improve the activity of peptide 1, it was chosen to replace the amino acids with unnatural residues that structurally resemble the original ones more closely (FIG. 6). In position Phe3, unnatural amino acids were tested that resemble phenylalanine (peptides 29-32) and tryptophan (peptides 33-37), as peptides with these two natural amino acids were most active (peptides 2 and 3). Two of the phenylalanine analogues reduced the activity around 30-fold (peptides 29 and 30) and two did not inhibit FXIIa at the highest concentration tested (1 μM; peptides 31 and 32). One out of the five peptides with tryptophan analogues had slightly improved activity: peptide 33 containing 2-naphtylalanine had a 2-fold improved inhibitory activity (K; =12±4). In position Arg4, the amino acid was replaced with one lysine analogue (homolysine; peptide 38) and three arginine analogues (homoarginine, norarginine and 4-guanidinophenylalanine; peptides 39-41). All these substitutions reduced the binding affinity of the peptides 5-fold or more. In the two positions Leu5 and Leu9, several aliphatic amino acids structurally resembling leucine were tested. In position Leu5, these substitutions reduced the binding affinity of the peptides by large factors wherein the smallest drop in affinity was found for norvaline (peptide 42). In position Leu10, all the five unnatural amino acids reduced the affinity by less than 8-fold.

Many of the 48 peptide variants described above had substantially reduced activities, despite the fact that only one amino acid was replaced at a time and that most of the inserted amino acids were structurally similar to the substituted ones. Peptide 1 most likely had evolved a shape that is complementary to the surface of FXIIa and does not tolerate larger structural changes in most of the amino acid positions. A high structural shape complementarity between ligand and target was previously found for all bicyclic peptide that were studies by X-ray crystallography. In a third attempt to improve the inhibitory activity of peptide 1, it was aimed at testing amino acid substitutions that result in even smaller structural changes in the side chains. It was chosen to modify position Phe3 as this was the only site in which two substitutions with unnatural amino acids had yielded slightly improved inhibitors (FIG. 6).

Addition of a methyl group to the phenyl ring of Phe3 in ortho- (peptide 51) and para-position (peptide 53) decreased the activity around 5-fold. In contrast, a methyl group in meta-position increased the affinity 2.5-fold (peptide 52; Kᵢ=10.4±0.6). These results indicated that some space around the side chain of phenylalanine is available but that this space is limited. It was subsequently turned to even smaller structural changes in which existing atoms were replaced. Replacement of a carbon atom with nitrogen in the phenyl ring in meta-position improved the activity around 3-fold (peptide 54; Kᵢ=8.4±0.3). The same substitution in pare-position reduced the inhibitory activity around 6-fold (peptide 55). Substitution of hydrogen atoms on the phenyl ring with halogen atoms altered the activity dramatically in both directions. Fluorine in meta-position reduced the activity around 2.5-fold (peptide 57). In contrast, fluorine in ortho- and para-position enhanced the activity 2- and 11-fold, respectively (peptide 56: Kᵢ 13.1±1.2; peptide 58: Kᵢ=2.31±0.36). The large improvement resulting from fluorine in para position is most likely resulting from a change in the electron distribution. Structural modeling of the interaction of peptide 1 and FXIIa suggested that phenylalanine is buried in an aromatic pocket and is interacting with Tyr439 and His393. Presence of electron-withdrawing groups at the phenyl ring of the peptide such as fluorine likely increase the strength of the pi-pi stacking interactions with the tyrosine residue of the protease and therefore lead to higher binding affinity. It was subsequently tested more substituents in pare position that affect the electron distribution in the phenyl ring. Addition of iodine reduced the activity more than 10-fold (peptide 59) and a nitro group improved the binding affinity 6-fold (peptide 60; =4.5±0.7). Finally a variant of peptide 58 was synthesized lacking the C-terminal tryptophan that was only introduced due to its large extinction coefficient at 280 nm allowing precise quantification of the concentration based on absorption. The resulting peptide 61 differing from peptide 1 only by the para-fluoro atom on phenylalanine had a Kᵢ of 2.01±0.07 nM. The bicyclic peptide 61 also termed FXII800 is the first synthetic FXIIa inhibitor with single-digit nanomolar inhibitory constant.

Improving the Proteolytic Stability

The bicyclic peptide 1 was found to be inactivated by proteolysis when incubated in human plasma at 37° C. for extended time periods (t_{1/2}=3.9±1.9 h). While a half-life of four hours is sufficient to block contact activation in plasma samples used in in vitro applications, as demonstrated in our previous work, it may not be long enough for clinical applications in which FXIIa needs to be inhibited over longer time periods. Mass spectrometric analysis of peptide 1 incubated in human plasma showed that the first proteolytic modification is removal of a 156 Da fragment corresponding to the mass of arginine. Bicyclic peptides without the N- and C-terminal arginine had Kᵢs of 932±80 nM (peptide 62) and 28.8±2.0 nM (peptide 63), respectively. This result suggested that plasma proteases clip off the arginine at the N-terminus. Towards the improvement of the stability, amino acid substitutions in position Arg1 were searched that i) do not reduce much the binding affinity, and ii) prevent proteolytic cleavage of the first amino acid (FIG. 6). Bicyclic peptides with Arg1 replaced were synthesized without the C-terminal tryptophan residue. Natural amino acids such as isoleucine, valine and alanine in place of Arg1 all reduced the affinity >5-fold (peptides 64-66). Replacement with lysine conserved the binding affinity (peptide 67; 27.4±3.8), suggesting that a positive charge in the side chain is important, D-arginine reduced the binding affinity 3-fold (peptide 68; =89.4±2.3). Based on this structure-activity data, a number of unnatural amino acids having positively charged side chains were tested (peptides 69-72). Peptides with these amino acids had either a slightly worse or better affinity, the Kᵢs being between 18.5 and 43.1 nM.

Next the stability of the bicyclic peptides 69-72 was tested by incubating them at a concentration of 10 μM in human plasma at 37° C. for different time periods, and measuring the residual inhibitory activity. The activity of the plasma proteases was heat-inactivated by incubation of the samples at 65° C. for 10 minutes. The heat-treatment did not affect the activity of the bicyclic peptides. Two of the four peptides had an improved stability compared to peptide 1 (FIG. 10a). The longest half-life was found for peptide 71 having a norarginine residue at the N-terminus. It had a half-life of 21.7±2.5 h and thus a 5.5-fold improved stability compared to peptide 1. Then the norarginine substitution was combined with the amino acid replacement that improved best the inhibitory activity of peptide 1 (4-fluoro-phenylalanine in position 3). The resulting peptide 73, also termed FXII801 had a Kᵢ of 3.90±0.43 nM and plasma half-life of 15.5±3.9 h.

Target Selectivity

The target selectivity of peptide 73 was assessed by measuring the inhibition of structurally related or functionally important proteases, and compared to the specificity profile of peptide 1. Eight of the ten proteases were inhibited less than 20% at the highest concentration tested (40 μM). Only two of the paralogous proteases were inhibited by peptide 73, namely trypsin (1.30±0.01 μM) and plasma kallikrein (45.1±1.5 μM). The same two proteases were also inhibited by peptide 1 at similar extents. These results thus showed that the affinity towards FXIIa could be improved while the activity towards other proteases remained essentially unchanged. The new FXIIa inhibitor peptide 73 thus exhibits a good selectivity of 300-fold over trypsin (a protease that is not present in the blood) and at least a 11,000-fold selectivity over other plasma proteases.

Inhibition of the Intrinsic Coagulation Pathway

The ability to selectively suppress the intrinsic coagulation pathway was assessed in in vitro coagulation assays. Activated partial thromboplastin time (aPTT) and prothrombin time (PT) measure the time to coagulation upon initiation of coagulation either via the intrinsic (aPTT) or the extrinsic pathway (PT). These tests have been performed in human plasma in the presence of different concentrations of FXIIa-inhibitors. Selective FXIIa inhibition is expected to increase aPTT but not PT. The coagulation tests were carried out with peptide 61 having an excellent inhibitory activity, and the slightly less active but more stable variant peptide 73, and for comparison with the previously developed FXIIa-inhibitor peptide I. The presence of peptide inhibitor prolonged aPTT in a concentration dependent manner (FIG. 11a), whereas PT remained almost unaffected at a concentration of 40 μM for all peptides tested (FIG. 11b). At a concentration of 5 μM peptide inhibitor no coagulation via the intrinsic pathway occurred within 120 s. Additionally, there was a correlation between inhibitory activity against FXIIa and extent of aPTT prolongation. Peptide 61 and 73 significantly increased aPTT prolongation when compared with peptide 1.

### EXAMPLE 3—ALTERATIONS WITHIN MACROCYCLIC RINGS OF FXII618

### Example 3.1—Material and Methods

Materials

Fmoc-L-α-amino acids, O-(benzotriazol-1-yl)-N,N,N′,N′-tetramethyluronium hexafluorophosphate (HBTU), 1-hydroxybenzotriazole hydrate (HOBt), and Rink Amide AM resin were purchased from GL Biochem (China). Fmoc-8-amino acids were purchased from Chemimpex (USA) and Polypeptide (France).

Peptide synthesis Peptides were synthesized on an Advanced ChemTech 348-Ω peptide synthesizer (Aapptec, USA) by solid phase peptide synthesis using standard Fmoc procedures (0.03 mmol scale). Rink Amide AM resin was used as solid support and DMF as solvent. Each amino acid was coupled twice (4 eq, 0.2 M in DMF) using HBTU/HOBt (4 eq, 0.45 M in DMF) and DIPEA (6 eq, 0.5M in DMF). The resin was washed four times with DMF after the coupling reaction. The N-terminal Fmoc protecting group was removed with 20% (v/v) piperidine in DMF (RT, 2×5 min, 400 rpm). The resin was washed five times with DMF after deprotection.

Peptide Cleavage from the Resin

Peptides were side chain deprotected and cleaved from the Rink Amide AM resin by incubation with 5 mL cleavage cocktail (90% v/v TFA, 2.5% v/v 1,2-ethanedithiol, 2.5% w/v phenol, 2.5% v/v thioanisole, 2.5% v/v H2O) for 2 h with shaking. The resin was removed by vacuum filtration, and the peptides were precipitated with ice-cold diethyl ether (50 mL), incubated for 30 min at −20 C, and pelleted by centrifugation for 5 min at 4000 rpm (2700 g). The diethyl ether was discarded, and the precipitate was then washed twice with diethyl ether. The remaining solvent was evaporated at RT.

Peptide Cyclization with TATA:

The crude peptide (typically 50 mg) was dissolved in 6 mL 33% MeCN and 67% H₂O (giving a concentration of 3.5 mM). To the solution 1,3,5-triacryloyl-1,3,5-triazinane (TATA) (10 mM, 1.2 eq) in MeCN was added. The reaction was started by the addition of degassed aqueous NH4HCO3 buffer (60 mM, pH 8.0) until a final peptide concentration of 1 mM. The reaction was left for 1 h at a 30° C. water bath and then lyophilized.

Peptide Purification by Reversed-Phase HPLC

Modified peptide powder was dissolved in 1 mL DMSO, 2 mL MeCN containing 0.1% TFA, and 7 mL H2O containing 0.1% TFA, and purified on a preparative C18 column (Vydac C18 TP1022 250, 22 mm, 10 mm) using a linear gradient of solvent B (MeCN 0.1% v/v TFA) over solvent A (H2O, 0.1% v/v TFA) (13 min, 15-28%, flow rate: 20 mLmin⁻¹). Peaks of the desired product were identified by ESI-MS analysis and lyophilized.

Protease Inhibition Assays

The inhibitory activity of the synthesized bicyclic peptides was determined by incubation with the protease and quantification of the residual activity at various peptide concentrations using a fluorogenic substrate. A 2 mM (by weight) stock solution in H₂O was made for each peptide. For the assay, serial dilutions were made from the peptide stock solution using aqueous buffer containing 10 mM TRIS, 150 mM NaCl, 10 mM MgCl (hexahydrate), 1 mM CaCl₂ (dihydrate), 0.01% (v/v) Triton X-100, 0.1% (w/v) BSA and pH adjusted to 7.4. The final concentration of human 3-FXIIa (HFXIIAB; Molecular Innovations) was 1 nM or 0.5 nM. Dilutions of peptides were made in the range 3000-0.1 nM, depending on affinity. The final concentration of the fluorogenic substrate Boc-Q-G-R-AMC (Bachem) was 50 μM and the final DMSO content was 5%. The fluorescence was measured using an Infinite M200 Pro plate reader (Tecan) with filters 368 nm for excitation and 467 nm for emission. The measurement was performed for 60 min with a read every minute at 25° C. IC50 and Kᵢ values were calculated using GraphPad Prism 5 software.

Coagulation Assays

Coagulation times (aPTT and PT) were determined in human plasma using a STAGO STart4 Coagulation analyzer (Diagnostica) according to the manufacturer's instructions. Extrinsic coagulation was triggered with Innovin (recombinant human tissue factor, synthetic phospholipids, and calcium in stabilized HEPES buffer system; Dade Behring/Siemens). Intrinsic coagulation was triggered with Pathromtin* SL (Siemens). Human single donor plasma used in this study was provided by Innovative research (USA).

### Example 3.2 Results and Discussion

In Example 3.2 reference is made to peptides by numbers 1 and 74 to 107. The peptide of number 1 is FXII618. Structural features and inhibitory capacity of the peptide of numbers 74 to 106 are also shown in FIG. 6. The structural features and inhibitory capacity of peptide number 107 are detailed in the example.

In this example the bicyclic peptide FXII inhibitor FXII618 has been affinity maturated (1) by altering the backbone rather than the side chains. Specifically, it was proposed to insert carbon atoms in different positions of the macrocyclic ring. It was reasoned that peptides with a ring size enlarged were not sampled in the phage display screen. It was further speculated that inserting carbon atoms in some sites of the backbone might allow a better positioning of some groups in the cyclic peptide and that this in turn could potentially improve the strength of existing molecular interactions. Inserting or deleting single carbon atoms at different positions in macrocyclic rings of peptide ligands has not been reported as a systematic approach for improving binding affinity. In several examples described in the literature, atoms were inserted or deleted at the sites where the peptides are cyclized.

A technically simple and fast strategy for inserting carbon atoms into the macrocyclic rings of peptides is by replacing the canonical α-amino acids with 6-amino acids that contain an additional carbon atom between the amino and carboxyl group (FIG. 12a). β-amino acids were widely used to improve the stability of α-helical peptides.³¹⁻³⁴ In some studies, β-amino acids had improved in addition to the stability also the binding affinity, as for example in an analogue of parathyroid hormone receptor-1 agonist³⁵ or an engineered VEGF signaling inhibitor based on the Z-domain.³⁶ Towards the affinity maturation of FXII618 (1), it was chosen to substitute α-amino acids in different positions of the two macrocyclic rings with β-amino acids.

In order to identify amino acid positions in which insertion of a carbon atom could potentially improve the binding affinity of FXII618.³⁷ two series of peptide variants were synthesized: one having individual amino acids replaced by 6-alanine, and the other one having them replaced by glycine (FIG. 6). Comparison of the peptides containing β-alanine and glycine in a specific position allowed understanding if the additional carbon atom enhances binding to FXII, independent of the amino acid side chain. Substitution of Phe3, Arg4 and Leu5 to β-alanine (74, 76, 78) inactivated the inhibitor completely, allowing no comparison with the peptides having glycine in these positions (75, 77, 79; no inhibition at 2 μM). Substitution of Pro6 to 6-alanine (80) and glycine (81) reduced the affinity around 5.5- and 22-fold, respectively. The smaller loss in binding affinity found for the β-alanine variant indicated that insertion of one carbon atom in this position enhances the binding to FXII. Most likely, the additional flexibility allowed the bicyclic peptide adopting a conformation in which one or several of the existing non-covalent interactions were strengthened. Amino acid replacement at Arg8 had the opposite effect, giving a larger affinity drop for the peptide with the 6-alanine (82, 83). In the positions Gln9 and Leu10 substitutions to β-alanine and glycine reduced affinity by around the same factor of 200 (84, 85, 86, 87). Finally, in position Arg11, the loss in affinity was small and similar for the β-alanine (88) and glycine variants (89) (5-fold). Given the small loss in affinity, there was a realistic chance that insertion of β-amino acids having arginine side chains can improve the affinity of FXII618 (1).

Based on the results of the β-alanine/glycine screen, Pro6 and Arg11 were considered as the most promising sites for insertion of 6-amino acids with suitable side chains. β-amino acids can have side chains at either the alpha (C2) carbon or the beta (C3) carbon and are denoted β²- and β³-residues, respectively. Given that these carbon atoms can have R or S configuration, four diastereoisomeric 6-amino acids exist for any given side chain. Pro6 was first substituted with a range of cyclic 6-amino acids (FIG. 6). All these substitutions reduced the inhibitor activity at least 100-fold or inactivated it completely (90-94). The cyclic 6-amino acids apparently imposed conformational constraints onto FXII618 that hindered efficient binding to FXII. Peptides in which Pro6 was substituted with acyclic β-amino acids having methyl groups linked to either the carbon 3, carbon 2 or the amino group were subsequently synthesized (95-99; FIG. 6). It was reasoned that the methyl groups could replace interactions that were formed by one of the three carbon atoms in the side chain of proline. Three of these peptides had inhibitory constants in the medium micromolar range (95, 98, 99), but they all had weaker Kᵢs than the peptide with β-alanine in this position. The second one of the positions that were identified to tolerate the β-alanine substitution was Arg11. This amino acid was replaced with the β³-amino acid resembling structurally best L-Arg (S configuration in C3; (S)-β³-homoarginine) (100). This substitution yielded a peptide with a 4-fold improved (the bicyclic peptide is named FXII700; 5.4±0.1 nM; FIG. 6). Given that the two peptides with β-alanine and glycine in this position had a comparable affinity, it is likely that the large affinity improvement achieved with (S)-β³-homoarginine results from interactions of the arginine side chain that are better for the β³-amino acid than for L-Arg.

Another efficient synthetic strategy for inserting carbon atoms into the ring systems of bicyclic peptide FXII618 (1) is by substituting the cysteines with homocysteine or 5-mercapto-norvaline, having one and two additional carbons in the side chains, respectively. Six variants of FXII618 (1) were synthesized, each having one of the three cysteines replaced by either of the two cysteine homologues (FIG. 6). Replacement of the Cys12 with homocysteine improved the binding 1.3-fold, (Kᵢ of 105=16.3±3 nM). Replacement of the other two cysteines reduced the binding. The bicyclic peptide 105 having an excellent Kᵢ was named FXII701. The two modifications that gave excellent affinity improvement, Arg11 to (S)-β³-homoarginine and Cys12 to homocysteine, were subsequently combined. The resulting peptide (107) had a Kᵢ of 18±2 nM, showing that the two modifications provide no further affinity enhancement. Given the proximity of Arg11 and Cys12, it could be that the molecular basis for the affinity improvement achieved with the two modifications is related, and thus not additive. It is tempting to speculate that the insertion of a carbon atom in the two positions allows a small conformational change in this region of the macrocycle and that the quality of a molecular interaction, as for example an interaction of the arginine side chain, is improved.

In summary, the binding affinity of in vitro evolved cyclic peptide ligands can be improved by varying the size of the macrocyclic rings by one or two carbon atoms. It is reasoned that this chemical space is not sampled by screening genetically encoded cyclic peptide libraries and could potentially offer a rich source for slightly improved ligands. Indeed, synthesis and screening of only 34 peptide variants of the bicyclic peptide FXII inhibitor FXII618 yielded two inhibitors with substantially improved Kᵢs.

### EXAMPLE 4—SUBSTITUTION OF THE CONNECTING MOLECULE OF FXII618

Peptide FXII618 (RCFRLPCRQLRCR) was modified by the following linkers

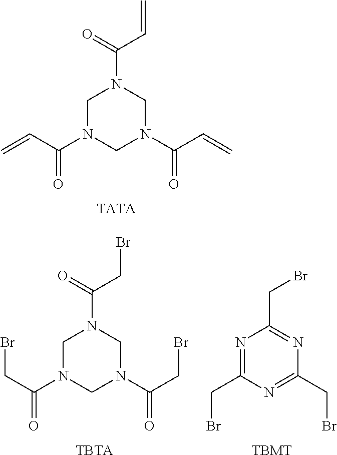

Modification Protocol:

The synthetic peptides were dissolved in aqueous buffer NH₄HCO₃ (60 mM) pH 8.0 at a final concentration of 1 mM. The linkers dissolved in acetonitrile were added to the peptides to obtain the final concentrations of 1.5 mM and 20% acetonitrile. The reaction solutions were kept in a 30° C. water bath for 1 h. The reaction product was purified by reversed-phase HPLC.

Protease Inhibition Assays:

Residual activities were measured in buffer (150 μL) containing 10 mM TrisCl, pH 7.4, 150 mM NaCl, 10 mM MgCl₂, 1 mM CaCl₂, 0.1% w/v bovine serum albumin (BSA), 0.01% v/v Triton-X100, and 5% v/v DMSO. Final concentrations of human factor XII beta (Innovative Research) 10 nM; Z-Gly-Gly-Arg-AMC (7-amino-4-methylcoumarin)-derived fluorogenic substrate (Bachem) were used at final concentrations of 50 μM. Fluorescence intensity was measured with a Tecan Infinite M 200 Pro plate reader (excitation at 368 nm, emission at 468 nm). The readings were measured in triplicate.

The inhibitory constant (Ki) was calculated according to the Cheng-Prusoff equation: IC₅₀/(1+([S]₀/Kₘ)), where IC₅₀ is the functional strength of the inhibitor, [S]₀ is the total substrate concentration, and K_{M} is the Michaelis-Menten constant. K_{M} for Z-Gly-Gly-Arg-AMC is 180 μM.

Kᵢ values:

FXII618-TATA: 24.9±5.7 nM

FXII618-TBAT: 338±32 nM

FXII618-TBMT: 1218±94 nM

The activity and specificity of FXII618-TATA, FXII618-TEAT and FXII618-TBMT are also shown in FIG. 14.

### EXAMPLE 5—VARIATION OF ARGININES IN FXII618 AND COMBINATION OF ADVANTAGEOUS AMINO ACID SUBSTITUTIONS IN FXII618

### Example 5.1—Variation of Amino Acids in Positions Arg8 and Arg11 of FXII618

These two positions were not exhaustively modified in the first affinity maturation efforts. Peptides with substitutions in these two positions were synthesized and tested for inhibition of human FXIIa, mouse FXIIa and for stability. The following peptide showed improved activity on mouse FXIIa and had a much improved stability.

| | | | Human | Mouse |
|---|---|---|---|---|
| | altered | amino acid | FXIIa Kᵢ | FXIIa Kᵢ |
| peptids | amino acid | substitution | [nM] | [nM] |
| JS17m | Arg8 | His | 21.6 ± 4 | 80 ± 30 |

### Example 5.2—Variation of Amino Acid Arg1 of FXII618 to Improve the Proteolytic Stability

To further increase the proteolytic stability of the inhibitor, additional amino acids at the N-terminus were tested. Specifically, D-amino acids were screened, wherein the L-Arg in position 1 (Arg1) was replaced by two D-amino acids. Among the Arg1 substitutions, the following peptide showed the best inhibitory activity.

| | | | Human |
|---|---|---|---|
| | altered | amino acid | FXIIa Kᵢ |
| peptide | amino acid | substitution | [nM] |
| JS7m | Arg1 | D-Arg-D-Ser | 18 ± 4 |

### Example 5.3—Combination of Beneficial Mutations

Overview of Amino Acid Substitutions in FXII618 (Living Substantial Improvements:

Arg1→D-Arg-D-Ser (rs) Kᵢ (human FXIIa)=18±4 nM

Phe3→4-fluoro-phenylalanine (F^{4F}) Kᵢ (human FXIIa)=2.0±0.3 nM

Arg8→His (H) Kᵢ (human FXIIa)=21.6±4 nM

Arg11→(S)-β3-homoarginine (R^{b}) Kᵢ (human FXIIa)=5.4±0.1 nM

Combination of 3 or 4 of the Beneficial Mutations:

| | | Human | t_{1/2} human |
|---|---|---|---|
| | Sequence | FXIIa Kᵢ [nM] | plasma (h) |
| FXII618 | RCFRLPCRQLRCR | 22 ± 4 | 3.9 ± 1.9 |
| JS33 | rsCF^{4F}RLPCHQLR^{b}CR | 0.36 ± 0.2 | 144 ± 30 |
| JS34 | rsCF^{4F}RLPCHQLRCR | 0.64 ± 0.1 | 38.7 |
| JS35 | rsCF^{4F}RLPCRQLR^{b}CR | 0.32 ± 0.1 | 21.9 |
| JS36 | rsCFRLPCHQLR^{b}CR | 1.0 ± 0.2 | N.D |
| JS33-R | rsCF^{4F}RLPCHQLR^{b}C | 0.4 ± 0.1 | 127 |

N.D. = not determined

### REFERENCES

(1) Behnke, C. A., Yee, V. C., Trong, I. L., Pedersen, L. C., Stenkamp, R. E., Kim, S. S., Reeck, G. R., and Teller, D. C. (1998) Structural determinants of the bifunctional corn Hageman factor inhibitor: x-ray crystal structure at 1.95 A resolution, Biochemistry 37, 15277-15288.
(2) Mahoney, W. C., Hermodson, M. A., Jones, B., Powers, D. D., Coffman, R. S., and Reeck, G. R. (1984) Amino acid sequence and secondary structural analysis of the corn inhibitor of trypsin and activated Hageman Factor, J Biol Chem 259, 8412-8416.
(3) Hansson, K. M., Nielsen, S., Elg, M., and Deinum, J. (2014) The effect of corn trypsin inhibitor and inhibiting antibodies for FXIa and FXIIa on coagulation of plasma and whole blood, Journal of thrombosis and haemostasis: JTH 12, 1678-1686.
(4) Hagedorn, I., Schmidbauer, S., Pleines, I., Kleinschnitz, C., Kronthaler, U., Stoll, G., Dickneite, G., and Nieswandt, B. (2010) Factor XIIa inhibitor recombinant human albumin Infestin-4 abolishes occlusive arterial thrombus formation without affecting bleeding, Circulation 121, 1510-1517.
(5) Kleinschnitz, C., Stoll, G., Bendszus, M., Schuh, K., Pauer, H. U., Burfeind, P., Renne, C., Gailani, D., Nieswandt, B., and Renne, T. (2006) Targeting coagulation factor XII provides protection from pathological thrombosis in cerebral ischemia without interfering with hemostasis. The Journal of experimental medicine 203, 513-518.
(6) Larsson, M., Rayzman, V., Nolte, M. W., Nickel, K. F., Bjorkqvist, J., Jamsa, A., Hardy, M. P., Fries, M., Schmidbauer, S., Hedenqvist, P., Broome, M., Pragst, I., Dickneite, G., Wilson, M. J., Nash, A. D., Panousis, C., and Renne, T. (2014) A factor XIIa inhibitory antibody provides thromboprotection in extracorporeal circulation without increasing bleeding risk, Science translational medicine 6, 222ra217.
(7) Matafonov, A., Leuno, P. Y., Gailani, A. E., Grach, S. L., Puy, C., Cheng, Q., Sun, M. F., McCarty, O. J., Tucker, E. I., Kataoka, H., Renne, T., Morrissey, J. H., Gruber, A., and Gailani, D. (2014) Factor XII inhibition reduces thrombus formation in a primate thrombosis model, Blood 123, 1739-1746.
(8) Renne, T., Pozgajova, M., Gruner, S., Schuh, K., Pauer, H. U., Burfeind, P., Gailani, D., and Nieswandt, B. (2005) Defective thrombus formation in mice lacking coagulation factor XII, The Journal of experimental medicine 202, 271-281.
(9) Woodruff, R. S., Xu, Y., Layzer, J., Wu, W., Ogletree, M. L., and Sullenger, B, A. (2013) Inhibiting the intrinsic pathway of coagulation with a factor XII-targeting RNA aptamer, Journal of thrombosis and haemostasis: JTH 11, 1364-1373.
(10) Xu, Y., Cai, T. O., Castriota, G., Zhou, Y., Hoos, L., Jochnowitz, N., Loewrigkeit, C., Cook, J. A., Wickham, A., Metzger, J. M., Ogletree, M. L., Seifert, D. A., and Chen, Z. (2014) Factor XIIa inhibition by Infestin-4: in vitro mode of action and in vivo antithrombotic benefit, Thrombosis and haemostasis 111, 694-704.
(11) Renne, T., Schmaier, A. H., Nickel, K. F., Blomback, M., and Maas, C. (2012) In vivo roles of factor XII, Blood 120, 4296-4303.
(12) Endler, G., Marsik, C., Jilma, B., Schickbauer, T., Quehenberger, P., and Mannhalter, C. (2007) Evidence of a U-shaped association between factor XII activity and overall survival, Journal of thrombosis and haemostasis: JTH 5, 1143-1148.
(13) Schloesser, M., Zeededer, S., Lutze, G., Halbmayer, W. M., Hofferbert, S., Hinney, B., Koestering, H., Lammie, B., Pindur, G., Thies, K., Kohler, M., and Engel, W. (1997) Mutations in the human factor XII gene, Blood 90, 3967-3977.
(14) Halbmayer, W. M., Haushofer, A., Schon, R., Mannhalter, C., Strohmer, E., Baumgarten, K., and Fischer, M. (1994) The prevalence of moderate and severe FXII (Hageman factor) deficiency among the normal population: evaluation of the incidence of FXII deficiency among 300 healthy blood donors, Thrombosis and haemostasis 71, 68-72.
(15) Campos, I, T., Amino, R., Sampaio, C. A., Auerswald, E. A., Friedrich, T., Lemaire, H. G., Schenkman, S., and Tanaka, A. S. (2002) Infestin, a thrombin inhibitor presents in Triatoma infestans midgut, a Chagas' disease vector: gene cloning, expression and characterization of the inhibitor. Insect Biochem Mol Biol 32, 991-997.
(16) Campos, I. T., Tanaka-Azevedo, A. M., and Tanaka, A. S (2004) Identification and characterization of a novel factor XIIa inhibitor in the hematophagous insect, Triatoma infestans (Hemiptera: Reduviidae), FEBS letters 577, 512-516. (17) Robert, S., Bertolla, C., Masereel, B., Dogne, J. M., and Pochet, L. (2008) Novel 3-carboxamide-coumarins as potent and selective FXIIa inhibitors, Journal of medicinal chemistry 51, 3077-3080.
(18) Baeriswyl, V., Calzavarini, S., Gerschheimer, C., Diderich, P., Angelillo-Scherrer, A., and Heinis, C, (2013) Development of a selective peptide macrocycle inhibitor of coagulation factor XII toward the generation of a safe antithrombotic therapy, Journal of medicinal chemistry 56, 3742-3746.
(19) Chen, S., Bertoldo, D., Angelini, A., Pojer, F., and Heinis, C. (2014) Peptide ligands stabilized by small molecules, Angewandte Chemie 53, 1602-1606.
(20) Chen, S., Morales-Sanfrutos, J., Angelini, A., Cutting. B., and Heinis, C, (2012) Structurally diverse cyclisation linkers impose different backbone conformations in bicyclic peptides, Chembiochem 13, 1032-1038.
(21) Hojima, Y., Pierce, J. V., and Pisano, J. J. (1980) Hageman factor fragment inhibitor in corn seeds: purification and characterization. Thrombosis research 20, 149-162,
(22) Spronk, H. M., Dielis, A. W., Panova-Noeva, M., van Oerle, R., Govers-Riemslag, J. W., Hamulyak, K., Falange, A., and Cate, H. T. (2009) Monitoring thrombin generation: is addition of corn trypsin inhibitor needed?, Thrombosis and haemostasis 101, 1156-1162.
(23) Laskowski, M., and Qasim, M. A. (2000) What can the structures of enzyme-inhibitor complexes tell us about the structures of enzyme substrate complexes?, Biochimica et biophysics acta 1477, 324-337.
(24) Van Veen, J. J., Gatt, A., Cooper, P. C., Kitchen, S., Bowyer, A. E., and Makris, M. (2008) Corn trypsin inhibitor in fluorogenic thrombin-generation measurements is only necessary at low tissue factor concentrations and influences the relationship between factor VIII coagulant activity and thromblogram parameters, Blood Coagul Fibrin 19, 183-189,
(25) Campo, G., Pavasini, R., Pollina, A., Fileti, L., Marchesini, J., Tebaldi, M., and Ferrari, R. (2012) Thrombin generation assay: a new tool to predict and optimize clinical outcome in cardiovascular patients?, Blood coagulation & fibrinolysis: an international journal in haemostasis and thrombosis 23, 680-687.
(26) Chitlur, M., Rivard, G. E., Lillicrap, D., Mann, K., Shima, M., Young, G., and Haemostasi, I. S. T. (2014) Recommendations for performing thromboelastography/thromboelastometry in hemophilia: communication from the SSC of the ISTH, Journal of Thrombosis and Haemostasis 12, 103-106.
(27) Alibeik, S., Zhu, S., Yau, J. W., Weitz, J. I., and Brash. J. L. (2011) Surface modification with polyethylene glycol-corn trypsin inhibitor conjugate to inhibit the contact factor pathway on blood-contacting surfaces, Acta biomaterialia 7, 4177-4186.
(28) Yau, J. W., Stafford, A. R., Liao, P., Fredenburgh, J. C., Roberts, R., Brash, J. L., and Weitz, J. I. (2012) Corn trypsin inhibitor coating attenuates the prothrombotic properties of catheters in vitro and in vivo, Acta biomaterialia 8, 4092-4100.
(29) Rentero Rebollo, I., and Heinis, C. (2013) Phage selection of bicyclic peptides, Methods 60, 46-54.
(30) Hemker, H. C., Giesen, P., Al Dieri, R., Regnault, V., de Smedt, E., Wagenvoord, R., Lecompte, T., and Beguin, S. (2003) Calibrated automated thrombin generation measurement in clotting plasma, Pathophysiology of haemostasis and thrombosis 33, 4-15.
(31) Seebach, ID.; Matthews, J. L. beta-peptides: a surprise at every turn. Chemical Communications 1997, 2015-2022.
(32) Horne, W. S.; Johnson, L. M.; Ketas, T. J.; Nasse, P. J.; Lu, M.; Moore, J. P.; Gellman, S, H. Structural and biological mimicry of protein surface recognition by alpha/beta-peptide foidamers. Proceedings of the National Academy of Sciences of the U.S. Pat. No. 2,009,106, 14751-14756.
(33) Johnson, L. M.; Gellman, S. H. alpha-Helix Mimicry with alpha/beta-Peptides. Methods in Protein Design 2013, 523, 407-429.
(34) Johnson, L. M.; Barrick, S.; Hager, M. V.; McFedries, A.; Homan, E. A.; Rabaglia, M. E.; Keller, M. P.; Attie, A, D.; Saghatelian, A.; Bisello, A.; Gellman, S. H. A Potent alphaibeta-Peptide Analogue of GLP-1 with Prolonged Action in Vivo. Journal of the American Chemical Society 2014, 136, 12848-12851.
(35) Cheloha, R. W.; Maeda, A.; Dean, T.; Gardella, T. J.; Gellman, S. H. Backbone modification of a polypeptide drug alters duration of action in vivo. Nature Biotechnology 2014, 32, 653-+.
(36) Checco, J. W.; Kreitler, D. F.; Thomas, N. C.; Belair, D. G.; Rettko, N. J.; Murphy, W. L.; Forest, K. T.; Gellman, S. H. Targeting diverse protein-protein interaction interfaces with alpha/beta-peptides derived from the Z-domain scaffold. Proceedings of the National Academy of Sciences of the U.S. Pat. No. 2,015,112, 4552-4557.
(37) Baeriswyl, V.; Calzavarini, S.; Chen, S.; Zorzi, A.; Bologna, L.; Angelillo-Scherrer, A.; Heinis, C. A Synthetic Factor XIIa Inhibitor Blocks Selectively Intrinsic Coagulation Initiation, ACS Chem Biol 2015, 10, 1861-70.

1. A bicyclic inhibitor of the coagulation enzyme activated factor XII (FXIIa) comprising or consisting of the peptide (X¹)(X²)(X³)ₙ(X⁴)RL(X⁵)(X⁶)ₘ(X⁷)(X⁹)ₗ(X¹⁰)(X¹¹)(X¹²)(X¹³)(X¹⁴)ₖ(X¹⁵)(X¹⁶), wherein
(X¹) is present or absent and, if present, is an amino acid;
(X²) is an amino acid with a side chain;
(X³) is an amino acid and n is between 0 and 3, preferably 0 or 1 and most preferably 0;
(X⁴) is an aliphatic L-amino acid or a cyclic L-amino acid, preferably L, P or an aromatic L-amino acid, and most preferably an aromatic L-amino acid;
(X⁵) is an amino acid;
(X⁶) is an amino acid and m is between 0 and 3, preferably 0 or 1 and most preferably 0;
(X⁷) is an amino acid with a side chain;
(X⁹) is an amino acid and I is between 0 and 3, preferably 0 or 1 and most preferably 0;
(X¹⁰) is an amino acid;
(X¹¹) is an amino acid, preferably Q;
(X¹²) is a hydrophobic L-amino acid, preferably an aliphatic L-amino acid, and is most preferably L;
(X¹³) is an amino acid;
(X¹⁴) is an amino acid and k is between 0 and 3, preferably 0 or 1 and most preferably 0, (X¹⁵) is an amino acid with a side chain; and
(X¹⁶)) is present or absent and, if present, is an amino acid; and
wherein the side chains of (X²), (X⁷) and (X¹⁵) are connected via a connecting molecule, said connecting molecule having at least three functional groups, each functional group forming a covalent bond with one of the side chains of (X²), (X⁷) and (X¹⁵).

2. The inhibitor of claim 1, wherein (X⁴) is selected from the group consisting of L, P, F, W, Y, 1-naphthylalanine, 2-naphthylalanine, 3-benzothienylalanine, 3-fluoro-phenylalanine, 3-methyl-phenylalanine, 2-amino-3-(pyridine-3-yl)propionic acid, 2-fluoro-phenylalanine, 4-fluoro-phenylalanine, and 2-nitro-phenylalanine.

3. The inhibitor of claim 1, wherein (X⁵) is a hydrophobic L-amino acid or a polar, uncharged L-amino acid.

4. The inhibitor of claim 1, wherein the side chains of (X²), (X⁷) and (X¹⁵) comprise a functional group independently selected from —NH₂, —COOH, —SH, alkene, alkyne, azide and chloroacetamide.

5. The inhibitor of claim 1, wherein (X²), (X⁷) and (X¹⁵) are each independently K, ornithine, thialysine, 2,3-diaminopropanoic acid, diaminobutyric acid, D, E, C, homocysteine, penicillamine or propargylglycine.

6. The inhibitor of claim 1, wherein
(X²) is 5-mercapto-norvaline, homocysteine or C; and/or
(X⁷) is homocysteine or C; and/or
(X¹⁵) is 5-mercapto-norvaline, homocysteine or C.

7. The inhibitor of claim 1, wherein
(X¹) is D-Arg, homoarginine, L, norarginine, 4-guanidinophenylalanine, homolysine, D-Arg-D-Ser or L-Arg; and/or
(X¹⁰) is G, H, or R;
(X¹³) is A, G, (S)-β3-homoarginine or R; and/or
(X¹⁶) is R or absent.

8. The inhibitor of claim 1, wherein the connecting molecule is selected from 1,3,5-triacryloyl-1,3,5-triazinane (TATA), 1,3,5-tris(chloroacetyl)-1,3,5-triazinane (TCAT), 1,3,5-tris(bromoacetyl)-1,3,5-triazinane (TBAT), 1,3,5-tris(bromomethyl)benzene (TBMB) and 2,4,6-tris(bromomethyl)-1,3,5-triazine (TBMT).

9. The inhibitor of claim 1, wherein at least one of the following items (i) to (iv) applies:
(i) (X¹) is D-Arg-D-Ser,
(ii) (X⁴) is 4-fluoro-phenylalanine,
(iii) (X¹⁰) is H, and/or
(iv) (X¹³) is (S)-β3-homoarginine;
wherein (X¹⁶) is optionally absent, and the connecting molecule is TATA.

10. The inhibitor of claim 1, wherein the inhibitor has an inhibitory constant (Kᵢ) for FXIIa of less than 100 nM.

11. An ex vivo method of inhibiting the enzymatic activity of FXIIa comprising contacting the inhibitor of claim 1 with FXIIa, wherein FXIIa is present in a blood, plasma or serum sample.

12. A pharmaceutical composition comprising the inhibitor of claim 1.

13.-14. (canceled)

15. A kit for testing blood coagulation comprising the inhibitor of claim 1.
